(19) United States
(12) Patent Application Publication
(10) Pub. No.: US 2014/0206849 A1
(43) Pub. Date: Jul. 24, 2014

(54) ANTIBODIES HAVING REDUCED IMMUNOGENICITY IN A HUMAN

(71) Applicant: Paul P. Tamburini, Kensington, CT (US)

(72) Inventors: Russell P. Rother, Oklahoma City, OK (US); Paul P. Tamburini, Kensington, CT (US)

(73) Assignee: ALEXION PHARMACEUTICALS, INC., Cheshire, CT (US)

(21) Appl. No.: 13/695,250

(22) PCT Filed: Apr. 29, 2011
(86) PCT No.: PCT/US2011/034598

§ 371 Date: Aug. 21, 2013

Related U.S. Application Data
(60) Provisional application No. 61/330,261, filed on Apr. 30, 2010.

(51) Int. Cl. C07K 16/18

(52) U.S. Cl. CPC C07K 16/18
USPC 530/389.1; 536/23.53

(57) ABSTRACT

The disclosure relates to engineered antibodies that when administered to a human, exhibit a low level of immunogenicity in the human. The disclosure also relates to methods for generating the antibodies. The engineered antibodies can be derived from, e.g., on-human (e.g., murine) donor antibodies or from chimeric or humanized antibodies that, when chronically administered to a human, are known to, are predicted to, or are expected to, elicit a neutralizing anti-antibody response in the human.

### RELATED APPLICATIONS

This application claims the benefit of U.S. Provisional Application No. 61/330,261, filed on Apr. 30, 2010. All the teachings of the above-referenced application are incorporated herein by reference.

### SEQUENCE LISTING

The instant application contains a Sequence Listing which has been submitted via EFS-Web and is hereby incorporated by reference in its entirety. Said ASCII copy, created on Apr. 29, 2011, is named ALXN155W.txt, and is 29,908 bytes in size.

### TECHNICAL FIELD

The field of the invention is medicine, immunology, molecular biology, and protein chemistry.

### BACKGROUND

Administration of rodent antibodies (e.g., mouse, rat, or rabbit) to a human generally results in the production in the human of anti-rodent immunoglobulin antibodies. The anti-rodent antibodies can neutralize any potential therapeutic benefit of the therapeutic antibodies. The same process occurs for other types of non-human antibodies (e.g., simian antibodies) administered to a human. To overcome this problem, the non-human antibodies can be re-engineered as, e.g., chimeric human antibodies or CDR-grafted human antibodies. In a human chimeric antibody, the variable regions are of non-human origin (e.g., mouse origin) and the constant regions are of human origin. The generation of CDR-grafted antibodies, often referred to as humanized antibodies, is a more complex process, wherein the CDRs of a fully human acceptor antibody are replaced with the CDRs of a non-human donor antibody. However, human anti-human antibody (HAHA) responses are still reported for each of these re-engineered antibody variants. For example, Welt et al. [(2003) Clin Cancer Res 9:1338-1346] describes that a humanized anti-A33 antibody, when administered to human colon cancer patients, elicited a HAHA response in 73% of the patients. In another example, the chimeric anti-TNF antibody Remicade® (Johnson & Johnson) has been shown to provoke a HAHA response in up to 53% of rheumatoid arthritis patients on monotherapy and as high as 15% of patients when administered in combination therapy with methotrexate. (See, e.g., Aarden et al. (2008) Curr Opin Immunol 20:431-435.) As high as 26% of patients with ankylosing spondylitis were found to develop antibodies to Remicade® upon repeated administration of the drug. Anderson [(2005) Semin Arthritis Rheum 34:19-22] reported that in patients receiving adalimumab (HUMIRA®), a fully humanized antibody, the incidence of human anti-adalimumab antibodies is around 6%. As with Remicade®, a lower incidence of HAHA response against adalimumab was observed when the antibody was administered in combination with methotrexate (see Aarden et al. (2008), supra). However, Aarden et al. found that nearly 20% of the HAHA responses to adalimumab were neutralizing. Thus, there clearly remains a need for improved methods for humanizing therapeutic antibodies to reduce immunogenicity in human patients, particularly for therapeutic antibodies that are to be chronically administered.

### SUMMARY

The present disclosure is based, at least in part, on the discovery by the inventors that the humanized anti-C5 antibody eculizumab exhibits a very low level of immunogenicity in humans. As detailed in the accompanying working examples, over 130 therapeutic doses of eculizumab were administered to individual patients with paroxysmal nocturnal hemoglobinuria (PNH) over the course of several years. The patients were not concurrently administered an immunosuppressant such as methotrexate. Blood samples were obtained from the patients and analyzed to determine whether the samples contained antibodies that bound to eculizumab. The presence of such antibodies is indicative of a human anti-human antibody response against eculizumab. It was found that only 1.2% of the patients (2 of 161) had low, but detectable levels of antibodies that bound to eculizumab. However, further analysis confirmed that neither of the two blood samples contained antibodies that were capable of neutralizing the therapeutic efficacy of eculizumab. Thus, the inventors reasoned that eculizumab can be used as a scaffold to create additional, therapeutic antibodies that also exhibit a low level of immunogenicity in a human. Accordingly, the disclosure features engineered antibodies, which contain the CDRs of a donor antibody grafted onto a reduced immunogenicity acceptor antibody scaffold and are less immunogenic in a human as compared to the immunogenicity of the donor antibody in a human. The engineered antibodies can be derived from donor antibodies that are known, are predicted, or are expected, to elicit a neutralizing anti-antibody response in the human, especially when they are chronically administered. As described herein, the donor antibody can be, e.g., a non-human antibody (e.g., a rodent antibody or a non-human primate antibody) or a humanized or fully human antibody that is found to generate a human anti-human antibody (HAHA) response (e.g., a HAHA response that neutralizes the therapeutic efficacy of the donor antibody in a human). The donor antibody and/or the resulting engineered antibody can be an antibody that is useful for treating or diagnosing any of a variety of diseases in a human subject including, without limitation, a cancer, an infection, a metabolic disorder, an inflammatory condition, an autoimmune disease, a neurological disorder, a hematological disorder, and a cardiovascular disorder.

As discussed in the working Examples, eculizumab is a humanized antibody having a set of light chain framework regions derived from the I.23 Ig light chain molecule and a set of heavy chain framework regions derived from the H20C3 Ig heavy chain molecule. The amino acid sequence of the H20C3 heavy chain polypeptide is provided in Weng et al. (1992) J Immunol 149(7):2518-2529 and is also available under NCBI Accession No. AAA52985. The nucleic acid sequence encoding H20C3 is approximately 98% similar to counterpart human germline heavy chain immunoglobulin genes. The amino acid sequence for the I.23 light chain polypeptide is set forth partially in Klein et al. (1993) Eur J Immunol 23(12):3248-3262 and the complete sequence is also publicly available under NCBI Accession No. CAA51145.1. The I.23 coding sequence was derived from human germline V_{κ} and J_{κ} genes, and contains but a single amino acid change from the germline sequences at position 38. Accordingly, framework regions from eculizumab (the light chain or heavy chain variable region of eculizumab), I.23, and/or H20C3 can be used in the generation of engineered antibodies that exhibit a low level of immunogenicity in a human. The working examples describe the construction of an additional, functional humanized antibody that includes light chain framework regions 1 to 3 and heavy chain framework regions 1 to 3 from eculizumab. In some embodiments, one or more, but not all, of the CDRs of eculizumab, I.23, and/or H20C3 can also be used in the generation of the engineered antibodies.

In one aspect, the disclosure features a polypeptide (e.g., a light chain polypeptide) comprising the following amino acid sequence segments in order: LFR1-LCDR1-LFR2-LCDR2-LFR3-LCDR3-LFR4. One or more of light chain framework regions LFR1, LFR2, and LFR3 are obtained from a light chain variable region having the amino acid sequence depicted in SEQ ID NO:2 or SEQ ID NO:8, and one or more of the light chain complementarity determining regions LCDR1, LCDR2, and LCDR3 are obtained from a donor antibody, with the proviso that the polypeptide does not comprise the complete amino acid sequence depicted in SEQ ID NO:2 or SEQ ID NO:8. In some embodiments, LFR4 can be obtained from the light chain variable region having the amino acid sequence depicted in SEQ ID NO:2 or SEQ ID NO:8.

In some embodiments, one of the CDRs can be from the light chain variable region having the amino acid sequence depicted in SEQ ID NO:2 or SEQ ID NO:8. In some embodiments, two of the CDRs can be from the light chain variable region having the amino acid sequence depicted in SEQ ID NO:2 or SEQ ID NO:8. In some embodiments, at least two of the CDRs can be from the same donor antibody. In some embodiments, all of the CDRs can be from the same donor antibody.

In some embodiments, the framework regions and the CDRs are defined according to Kabat. In some embodiments, the framework regions and the CDRs are defined according to Chothia. In some embodiments, the framework regions and the CDRs are defined according to the combined Kabat-Chothia definition.

In some embodiments, LFR1 comprises, or consists of, the amino acid sequence depicted in SEQ ID NO:9. In some embodiments, LFR2 comprises, or consists of, the amino acid sequence depicted in SEQ ID NO:10 or SEQ ID NO:18. In some embodiments, LFR3 comprises, or consists of, the amino acid sequence depicted in SEQ ID NO:11. In some embodiments, LFR4 comprises, or consists of, the amino acid sequence depicted in SEQ ID NO:12.

In some embodiments, LFR1 comprises, or consists of, the amino acid sequence depicted in SEQ ID NO:9; LFR2 comprises, or consists of, the amino acid sequence depicted in SEQ ID NO:10; LFR3 comprises, or consists of, the amino acid sequence depicted in SEQ ID NO:11; and LFR4 comprises, or consists of, the amino acid sequence depicted in SEQ ID NO:12.

In some embodiments, LFR1 comprises, or consists of, the amino acid sequence depicted in SEQ ID NO:9; LFR2 comprises, or consists of, the amino acid sequence depicted in SEQ ID NO:18; LFR3 comprises, or consists of, the amino acid sequence depicted in SEQ ID NO:11; and LFR4 comprises, or consists of, the amino acid sequence depicted in SEQ ID NO:12.

In some embodiments, LFR1 comprises, or consists of, the amino acid sequence depicted in SEQ ID NO:20 or SEQ ID NO:24. In some embodiments, LFR2 comprises, or consists of, the amino acid sequence depicted in SEQ ID NO:21 or SEQ ID NO:25. In some embodiments, LFR3 comprises, or consists of, the amino acid sequence depicted in SEQ ID NO:22 or SEQ ID NO:26. In some embodiments, LFR4 comprises, or consists of, the amino acid sequence depicted in SEQ ID NO:23.

In some embodiments, LFR1 comprises, or consists of, the amino acid sequence depicted in SEQ ID NO:20; LFR2 comprises, or consists of, the amino acid sequence depicted in SEQ ID NO:21; LFR3 comprises, or consists of, the amino acid sequence depicted in SEQ ID NO:22; and LFR4 comprises, or consists of, the amino acid sequence depicted in SEQ ID NO:23.

In some embodiments, LFR1 comprises, or consists of, the amino acid sequence depicted in SEQ ID NO:24; LFR2 comprises, or consists of, the amino acid sequence depicted in SEQ ID NO:25; LFR3 comprises, or consists of, the amino acid sequence depicted in SEQ ID NO:26; and LFR4 comprises, or consists of, the amino acid sequence depicted in SEQ ID NO:23.

In some embodiments, the polypeptide (e.g., the light chain polypeptide) comprises all or part of an immunoglobulin light chain polypeptide constant region, e.g., the polypeptide can comprise the amino acid sequence depicted in SEQ ID NO:3. In some embodiments, the light chain polypeptide constant region comprises a human amino acid sequence. In some embodiments, the light chain constant region is a λ light chain constant region or a K light chain constant region.

In another aspect, the disclosure features a polypeptide (e.g., a heavy chain polypeptide) comprising, or consisting of, the following amino acid sequence segments in order: HFR1-HCDR1-HFR2-HCDR2-HFR3-HCDR3-HFR4. One or more of heavy chain framework regions HFR1, HFR2, and HFR3 can be, or are, obtained from a heavy chain variable region having the amino acid sequence depicted in SEQ ID NO:5 or SEQ ID NO:7, and one or more of the heavy chain complementarity determining regions HCDR1, HCDR2, and HCDR3 are obtained from a donor antibody, with the proviso that the polypeptide does not comprise the complete amino acid sequence depicted in SEQ ID NO:5 or SEQ ID NO:7. In some embodiments, LFR4 can be obtained from the heavy chain variable region having the amino acid sequence depicted in SEQ ID NO:5 or SEQ ID NO:7.

In some embodiments, one of the CDRs can be from the heavy chain variable region having the amino acid sequence depicted in SEQ ID NO:5 or SEQ ID NO:7. In some embodiments, two of the CDRs can be from the heavy chain variable region having the amino acid sequence depicted in SEQ ID NO:5 or SEQ ID NO:7. In some embodiments, at least two of the CDRs can be from the same donor antibody. In some embodiments, all of the CDRs can be from the same donor antibody.

In some embodiments, the framework regions and the CDRs are defined according to Kabat. In some embodiments, the framework regions and the CDRs are defined according to Chothia. In some embodiments, the framework regions and the CDRs are defined according to the combined Kabat-Chothia definition.

In some embodiments, HFR1 comprises, or consists of, the amino acid sequence depicted in any one of SEQ ID NOs: 13, 17, or 19. In some embodiments, HFR2 comprises, or consists of, the amino acid sequence depicted in SEQ ID NO:14. In some embodiments, HFR3 comprises, or consists of, the amino acid sequence depicted in SEQ ID NO:15. In some embodiments, HFR4 comprises, or consists of, the amino acid sequence depicted in SEQ ID NO:16.

In some embodiments, HFR1 comprises, or consists of, the amino acid sequence depicted in SEQ ID NO:13; HFR2 comprises, or consists of, the amino acid sequence depicted in SEQ ID NO:14; HFR3 comprises, or consists of, the amino acid sequence depicted in SEQ ID NO:15; and HFR4 comprises, or consists of, the amino acid sequence depicted in SEQ ID NO:16.

In some embodiments, HFR1 comprises, or consists of, the amino acid sequence depicted in SEQ ID NO:17; HFR2 comprises, or consists of, the amino acid sequence depicted in SEQ ID NO:14; HFR3 comprises, or consists of, the amino acid sequence depicted in SEQ ID NO:15; and HFR4 comprises, or consists of, the amino acid sequence depicted in SEQ ID NO:16.

In some embodiments, HFR1 comprises, or consists of, the amino acid sequence depicted in SEQ ID NO:19; HFR2 comprises, or consists of, the amino acid sequence depicted in SEQ ID NO:14; HFR3 comprises, or consists of, the amino acid sequence depicted in SEQ ID NO:15; and HFR4 comprises, or consists of, the amino acid sequence depicted in SEQ ID NO:16.

In some embodiments, HFR1 comprises, or consists of, the amino acid sequence depicted in SEQ ID NO:17. In some embodiments, HFR2 comprises, or consists of, the amino acid sequence depicted in SEQ ID NO:27 or SEQ ID NO:30. In some embodiments, HFR3 comprises, or consists of, the amino acid sequence depicted in SEQ ID NO:28 or SEQ ID NO:31. In some embodiments, HFR4 comprises, or consists of, the amino acid sequence depicted in SEQ ID NO:29 or SEQ ID NO:32.

In some embodiments, HFR1 comprises, or consists of, the amino acid sequence depicted in SEQ ID NO:17; HFR2 comprises, or consists of, the amino acid sequence depicted in SEQ ID NO:27; HFR3 comprises, or consists of, the amino acid sequence depicted in SEQ ID NO:28; and HFR4 comprises, or consists of, the amino acid sequence depicted in SEQ ID NO:29.

In some embodiments, HFR1 comprises, or consists of, the amino acid sequence depicted in SEQ ID NO:17; HFR2 comprises, or consists of, the amino acid sequence depicted in SEQ ID NO:30; HFR3 comprises, or consists of, the amino acid sequence depicted in SEQ ID NO:31; and HFR4 comprises, or consists of, the amino acid sequence depicted in SEQ ID NO:32.

In some embodiments, the polypeptide (e.g., the heavy chain polypeptide) comprises all or part of an immunoglobulin heavy chain polypeptide constant region, e.g., the polypeptide can comprise the amino acid sequence depicted in SEQ ID NO:6. In some embodiments, the polypeptide (e.g., the heavy chain polypeptide) can comprise an Fc portion of an immunoglobulin molecule. In some embodiments, the immunoglobulin heavy chain polypeptide constant region is an IgG, IgA, IgE, IgD, or IgM heavy chain polypeptide constant region.

In another aspect, the disclosure features an engineered antibody comprising: (i) a light chain polypeptide and (ii) a heavy chain polypeptide, wherein the light chain polypeptide comprises the following amino acid sequence segments in order: LFR1-LCDR1-LFR2-LCDR2-LFR3-LCDR3-LFR4, wherein light chain framework regions LFR1, LFR2, and LFR3 are obtained from a light chain variable region having the amino acid sequence depicted in SEQ ID NO:2 or SEQ ID NO:8, and wherein one or more of the light chain complementarity determining regions LCDR1, LCDR2, and LCDR3 are obtained from a donor antibody, with the proviso that the light chain polypeptide does not comprise the complete amino acid sequence depicted in SEQ ID NO:2 or SEQ ID NO:8. The heavy chain polypeptide comprises the following amino acid sequence segments in order: HFR1-HCDR1-HFR2-HCDR2-HFR3-HCDR3-HFR4, wherein heavy chain framework regions HFR1, HFR2, and HFR3 are obtained from a heavy chain variable region having the amino acid sequence depicted in SEQ ID NO:5 or SEQ ID NO:7, and wherein one or more of the heavy chain complementarity determining regions HCDR1, HCDR2, and HCDR3 are obtained from a donor antibody, with the proviso that the heavy chain polypeptide does not comprise the complete amino acid sequence depicted in SEQ ID NO:5 or SEQ ID NO:7.

In some embodiments, the light chain framework regions, the heavy chain framework regions, the light chain CDRs, and the heavy chain CDRs are defined according to the Kabat definition. In some embodiments, the light chain framework regions, the heavy chain framework regions, the light chain CDRs, and the heavy chain CDRs are defined according to the Chothia definition. In some embodiments, the light chain framework regions, the heavy chain framework regions, the light chain CDRs, and the heavy chain CDRs are defined according to a combined Kabat-Chothia definition.

In some embodiments, LFR1 comprises the amino acid sequence depicted in SEQ ID NO:9; LFR2 comprises the amino acid sequence depicted in SEQ ID NO:10; LFR3 comprises the amino acid sequence depicted in SEQ ID NO:11; LFR4 comprises the amino acid sequence depicted in SEQ ID NO:12, HFR1 comprises the amino acid sequence depicted in SEQ ID NO:13; HFR2 comprises the amino acid sequence depicted in SEQ ID NO:14; HFR3 comprises the amino acid sequence depicted in SEQ ID NO:15; and HFR4 comprises the amino acid sequence depicted in SEQ ID NO:16.

In some embodiments, LFR1 comprises the amino acid sequence depicted in SEQ ID NO:9; LFR2 comprises the amino acid sequence depicted in SEQ ID NO:18; LFR3 comprises the amino acid sequence depicted in SEQ ID NO:11; LFR4 comprises the amino acid sequence depicted in SEQ ID NO:12, HFR1 comprises the amino acid sequence depicted in SEQ ID NO:19; HFR2 comprises the amino acid sequence depicted in SEQ ID NO:14; HFR3 comprises the amino acid sequence depicted in SEQ ID NO:15; and HFR4 comprises the amino acid sequence depicted in SEQ ID NO:16.

In some embodiments, LFR1 comprises the amino acid sequence depicted in SEQ ID NO:9; LFR2 comprises the amino acid sequence depicted in SEQ ID NO:18; LFR3 comprises the amino acid sequence depicted in SEQ ID NO:11; LFR4 comprises the amino acid sequence depicted in SEQ ID NO:12, HFR1 comprises the amino acid sequence depicted in SEQ ID NO:13; HFR2 comprises the amino acid sequence depicted in SEQ ID NO:14; HFR3 comprises the amino acid sequence depicted in SEQ ID NO:15; and HFR4 comprises the amino acid sequence depicted in SEQ ID NO:16.

In some embodiments, LFR1 comprises the amino acid sequence depicted in SEQ ID NO:9; LFR2 comprises the amino acid sequence depicted in SEQ ID NO:10; LFR3 comprises the amino acid sequence depicted in SEQ ID NO:11; LFR4 comprises the amino acid sequence depicted in SEQ ID NO:12, HFR1 comprises the amino acid sequence depicted in SEQ ID NO:19; HFR2 comprises the amino acid sequence depicted in SEQ ID NO:14; HFR3 comprises the amino acid sequence depicted in SEQ ID NO:15; and HFR4 comprises the amino acid sequence depicted in SEQ ID NO:16.

In some embodiments, LFR1 comprises the amino acid sequence depicted in SEQ ID NO:9; LFR2 comprises the amino acid sequence depicted in SEQ ID NO:10; LFR3 comprises the amino acid sequence depicted in SEQ ID NO:11; LFR4 comprises the amino acid sequence depicted in SEQ ID NO:12, HFR1 comprises the amino acid sequence depicted in SEQ ID NO:17; HFR2 comprises the amino acid sequence depicted in SEQ ID NO:14; HFR3 comprises the amino acid sequence depicted in SEQ ID NO:15; and HFR4 comprises the amino acid sequence depicted in SEQ ID NO:16.

In some embodiments, LFR1 comprises the amino acid sequence depicted in SEQ ID NO:9; LFR2 comprises the amino acid sequence depicted in SEQ ID NO:18; LFR3 comprises the amino acid sequence depicted in SEQ ID NO:11; LFR4 comprising the amino acid sequence depicted in SEQ ID NO:12, HFR1 comprises the amino acid sequence depicted in SEQ ID NO:17; HFR2 comprises the amino acid sequence depicted in SEQ ID NO:14; HFR3 comprises the amino acid sequence depicted in SEQ ID NO:15; and HFR4 comprises the amino acid sequence depicted in SEQ ID NO:16.

In some embodiments, LFR1 comprises the amino acid sequence depicted in SEQ ID NO:20; LFR2 comprises the amino acid sequence depicted in SEQ ID NO:21; LFR3 comprises the amino acid sequence depicted in SEQ ID NO:22; LFR4 comprising the amino acid sequence depicted in SEQ ID NO:23, HFR1 comprises the amino acid sequence depicted in SEQ ID NO:17; HFR2 comprises the amino acid sequence depicted in SEQ ID NO:27; HFR3 comprises the amino acid sequence depicted in SEQ ID NO:28; and HFR4 comprises the amino acid sequence depicted in SEQ ID NO:29.

In some embodiments, LFR1 comprises the amino acid sequence depicted in SEQ ID NO:20; LFR2 comprises the amino acid sequence depicted in SEQ ID NO:21; LFR3 comprises the amino acid sequence depicted in SEQ ID NO:22; LFR4 comprising the amino acid sequence depicted in SEQ ID NO:23, HFR1 comprises the amino acid sequence depicted in SEQ ID NO:17; HFR2 comprises the amino acid sequence depicted in SEQ ID NO:30; HFR3 comprises the amino acid sequence depicted in SEQ ID NO:31; and HFR4 comprises the amino acid sequence depicted in SEQ ID NO:32.

In some embodiments, LFR1 comprises the amino acid sequence depicted in SEQ ID NO:24; LFR2 comprises the amino acid sequence depicted in SEQ ID NO:25; LFR3 comprises the amino acid sequence depicted in SEQ ID NO:26; LFR4 comprising the amino acid sequence depicted in SEQ ID NO:23, HFR1 comprises the amino acid sequence depicted in SEQ ID NO:17; HFR2 comprises the amino acid sequence depicted in SEQ ID NO:27; HFR3 comprises the amino acid sequence depicted in SEQ ID NO:28; and HFR4 comprises the amino acid sequence depicted in SEQ ID NO:29.

In some embodiments, LFR1 comprises the amino acid sequence depicted in SEQ ID NO:24; LFR2 comprises the amino acid sequence depicted in SEQ ID NO:25; LFR3 comprises the amino acid sequence depicted in SEQ ID NO:26; LFR4 comprising the amino acid sequence depicted in SEQ ID NO:23, HFR1 comprises the amino acid sequence depicted in SEQ ID NO:17; HFR2 comprises the amino acid sequence depicted in SEQ ID NO:30; HFR3 comprises the amino acid sequence depicted in SEQ ID NO:31; and HFR4 comprises the amino acid sequence depicted in SEQ ID NO:32.

In some embodiments, the engineered antibody comprises a paired set of heavy chain framework regions and light chain framework regions depicted in Table 5.

In some embodiments, the engineered antibody can be, e.g., an antibody fragment, e.g., an antibody fragment selected from the group consisting of an Fd fragment, an Fab fragment, an Fab′ fragment, and an F(ab′)₂ fragment.

In some embodiments of any of the engineered antibodies described herein, the light chain polypeptide and the heavy chain polypeptide can be covalently bound to each other.

In some embodiments, the engineered antibody binds to a complement component protein. The complement component protein can be one selected from the group consisting of C1q, C1r, C1s, C4, C4a, C4b, C3, C3a, C3b, C2, C2a, C2b, C5, C5a, C5b, C6, C7, C8, C9, properdin, complement factor D, complement factor B, MBL, MASP1, MASP2, and MASP3.

In some embodiments, the engineered antibody binds to a cell surface receptor, e.g., a G protein coupled receptor, a chemokine receptor, a cytokine receptor, or a receptor tyrosine kinase.

In some embodiments, the engineered antibody binds to: (i) a death receptor or (ii) a ligand of a death receptor. In some embodiments, the engineered antibody binds to a growth factor, a chemokine, or a cytokine. In some embodiments, the engineered antibody binds to an immunoglobulin molecule, e.g., an IgE molecule.

In yet another aspect, the disclosure features a nucleic acid encoding: (i) any one of the polypeptides described herein (e.g., a light chain polypeptide or a heavy chain polypeptide) or (ii) any of the engineered antibodies described herein. Also featured is a vector comprising the nucleic acid. The vector can be an expression vector. In addition, the disclosure features a cell comprising the nucleic acid or the vector. In another aspect, the disclosure features a method for producing a polypeptide or an engineered antibody. The method includes culturing the aforementioned cell containing the vector under conditions suitable to allow for expression by the cell of the polypeptide or the engineered antibody encoded by the nucleic acid contained within the vector. The method can also include isolating the polypeptide or engineered antibody from the cultured cells or from the medium in which the cells were cultured. Also featured is an isolated polypeptide or an isolated engineered antibody produced by the foregoing method.

In another aspect, the disclosure features a method for generating an engineered light chain antibody variable region that is less immunogenic in a human as compared to the immunogenicity of a donor light chain variable region. The method includes: providing information comprising: (i) an acceptor light chain antibody variable region amino acid sequence comprising the amino acid sequence depicted in SEQ ID NO:2 or SEQ ID NO:8 or (ii) a nucleic acid sequence encoding the acceptor light chain antibody variable region amino acid sequence; providing information comprising: (iii) at least one donor antibody light chain variable region amino acid sequence or (iv) a nucleic acid sequence encoding the donor antibody light chain variable region amino acid sequence; replacing one or more CDRs of the acceptor light chain antibody variable region with one or more CDRs from the donor antibody light chain variable region to thereby generate an engineered light chain variable region that is less immunogenic in a human as compared to the immunogenicity of the donor antibody light chain variable region, with the proviso that the engineered light chain variable region does not comprise a light chain polypeptide comprising the complete amino acid sequence depicted in SEQ ID NO:2 or SEQ ID NO:8. The method can include obtaining a heavy chain antibody variable region, or a nucleic acid encoding a heavy chain antibody variable region, that is complementary to the engineered light chain antibody variable region to thereby generate an engineered antibody.

In some embodiments of the foregoing methods, guided selection is used to obtain the heavy chain antibody variable region.

In some embodiments of the foregoing methods, the heavy chain antibody variable region is an engineered heavy chain antibody variable region.

In some embodiments of the foregoing methods, the generation of the engineered heavy chain antibody variable region includes: providing information comprising: (i) an acceptor heavy chain antibody variable region amino acid sequence comprising the amino acid sequence depicted in SEQ ID NO:5 or SEQ ID NO:7 or (ii) a nucleic acid sequence encoding the acceptor heavy chain antibody variable region amino acid sequence; providing information comprising: (iii) at least one donor antibody heavy chain variable region amino acid sequence or (iv) a nucleic acid sequence encoding the donor antibody heavy chain variable region amino acid sequence; replacing one or more CDRs of the acceptor heavy chain antibody variable region with one or more CDRs from the donor antibody heavy chain variable region to thereby generate an engineered heavy chain antibody variable region that is less immunogenic in a human as compared to the immunogenicity of the donor antibody heavy chain variable region, with the proviso that the engineered antibody variable region does not comprise a heavy chain polypeptide variable region comprising the complete amino acid sequence depicted in SEQ ID NO:5 or SEQ ID NO:7.

In yet another aspect, the disclosure features a method for generating an engineered heavy chain antibody variable region that is less immunogenic in a human as compared to the immunogenicity of a donor antibody heavy chain variable region. The method includes: providing information comprising: (i) an acceptor heavy chain antibody variable region amino acid sequence comprising the amino acid sequence depicted in SEQ ID NO:5 or SEQ ID NO:7 or (ii) a nucleic acid sequence encoding the acceptor heavy chain antibody variable region amino acid sequence; providing information comprising: (iii) at least one donor antibody heavy chain variable region amino acid sequence or (iv) a nucleic acid sequence encoding the donor antibody heavy chain variable region amino acid sequence; replacing one or more CDRs of the acceptor heavy chain antibody variable region with one or more CDRs from the donor antibody heavy chain variable region to thereby generate an engineered heavy chain antibody variable region that is less immunogenic in a human as compared to the immunogenicity of the donor antibody heavy chain variable region, with the proviso that the engineered antibody variable region does not comprise a heavy chain polypeptide variable region comprising the complete amino acid sequence depicted in SEQ ID NO:5 or SEQ ID NO:7. The method can include obtaining a light chain antibody variable region complementary to the engineered heavy chain antibody variable region to thereby generate an engineered antibody.

In some embodiments of the foregoing methods, guided selection is used to obtain the engineered light chain antibody variable region.

In some embodiments of the foregoing methods, the light chain antibody variable region is an engineered light chain antibody variable region.

In some embodiments of the foregoing methods, the generation of the engineered light chain antibody variable region includes: providing information comprising: (i) an acceptor light chain antibody variable region amino acid sequence comprising the amino acid sequence depicted in SEQ ID NO:2 or SEQ ID NO:8 or (ii) a nucleic acid sequence encoding the acceptor light chain antibody variable region amino acid sequence; providing information comprising: (iii) at least one donor antibody light chain variable region amino acid sequence or (iv) a nucleic acid sequence encoding the donor antibody light chain variable region amino acid sequence; replacing one or more CDRs of the acceptor light chain antibody variable region with one or more CDRs from the donor antibody light chain variable region to thereby generate an engineered light chain variable region that is less immunogenic in a human as compared to the immunogenicity of the donor antibody light chain variable region, with the proviso that the engineered light chain variable region does not comprise a light chain polypeptide comprising the complete amino acid sequence depicted in SEQ ID NO:2 or SEQ ID NO:8.

In some embodiments, the methods include producing the engineered antibody light chain variable region and/or the engineered antibody heavy chain variable region (the light chain and heavy chain variable regions can, in some embodiments, contain constant regions as described herein). In some embodiments, the engineered antibody light chain variable region is produced in a cell or using a cell-free system.

In some embodiments, the methods include isolating from the cell or the media in which the cell is cultured the engineered antibody light chain variable region and/or the engineered heavy chain variable region.

In some embodiments, the methods include producing the engineered antibody. The engineered antibody can be produced in a cell or using a cell-free system. In some embodiments, the methods include isolating from the cell or the media in which the cell is cultured the engineered antibody.

In some embodiments, the methods include determining whether the engineered antibody binds to the same antigen as the donor antibody. In some embodiments, the engineered antibody can have a greater affinity for a target antigen as compared to the affinity of the donor antibody for the same antigen.

In some embodiments, the methods include determining whether an antibody that binds to the engineered antibody is produced after the engineered antibody is administered to a human.

In some embodiments, the methods include reshaping the engineered antibody. In some embodiments, the reshaping includes substituting at least one amino acid of a framework region. In some embodiments, the reshaping includes substituting at least two amino acids of a framework region. In some embodiments, the reshaping includes substituting at least one amino acid in at least two different framework regions. In some embodiments, the reshaping does not include substituting one or more amino acids in a framework region.

In some embodiments, the reshaping includes substituting at least one amino acid of at least one CDR. The reshaping, in some embodiments, include substituting at least two amino acids of at least one CDR. In some embodiments, the reshaping includes substituting at least one amino acid position of a CDR, wherein the CDR is defined according to Kabat or the combined Kabat-Chothia definition.

In some embodiments, the reshaping includes substituting amino acids at one or both of positions 28 and 30 (according to the Kabat numbering) of the heavy chain variable region. In some embodiments, the reshaping includes substituting at least one amino acid in at least two different CDRs. In some embodiments, the reshaping includes substituting at least one amino acid at position 27, 28, 30, 71, or 78 (according to the Kabat numbering) of the heavy chain variable region.

In some embodiments, the reshaping includes introducing at least one spacer amino acid sequence into one or both of a light chain variable region and a heavy chain variable region of the engineered antibody.

In some embodiments of the foregoing methods, one or more amino acids of a framework or a CDR are substituted prior to the replacing. In some embodiments, one or more amino acids of a framework or a CDR are substituted after the replacing.

In some embodiments, the acceptor antibody light chain variable region comprises the amino acid sequence of any of the light chain polypeptides described herein. In some embodiments, the acceptor antibody heavy chain variable region amino acid sequence comprises the amino acid sequence of any of the heavy chain polypeptides described herein. In some embodiments of the foregoing methods, the acceptor antibody light chain variable region amino acid sequence comprises the amino acid sequence of any of the light chain polypeptides described herein and the acceptor antibody heavy chain variable region amino acid sequence comprises the amino acid sequence of any of the heavy chain polypeptides described herein.

In yet another aspect, the disclosure features an engineered antibody comprising: (i) a light chain polypeptide and (ii) a heavy chain polypeptide, wherein the light chain polypeptide comprises the following amino acid sequence segments in order: LFR1-LCDR1-LFR2-LCDR2-LFR3-LCDR3-LFR4. In some embodiments, LFR1 comprises the amino acid sequence depicted in SEQ ID NO:9, but with zero to three amino acid substitutions; LFR2 comprises the amino acid sequence depicted in SEQ ID NO:10 or SEQ ID NO:18, but with zero to three amino acid substitutions; LFR3 comprises the amino acid sequence depicted in SEQ ID NO:11, but with zero to three amino acid substitutions; and LFR4 comprises the amino acid sequence depicted in SEQ ID NO:12, but with zero to three amino acid substitutions; LCDR1 comprises the amino acid sequence of light chain CDR1 from a donor antibody, LCDR2 comprises the amino acid sequence of light chain CDR2 from a donor antibody, and LCDR3 comprises the amino acid sequence of light chain CDR3 from a donor antibody. The light chain polypeptide does not comprise the amino acid sequence depicted in SEQ ID NO:2 or SEQ ID NO:8. In some embodiments, the heavy chain polypeptide comprises the following amino acid sequence segments in order: HFR1-HCDR1-HFR2-HCDR2-HFR3-HCDR3-HFR4, wherein HFR1 comprises the amino acid sequence depicted in SEQ ID NOs: 13, 17, or 19, but with zero to three amino acid substitutions; HFR2 comprises the amino acid sequence depicted in SEQ ID NO:14, but with zero to three amino acid substitutions; HFR3 comprises the amino acid sequence depicted in SEQ ID NO:15, but with zero to three amino acid substitutions; and HFR4 comprises the amino acid sequence depicted in SEQ ID NO:16, but with zero to three amino acid substitutions, wherein HCDR1 comprises the amino acid sequence of heavy chain CDR1 from a donor antibody, HCDR2 comprises the amino acid sequence of heavy chain CDR2 from a donor antibody, and HCDR3 comprises the amino acid sequence of heavy chain CDR3 from a donor antibody. The heavy chain polypeptide does not comprise the amino acid sequence depicted in SEQ ID NO:5 or SEQ ID NO:7. The engineered antibody is less immunogenic in a human as compared to the donor antibody or antibodies and the engineered antibody binds to the same antigen as the donor antibody or antibodies.

In some embodiments, one or both of: (a) the LCDR1, LCDR2, and LCDR3 are from a single donor antibody; and (b) the HCDR1, HCDR2, and HCDR3 are from a single donor antibody. In some embodiments, all of the light chain CDRs and heavy chain CDRs are from the same donor antibody.

Unless otherwise defined, all technical and scientific terms used herein have the same meaning as commonly understood by one of ordinary skill in the art to which this disclosure pertains. In case of conflict, the present document, including definitions, will control. Preferred methods and materials are described below, although methods and materials similar or equivalent to those described herein can also be used in the practice or testing of the presently disclosed methods and compositions. All publications, patent applications, patents, and other references mentioned herein are incorporated by reference in their entirety.

Other features and advantages of the present disclosure, e.g., methods for generating a therapeutic antibody with reduced immunogenicity in a human, will be apparent from the following description, the examples, and from the claims.

### BRIEF DESCRIPTION OF THE DRAWINGS

FIG. 1 depicts an alignment of the amino acid sequence for the light chain variable region of eculizumab (“Ecu”) (SEQ ID NO:2) and the amino acid sequence for the I.23 immunoglobulin light chain variable region (1.23″) (SEQ ID NO:8). The three complementarity determining regions (CDRs)—LCDR1, LCDR2, and LCDR3—as defined according to the Kabat and Chothia definitions are identified by bracketing. The light chain variable region framework regions are also indicated. The amino acid position (as defined by Kabat numbering) with respect to the eculizumab sequence is indicated above the aligned sequences.

FIG. 2 depicts an alignment of the amino acid sequence for the heavy chain variable region of eculizumab (“Ecu”) (SEQ ID NO:5) and the amino acid sequence for the H20C3 immunoglobulin heavy chain variable region (“H20C3”) (SEQ ID NO:7). The three complementarity determining regions (CDRs)—HCDR1, HCDR2, and HCDR3—as defined according to the Kabat and Chothia definitions are identified by bracketing. The heavy chain variable region framework regions are also indicated. The amino acid position (as defined by Kabat numbering) with respect to the eculizumab sequence is indicated above the aligned sequences. Positions 52a, 82a, 82b, 82c, 100a, 100b, 100c, 100d, and 100e are also specifically identified.

### DETAILED DESCRIPTION

The disclosure provides engineered antibodies that exhibit less immunogenicity in a human as compared to the immunogenicity of the respective donor antibodies from which the engineered antibodies were derived. While in no way intended to be limiting, exemplary compositions, as well as methods for their preparation and use are elaborated on below.

### Engineered Antibodies

As used herein, an “engineered antibody” is an antibody comprising one or more (e.g., two, three, four, five, or six) CDRs of a donor antibody grafted onto the variable regions of an acceptor antibody scaffold, wherein the engineered antibody has less immunogenicity in a human as compared to the immunogenicity of the donor antibody in a human. The structure of the engineered antibody is as follows.

The engineered antibodies comprise a light chain polypeptide having a sequence comprising, or consisting of, the following amino acid sequence segments in order: LFR1-LCDR1-LFR2-LCDR2-LFR3-LCDR3-LFR4. LFR1 corresponds to the amino acid sequence of framework 1 (FR1) of the light chain variable region; LFR2 corresponds to the amino acid sequence of framework 2 (FR2) of the light chain variable region; LFR3 corresponds to the amino acid sequence of framework 3 (FR3) of the light chain variable region; and LFR4 corresponds to the amino acid sequence of framework 4 (FR4) of the light chain variable region. LCDR1 corresponds to the amino acid sequence of the complementarity determining region 1 (CDR1) of the light chain variable region; LCDR2 corresponds to the amino acid sequence of the complementarity determining region 2 (CDR2) of the light chain variable region; and LCDR3 corresponds to the amino acid sequence of the complementarity determining region 3 (CDR3) of the light chain variable region. One or more (e.g., one, two, three, or all four) of LFR1, LFR2, LFR3, and LFR4 amino acid sequences of the engineered antibody are contributed by an acceptor antibody. In some embodiments, only LFR1, LFR2, or LFR3 are contributed by an acceptor antibody. In some embodiments, LFR1 and LFR2 are contributed by an acceptor antibody. In some embodiments, LFR2 and LFR3 are contributed by an acceptor antibody. In some embodiments, LFR1 and LFR3 are contributed by an acceptor antibody. In some embodiments, LFR4 is not contributed by an acceptor antibody. One or more of the LCDR1, LCDR2, and LCDR3 amino acid sequences are contributed from at least one (e.g., one, two, or three) donor antibody. For example, the light chain CDRs can be obtained from a single donor antibody or, in some embodiments, CDRs from two or more different donor antibodies (e.g., two antibodies that bind to the same antigen, but have different light chain CDR sequences). In some embodiments, at least one (e.g., one, two, or even all three) of the LCDRs is obtained from the acceptor antibody. For example, an engineered antibody can have a LCDR3 from a donor antibody and LCDR1 and LCDR2 from the acceptor antibody. In some embodiments, an engineered antibody can have a LCDR2 from a donor antibody and a LCDR1 and LCDR3 from the acceptor antibody. Suitable acceptor antibodies and donor antibodies are elaborated on herein.

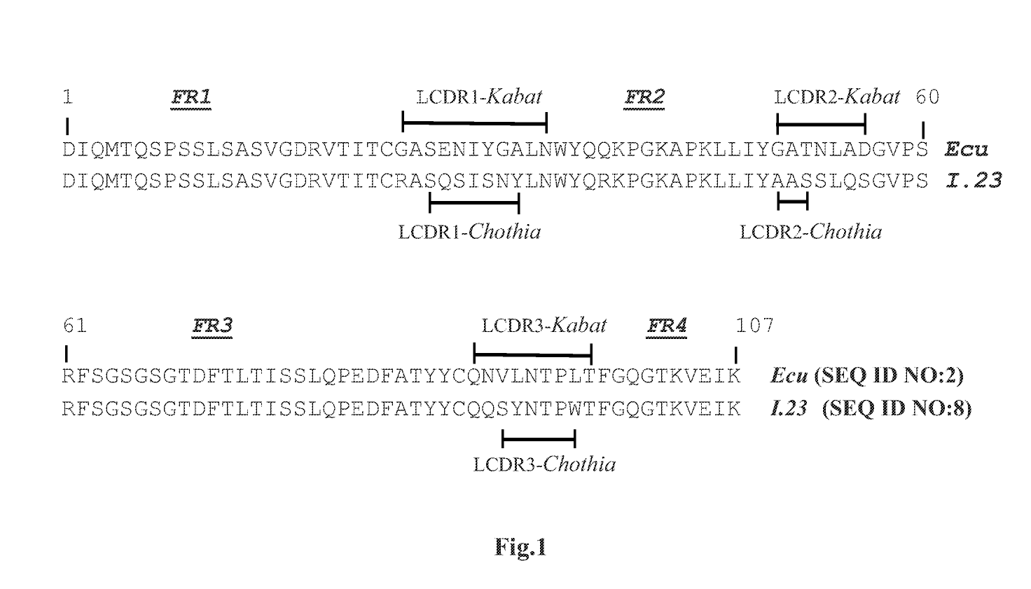

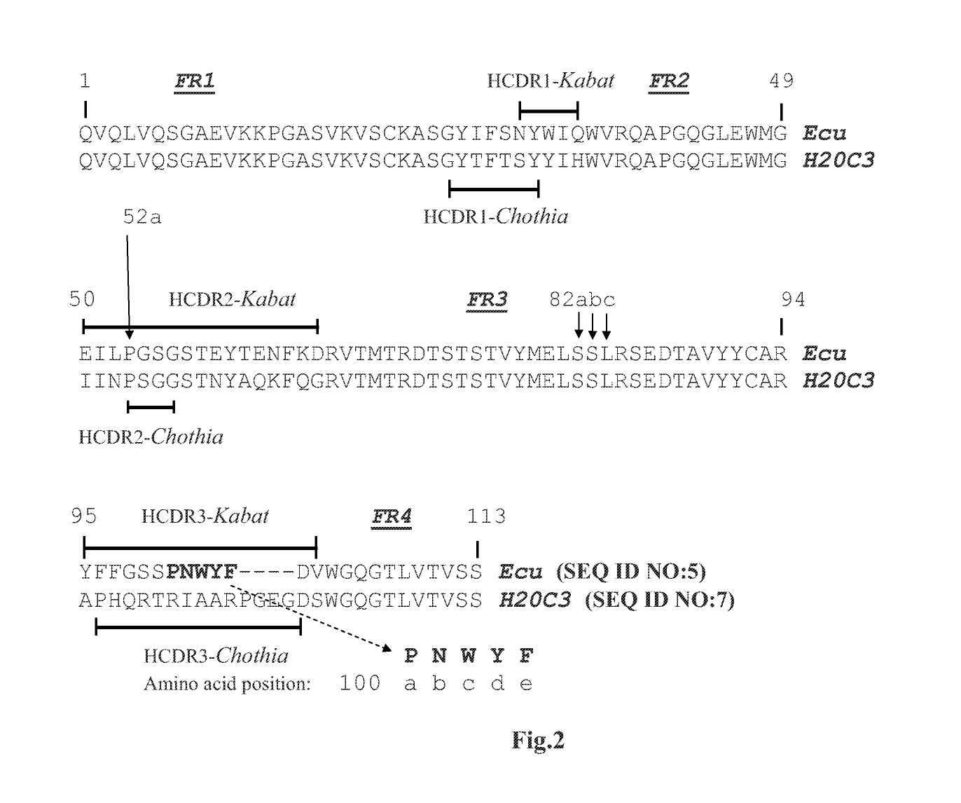

The exact boundaries of CDRs and framework regions have been defined differently according to different methods. In some embodiments, the positions of the CDRs or framework regions within a light or heavy chain variable domain can be as defined by Kabat et al. [(1991) “Sequences of Proteins of Immunological Interest.” NIH Publication No. 91-3242, U.S. Department of Health and Human Services, Bethesda, Md.]. In such cases, the CDRs can be referred to as “Kabat CDRs” (e.g., “Kabat LCDR2” or “Kabat HCDR1”) and the framework regions can be referred to as “Kabat framework regions,” (e.g., “Kabat LFR1” or “Kabat HFR3”). In some embodiments, the positions of the CDRs or framework regions of a light or heavy chain variable region can be as defined by Chothia et al. (1989) Nature 342:877-883. Accordingly, these regions can be referred to as “Chothia CDRs” (e.g., “Chothia LCDR2” or “Chothia HCDR3”) or “Chothia framework regions” (e.g., “Chothia LFR1” or “Chothia LFR3”), respectively. In some embodiments, the positions of the CDRs or framework regions of the light and heavy chain variable regions can be as defined by a Kabat-Chothia combined definition. In such embodiments, these regions can be referred to as “combined Kabat-Chothia CDRs” or “combined Kabat-Chothia framework regions,” respectively. Thomas et al. [(1996) Mol Immunol 33(17/18):1389-1401] exemplifies the identification of CDRs and framework region boundaries according to Kabat and Chothia definitions. The identification of CDRs and frameworks using each of the three aforementioned definitions is also shown in FIGS. 1 and 2.

In some embodiments, the positions of the CDRs and/or framework regions with a light or heavy chain variable domain can be as defined by Honnegger and Plückthun [(2001) J Mol Biol 309: 657-670].

As described herein and exemplified in the working Examples, the light chain variable region of eculizumab was generated by grafting the LCDRs of a murine anti-C5 antibody onto the framework region scaffold of the I.23 Ig kappa light chain molecule. The amino acid sequence of the light chain variable region of eculizumab is as follows: DIQMTQSPSSLSASVGDRVTITCGASENIYGALNWYQQKPGKAPKLLIYGATN LADGVPSRFSGSGSGTDFTLTISSLQPEDFATYYCQNVLNTPLTFGQGTKVEIK (SEQ ID NO:2). The amino acid sequence of the light chain variable region of I.23 is as follows: DIQMTQSPSSLSASVGDRVTITCRASQSISNYLNWYQRKPGKAPK LLIYAASSLQSGVPSRFSGSGSGTDFTLTISSLQPEDFATYYCQQSYNTPWTFG QGTKVEIK (SEQ ID NO:8). The LFR2 amino acid sequence of eculizumab differs from the amino acid sequence of the corresponding I.23 LFR2 by one amino acid: a glutamine at position 38 instead of an arginine.

Without being bound to any particular theory or mechanism of action, it is believed that the light chain framework region sequences (i.e., LFR1, LFR2, LFR3, and/or LFR4) from either eculizumab or I.23 would be useful in the preparation of an engineered antibody that exhibits a reduced level of immunogenicity in a human as compared to the level of immunogenicity of a donor antibody. Thus, in some embodiments, LFR1, LFR2, LFR3, and/or LFR4 can be the corresponding light chain framework regions derived from eculizumab and/or I.23. Amino acid sequences for the eculizumab and I.23 light chain framework regions, as defined by Kabat, Chothia, or Kabat-Chothia, are set forth in Table 1.

TABLE 1
Exemplary Acceptor Antibody Light Chain Framework Regions.
Confirmation FA Requested Search Report? : Sep 4, 2010

| | Framework | | Source | SEQ |
|---|---|---|---|---|
| Amino Acid Sequence | Region (FR) | Definition | Sequence | ID NO: |
| DIQMTQSPSSLSASVGDRVTITC | Light | Kabat | eculizumab | 9 |
| | Chain, FR1 | | | |
| WYQQKPGKAPKLLIY | Light | Kabat | eculizumab | 10 |
| | Chain, FR2 | | | |
| GVPSRFSGSGSGTDFTLTISSLQPEDFATYYC | Light | Kabat | eculizumab | 11 |
| | Chain, FR3 | | | |
| FGQGTKVEIK | Light | Kabat | eculizumab | 12 |
| | Chain, FR4 | | | |
| DIQMTQSPSSLSASVGDRVTITCGA | Light | Chothia | eculizumab | 20 |
| | Chain, FR1 | | | |
| LNWYQQKPGKAPKLLIY | Light | Chothia | eculizumab | 21 |
| | Chain, FR2 | | | |
| NLADGVPSRFSGSGSGTDFTLTISSLQPEDFATYYCQN | Light | Chothia | eculizumab | 22 |
| | Chain, FR3 | | | |
| TFGQGTKVEIK | Light | Chothia | eculizumab | 23 |
| | Chain, FR4 | | | |
| DIQMTQSPSSLSASVGDRVTITC | Light | Kabat- | eculizumab | 9 |
| | Chain, FR1 | Chothia | | |
| WYQQKPGKAPKLLIY | Light | Kabat- | eculizumab | 10 |
| | Chain, FR2 | Chothia | | |
| GVPSRFSGSGSGTDFTLTISSLQPEDFATYYC | Light | Kabat- | eculizumab | 11 |
| | Chain, FR3 | Chothia | | |
| FGQGTKVEIK | Light | Kabat- | eculizumab | 12 |
| | Chain, FR4 | Chothia | | |
| DIQMTQSPSSLSASVGDRVTITC | Light | Kabat | I.23 | 9 |
| | Chain, FR1 | | | |
| WYQRKPGKAPKLLIY | Light | Kabat | I.23 | 18 |
| | Chain, FR2 | | | |
| GVPSRFSGSGSGTDFTLTISSLQPEDFATYYC | Light | Kabat | I.23 | 11 |
| | Chain, FR3 | | | |
| FGQGTKVEIK | Light | Kabat | I.23 | 12 |
| | Chain, FR4 | | | |
| DIQMTQSPSSLSASVGDRVTITCRA | Light | Chothia | I.23 | 24 |
| | Chain, FR1 | | | |
| LNWYQRKPGKAPKLLIY | Light | Chothia | I.23 | 25 |
| | Chain, FR2 | | | |
| SLQSGVPSRFSGSGSGTDFTLTISSLQPEDFATYYCQQ | Light | Chothia | I.23 | 26 |
| | Chain, FR3 | | | |
| TFGQGTKVEIK | Light | Chothia | I.23 | 23 |
| | Chain, FR4 | | | |
| DIQMTQSPSSLSASVGDRVTITC | Light | Kabat- | I.23 | 9 |
| | Chain, FR1 | Chothia | | |
| WYQRKPGKAPKLLIY | Light | Kabat- | I.23 | 18 |
| | Chain, FR2 | Chothia | | |
| GVPSRFSGSGSGTDFTLTISSLQPEDFATYYC | Light | Kabat- | I.23 | 11 |
| | Chain, FR3 | Chothia | | |
| FGQGTKVEIK | Light | Kabat- | I.23 | 12 |
| | Chain, FR4 | Chothia | | |

Thus, in some embodiments, the light chain polypeptide of the engineered antibody comprises, or consists of: an LFR1 element comprising, or consisting of, the amino acid sequence depicted in SEQ ID NO:9; an LFR2 element comprising, or consisting of, the amino acid sequence depicted in SEQ ID NO:10; an LFR3 element comprising, or consisting of, the amino acid sequence depicted in SEQ ID NO:11; and an LFR4 element comprising, or consisting of, the amino acid sequence depicted in SEQ ID NO:12.

In some embodiments, the light chain polypeptide of the engineered antibody comprises, or consists of: an LFR1 element comprising, or consisting of, the amino acid sequence depicted in SEQ ID NO:9; an LFR2 element comprising, or consisting of, the amino acid sequence depicted in SEQ ID NO:18; an LFR3 element comprising, or consisting of, the amino acid sequence depicted in SEQ ID NO:11; and an LFR4 element comprising, or consisting of, the amino acid sequence depicted in SEQ ID NO:12.

In some embodiments, the light chain polypeptide of the engineered antibody comprises, or consists of: an LFR1 element comprising, or consisting of, the amino acid sequence depicted in SEQ ID NO:20; an LFR2 element comprising, or consisting of, the amino acid sequence depicted in SEQ ID NO:21; an LFR3 element comprising, or consisting of, the amino acid sequence depicted in SEQ ID NO:22; and an LFR4 element comprising, or consisting of, the amino acid sequence depicted in SEQ ID NO:23.

In some embodiments, the light chain polypeptide of the engineered antibody comprises, or consists of: an LFR1 element comprising, or consisting of, the amino acid sequence depicted in SEQ ID NO:24; an LFR2 element comprising, or consisting of, the amino acid sequence depicted in SEQ ID NO:25; an LFR3 element comprising, or consisting of, the amino acid sequence depicted in SEQ ID NO:26; and an LFR4 element comprising, or consisting of, the amino acid sequence depicted in SEQ ID NO:23.

In some embodiments, the light chain polypeptide does not comprise or consist of the amino acid sequence depicted in SEQ ID NO:2 or SEQ ID NO:8.

The light chain polypeptide can comprise a constant region. For example, the light chain constant region can be a λ light chain polypeptide constant region or a κ light chain constant region. The amino acid sequence for a number of human λ and κ light chain constant regions are known in the art and described in, e.g., Kabat et al. (1991); supra). The light chain polypeptide of the engineered antibody can comprise a light chain constant region having the following amino acid sequence: RTVAAPSVFIFPPSDEQLKSGTASVVCLLNNFYPREAKVQWKVDNALQSGNS QESVTEQDSKDSTYSLSSTLTLSKADYEKHKVYACEVTHQGLSSPVTKSFNRG EC (SEQ ID NO:3). SEQ ID NO:3 is the constant region of the light chain of eculizumab.

The engineered antibodies described herein also comprise a heavy chain polypeptide having an amino acid sequence that comprises or consists of the following amino acid sequence segments in order: HFR1-HCDR1-HFR2-HCDR2-HFR3-HCDR3-HFR4. HFR1 corresponds to the amino acid sequence of framework 1 (FR1) of the heavy chain variable region; HFR2 corresponds to the amino acid sequence of framework 2 (FR2) of the heavy chain variable region; HFR3 corresponds to the amino acid sequence of framework 3 (FR3) of the heavy chain variable region; and HFR4 corresponds to the amino acid sequence of framework 4 (FR4) of the heavy chain variable region. HCDR1 corresponds to the amino acid sequence of the complementarity determining region 1 (CDR1) of the heavy chain variable region; HCDR2 corresponds to the amino acid sequence of the complementarity determining region 2 (CDR2) of the heavy chain variable region; and HCDR3 corresponds to the amino acid sequence of the complementarity determining region 3 (CDR3) of the heavy chain variable region. The HFR1, HFR2, HFR3, and HFR4 amino acid sequences are contributed from an acceptor antibody, whereas the HCDR1, HCDR2, and HCDR3 amino acid sequences can be contributed from at least one (e.g., one, two, or three) donor antibody. For example, the heavy chain CDRs can be obtained from a single donor antibody or, in some embodiments, CDRs from two or more different donor antibodies (e.g., two antibodies that bind to the same antigen, but have different heavy chain CDR sequences). In some embodiments, at least one of the HCDRs is retained (or contributed) from the acceptor antibody. For example, HCDR3 can be from a donor antibody (e.g., where HCDR3 has been determined to contribute to the donor antibody the most binding energy for the antigen to which the donor antibody binds) and HCDR1 and HCDR2 can be retained from the acceptor antibody. In some embodiments, HCDR1, HCDR2, and HCDR3 are each contributed from a single donor antibody. Suitable acceptor antibodies and donor antibodies are elaborated on herein.

As described herein and exemplified in the working examples, the heavy chain variable region of eculizumab was generated by grafting the HCDRs of a murine anti-C5 antibody onto the heavy chain framework region scaffold of the H20C3 Ig molecule. The amino acid sequence of the heavy chain variable region of eculizumab is as follows: QVQLVQSGAEVKKPGASVKVSCKASGYIFSNYWIQWVRQAPGQGLEWMGEI LPGSGSTEYTENFKDRVTMTRDTSTSTVYMELSSLRSEDTAVYYCARYFFGSS PNWYFDVWGQGTLVTVSS (SEQ ID NO:5). The amino acid sequence of the heavy chain variable region of H20C3 is as follows: QVQLVQSGAEVKKPGASVKVSCKASGYTFTSYYIHWVRQAPGQGLEWMGII NPSGGSTNYAQKFQGRVTMTRDTSTSTVYMELSSLRSEDTAVYYCARAPHQ RTRIAARPGEGDSWGQGTLVTVSS (SEQ ID NO:7). As defined by Kabat, the amino acid sequence of the HFR1 of eculizumab differs from the corresponding amino acid sequence of HFR1 of H20C3 by two amino acids. Specifically, the threonine at position 28 and the threonine at position 30 of the H20C3 V_{H} region (Kabat FR1) are isoleucine and serine, respectively, in the eculizumab Kabat FR1 sequence. The remaining framework regions (HFR2, HFR3, and HFR4) are identical between eculizumab and H20C3 under the Kabat definition. Under the combined Kabat-Chothia definition, there is no difference between the amino acid sequence of eculizumab and the amino acid sequence of H20C3 for any of the framework regions. (See FIG. 2.)

Without being bound to any particular theory or mechanism of action, it is believed that heavy chain framework region sequences from either eculizumab or H20C3 will be useful in the preparation of an engineered antibody that exhibits a reduced level of immunogenicity in a human as compared to the level of immunogenicity of a donor antibody. Thus, in some embodiments, HFR1, HFR2, HFR3, and/or HFR4 can be the corresponding heavy chain framework regions derived from eculizumab and/or H20C3. Amino acid sequences for the eculizumab and H20C3 heavy chain framework regions, as defined by Kabat, Chothia, or Kabat-Chothia, are set forth in Table 2.

TABLE 2
Exemplary Acceptor Antibody Heavy Chain Framework Regions.

| | Framework | | Source | SEQ |
|---|---|---|---|---|
| Amino Acid Sequence | Region (FR) | Definition | Sequence | ID NO: |
| QVQLVQSGAEVKKPGASVKVSCKASGYIFS | Heavy | Kabat | eculizumab | 13 |
| | Chain, FR1 | | | |
| WVRQAPGQGLEWMG | Heavy | Kabat | eculizumab | 14 |
| | Chain, FR2 | | | |
| RVTMTRDTSTSTVYMELSSLRSED | Heavy | Kabat | eculizumab | 15 |
| TAVYYCAR | Chain, FR3 | | | |
| WGQGTLVTVSS | Heavy | Kabat | eculizumab | 16 |
| | Chain, FR4 | | | |
| QVQLVQSGAEVKKPGASVKVSCKAS | Heavy | Chothia | eculizumab | 17 |
| | Chain, FR1 | | | |
| WIQWVRQAPGQGLEWMGEIL | Heavy | Chothia | eculizumab | 27 |
| | Chain, FR2 | | | |
| STEYTENFKDRVTMTRDTSTSTVYME | Heavy | Chothia | eculizumab | 28 |
| LSSLRSEDTAVYYCARY | Chain, FR3 | | | |
| VWGQGTLVTVSS | Heavy | Chothia | eculizumab | 29 |
| | Chain, FR4 | | | |
| WVRQAPGQGLEWMG | Heavy | Kabat- | eculizumab | 14 |
| | Chain, FR2 | Chothia | | |
| RVTMTRDTSTSTVYMELSSLRSED | Heavy | Kabat- | eculizumab | 15 |
| TAVYYCAR | Chain, FR3 | Chothia | | |
| WGQGTLVTVSS | Heavy | Kabat- | eculizumab | 16 |
| | Chain, FR4 | Chothia | | |
| QVQLVQSGAEVKKPGASVKVSCKASGYTFT | Heavy | Kabat | H20C3 | 19 |
| | Chain, FR1 | | | |
| WVRQAPGQGLEWMG | Heavy | Kabat | H20C3 | 14 |
| | Chain, FR2 | | | |
| RVTMTRDTSTSTVYMELSSLRSEDTAV | Heavy | Kabat | H20C3 | 15 |
| YYCAR | Chain, FR3 | | | |
| WGQGTLVTVSS | Heavy | Kabat | H20C3 | 16 |
| | Chain, FR4 | | | |
| QVQLVQSGAEVKKPGASVKVSCKAS | Heavy | Chothia | H20C3 | 17 |
| | Chain, FR1 | | | |
| YIHWVRQAPGQGLEWMGIIN | Heavy | Chothia | H20C3 | 30 |
| | Chain, FR2 | | | |
| STNYAQKFQGRVTMTRDTSTSTVYME | Heavy | Chothia | H20C3 | 31 |
| LSSLR-SEDTAVYYCARA | Chain, FR3 | | | |
| SWGQGTLVTVSS | Heavy | Chothia | H20C3 | 32 |
| | Chain, FR4 | | | |
| QVQLVQSGAEVKKPGASVKVSCKAS | Heavy | Kabat- | H20C3 | 17 |
| | Chain, FR1 | Chothia | | |
| WVRQAPGQGLEWMG | Heavy | Kabat- | H20C3 | 14 |
| | Chain, FR2 | Chothia | | |
| RVTMTRDTSTSTVYMELSSLRSEDT | Heavy | Kabat- | H20C3 | 15 |
| AVYYCAR | Chain, FR3 | Chothia | | |
| WGQGTLVTVSS | Heavy | Kabat- | H20C3 | 16 |
| | Chain, FR4 | Chothia | | |

Thus, in some embodiments HFR1 of the engineered antibody can comprise or be, e.g., the amino acid sequence depicted in SEQ ID NOs: 13, 17, or 19. In some embodiments, HFR2 of the engineered antibody can comprise or be, e.g., the amino acid sequence depicted in SEQ ID NO: 14. In some embodiments, HFR3 of the engineered antibody can comprise or be, e.g., the amino acid sequence depicted in SEQ ID NO:15. In some embodiments, HFR4 of the engineered antibody can comprise or be, e.g., the amino acid sequence depicted in SEQ ID NO:16.

In some embodiments, the heavy chain polypeptide of the engineered antibody comprises, or consists of: an HFR1 element comprising, or consisting of, the amino acid sequence depicted in SEQ ID NO:13; an HFR2 element comprising, or consisting of, the amino acid sequence depicted in SEQ ID NO:14; an HFR3 element comprising, or consisting of, the amino acid sequence depicted in SEQ ID NO:15; and an HFR4 element comprising, or consisting of, the amino acid sequence depicted in SEQ ID NO:16.

In some embodiments, the heavy chain polypeptide of the engineered antibody comprises, or consists of: an HFR1 element comprising, or consisting of, the amino acid sequence depicted in SEQ ID NO:19; an HFR2 element comprising, or consisting of, the amino acid sequence depicted in SEQ ID NO:14; an HFR3 element comprising, or consisting of, the amino acid sequence depicted in SEQ ID NO:15; and an HFR4 element comprising, or consisting of, the amino acid sequence depicted in SEQ ID NO:16.

In some embodiments, the heavy chain polypeptide of the engineered antibody comprises, or consists of: an HFR1 element comprising, or consisting of, the amino acid sequence depicted in SEQ ID NO:17; an HFR2 element comprising, or consisting of, the amino acid sequence depicted in SEQ ID NO:14; an HFR3 element comprising, or consisting of, the amino acid sequence depicted in SEQ ID NO:15; and an HFR4 element comprising, or consisting of, the amino acid sequence depicted in SEQ ID NO:16.

In some embodiments, the heavy chain polypeptide of the engineered antibody comprises, or consists of: an HFR1 element comprising, or consisting of, the amino acid sequence depicted in SEQ ID NO:17; an HFR2 element comprising, or consisting of, the amino acid sequence depicted in SEQ ID NO:27; an HFR3 element comprising, or consisting of, the amino acid sequence depicted in SEQ ID NO:28; and an HFR4 element comprising, or consisting of, the amino acid sequence depicted in SEQ ID NO:29.

In some embodiments, the heavy chain polypeptide of the engineered antibody comprises, or consists of: an HFR1 element comprising, or consisting of, the amino acid sequence depicted in SEQ ID NO:17; an HFR2 element comprising, or consisting of, the amino acid sequence depicted in SEQ ID NO:30; an HFR3 element comprising, or consisting of, the amino acid sequence depicted in SEQ ID NO:31; and an HFR4 element comprising, or consisting of, the amino acid sequence depicted in SEQ ID NO:32.

In some embodiments, the heavy chain polypeptide does not comprise or consist of the amino acid sequence depicted in SEQ ID NO:5 or SEQ ID NO:7.

The heavy chain polypeptide can comprise a constant region (e.g., a heavy chain constant region 1 (CH1), heavy chain constant region 2 (CH2), heavy chain constant region 3 (CH3), a heavy chain constant region 4 (CH4), or a combination of any of the foregoing). The heavy chain polypeptide can comprise an Fc portion of an immunoglobulin molecule. The Fc region can be, e.g., an Fc region from an IgG1, IgG2, IgG3, IgG4, IgA, IgM, IgE, or IgD immunoglobulin molecule or a combination of portions of each of these. The amino acid sequences for a number of human heavy chain constant regions are known in the art and described in, e.g., Kabat et al. (1991), supra.

In some embodiments, the heavy chain polypeptide can comprise a hybrid constant region, or a portion thereof, such as a G2/G4 hybrid constant region (see e.g., Burton et al. (1992) Adv Immun. 51:1-18; Canfield et al. (1991) J Exp Med 173:1483-1491; and Mueller et al. (1997) Mol. Immunol. 34(6):441-452). For example (and in accordance with Kabat numbering), the IgG1 and IgG4 constant regions comprise G₂₄₉G₂₅₀ residues whereas the IgG2 constant region does not comprise residue 249, but does comprise G₂₅₀. In a G2/G4 hybrid constant region, where the 249-250 region comes from the G2 sequence, the constant region can be further modified to introduce a glycine residue at position 249 to produce a G2/G4 fusion having G₂₄₉/G₂₅₀. Other constant domain hybrids that comprise G₂₄₉/G₂₅₀ can also be part of engineered antibodies in accordance with the disclosure.

In some embodiments, the heavy chain polypeptide comprises a constant region comprising, or consisting of, the following amino acid sequence: ASTKGPSVFPLAPCSRSTSESTAALGCLVKDYFPEPVTVSWNSGALTSGVHTF PAVLQSSGLYSLSSVVTVPSSNFGTQTYTCNVDHKPSNTKVDKTVERKCCVE CPPCPAPPVAGPSVFLFPPKPKDTLMISRTPEVTCVVVDVSQEDPEVQFNWYV DGVEVHNAKTKPREEQFNSTYRVVSVLTVLHQDWLNGKEYKCKVSNKGLPS SIEKTISKAKGQPREPQVYTLPPSQEEMTKNQVSLTCLVKGFYPSDIAVEWES NGQPENNYKTTPPVLDSDGSFFLYSRLTVDKSRWQEGNVFSCSVMHEALHN HYTQKSLSLSLGK (SEQ ID NO:6). SEQ ID NO:6 depicts the amino acid sequence of the heavy chain constant region of eculizumab.

The engineered antibodies described herein can, in some embodiments, comprise particular exemplary pairings of light chain framework regions and heavy chain framework regions. For example, an engineered antibody described herein can comprise a light chain polypeptide comprising the “Kabat” light chain framework regions derived from eculizumab and a heavy chain polypeptide comprising the “Kabat” heavy chain framework regions derived from eculizumab. In another example, an engineered antibody described herein can comprise a light chain polypeptide comprising the “Kabat-Chothia” light chain framework regions derived from the I.23 light chain and a heavy chain polypeptide comprising the “Kabat-Chothia” heavy chain framework regions derived from the H20C3 heavy chain. In yet another example, an engineered antibody described herein can comprise a light chain polypeptide comprising the “Kabat” light chain framework regions derived from the I.23 light chain and a heavy chain polypeptide comprising the “Kabat” heavy chain framework regions derived from eculizumab. Exemplary pairings of light chain and heavy chain framework regions, the regions being defined under Kabat or the Kabat-Chothia combined definition, for use in the preparation of an engineered antibody are set forth in Table 3.

TABLE 3
Exemplary Heavy and Light Chain Framework Region Pairings.

| Pairing | | Definition | | | |
|---|---|---|---|---|---|
| Combination: | | Kabat | | Kabat-Chothia | |
| Heavy | | Light | Heavy | Light | Heavy |
| Chain/Light | Framework | Chain | Chain | Chain | Chain |
| Chain | Region | Sequence | Sequence | Sequence | Sequence |
| Ecu Light | FR1 | SEQ ID: 9 | SEQ ID: 13 | SEQ ID: 9 | SEQ ID: 17 |
| Chain/Ecu | FR2 | SEQ ID: 10 | SEQ ID: 14 | SEQ ID: 10 | SEQ ID: 14 |
| Heavy Chain | FR3 | SEQ ID: 11 | SEQ ID: 15 | SEQ ID: 11 | SEQ ID: 15 |
| | FR4 | SEQ ID: 12 | SEQ ID: 16 | SEQ ID: 12 | SEQ ID: 16 |
| I.23 Light | FR1 | SEQ ID: 9 | SEQ ID: 19 | SEQ ID: 9 | SEQ ID: 17 |
| Chain/H20C3 | FR2 | SEQ ID: 18 | SEQ ID: 14 | SEQ ID: 18 | SEQ ID: 14 |
| Heavy Chain | FR3 | SEQ ID: 11 | SEQ ID: 15 | SEQ ID: 11 | SEQ ID: 15 |
| | FR4 | SEQ ID: 12 | SEQ ID: 16 | SEQ ID: 12 | SEQ ID: 16 |
| I.23 Light | FR1 | SEQ ID: 9 | SEQ ID: 13 | SEQ ID: 9 | SEQ ID: 17 |
| Chain/Ecu | FR2 | SEQ ID: 18 | SEQ ID: 14 | SEQ ID: 18 | SEQ ID: 14 |
| Heavy Chain | FR3 | SEQ ID: 11 | SEQ ID: 15 | SEQ ID: 11 | SEQ ID: 15 |
| | FR4 | SEQ ID: 12 | SEQ ID: 16 | SEQ ID: 12 | SEQ ID: 16 |
| Ecu Light | FR1 | SEQ ID: 9 | SEQ ID: 19 | SEQ ID: 9 | SEQ ID: 17 |
| Chain/H20C3 | FR2 | SEQ ID: 10 | SEQ ID: 14 | SEQ ID: 10 | SEQ ID: 14 |
| Heavy Chain | FR3 | SEQ ID: 11 | SEQ ID: 15 | SEQ ID: 11 | SEQ ID: 15 |
| | FR4 | SEQ ID: 12 | SEQ ID: 16 | SEQ ID: 12 | SEQ ID: 16 |

* “SEQ ID” recited in Table 3 refers to “SEQ ID NO.”

Exemplary pairings of light chain and heavy chain framework regions, the regions being defined under Chothia, for use in the preparation of an engineered antibody are set forth in Table 4.

TABLE 4
Exemplary Heavy and Light Chain Framework Region Pairings,
the Regions being Defined by the Chothia Method (Part I).

| Pairing | | | |
|---|---|---|---|
| Combination: | | | |
| Heavy | | | |
| Chain/Light | Framework | Light Chain | Heavy Chain |
| Chain | Region | Sequence | Sequence |
| Ecu Light | FR1 | SEQ ID NO: 20 | SEQ ID NO: 17 |
| Chain/Ecu | FR2 | SEQ ID NO: 21 | SEQ ID NO: 27 |
| Heavy Chain | FR3 | SEQ ID NO: 22 | SEQ ID NO: 28 |
| | FR4 | SEQ ID NO: 23 | SEQ ID NO: 29 |
| Ecu Light | FR1 | SEQ ID NO: 20 | SEQ ID NO: 17 |
| Chain/H20C3 | FR2 | SEQ ID NO: 21 | SEQ ID NO: 30 |
| Heavy Chain | FR3 | SEQ ID NO: 22 | SEQ ID NO: 31 |
| | FR4 | SEQ ID NO: 23 | SEQ ID NO: 32 |
| I.23 Light | FR1 | SEQ ID NO: 24 | SEQ ID NO: 17 |
| Chain/Ecu | FR2 | SEQ ID NO: 25 | SEQ ID NO: 27 |
| Heavy Chain | FR3 | SEQ ID NO: 26 | SEQ ID NO: 28 |
| | FR4 | SEQ ID NO: 23 | SEQ ID NO: 29 |
| I.23 Light | FR1 | SEQ ID NO: 24 | SEQ ID NO: 17 |
| Chain/H20C3 | FR2 | SEQ ID NO: 25 | SEQ ID NO: 30 |
| Heavy Chain | FR3 | SEQ ID NO: 26 | SEQ ID NO: 31 |
| | FR4 | SEQ ID NO: 23 | SEQ ID NO: 32 |

Additional exemplary pairings of light chain and heavy chain framework regions, the regions being defined under Chothia, for use in the preparation of an engineered antibody are set forth in Table 5.

TABLE 5
Exemplary Heavy and Light Chain Framework Region Pairings,
the Regions being Defined by the Chothia Method (Part II).

| | Source | | | | |
|---|---|---|---|---|---|
| | Sequence | | Frame- | | |
| | Light | Heavy | work | Light Chain | Heavy Chain |
| | Chain | Chain | Region | Sequence | Sequence |
| A | Ecu | Ecu* | FR1 | SEQ ID NO: 20 | SEQ ID NO: 17 |
| | I.23 | Ecu | FR2 | SEQ ID NO: 25 | SEQ ID NO: 27 |
| | Ecu | Ecu | FR3 | SEQ ID NO: 22 | SEQ ID NO: 28 |
| | Ecu** | Ecu | FR4 | SEQ ID NO: 23 | SEQ ID NO: 29 |
| B | Ecu | Ecu* | FR1 | SEQ ID NO: 20 | SEQ ID NO: 17 |
| | I.23 | H20C3 | FR2 | SEQ ID NO: 25 | SEQ ID NO: 30 |
| | Ecu | H20C3 | FR3 | SEQ ID NO: 22 | SEQ ID NO: 31 |
| | Ecu** | H20C3 | FR4 | SEQ ID NO: 23 | SEQ ID NO: 32 |
| C | Ecu | Ecu* | FR1 | SEQ ID NO: 20 | SEQ ID NO: 17 |
| | I.23 | H20C3 | FR2 | SEQ ID NO: 25 | SEQ ID NO: 30 |
| | Ecu | Ecu | FR3 | SEQ ID NO: 22 | SEQ ID NO: 28 |
| | Ecu** | Ecu | FR4 | SEQ ID NO: 23 | SEQ ID NO: 29 |
| D | Ecu | Ecu* | FR1 | SEQ ID NO: 20 | SEQ ID NO: 17 |
| | I.23 | Ecu | FR2 | SEQ ID NO: 25 | SEQ ID NO: 27 |
| | Ecu | H20C3 | FR3 | SEQ ID NO: 22 | SEQ ID NO: 31 |
| | Ecu** | Ecu | FR4 | SEQ ID NO: 23 | SEQ ID NO: 29 |
| E | Ecu | Ecu* | FR1 | SEQ ID NO: 20 | SEQ ID NO: 17 |
| | I.23 | Ecu | FR2 | SEQ ID NO: 25 | SEQ ID NO: 27 |
| | Ecu | Ecu | FR3 | SEQ ID NO: 22 | SEQ ID NO: 28 |
| | Ecu** | H20C3 | FR4 | SEQ ID NO: 23 | SEQ ID NO: 32 |
| F | Ecu | Ecu* | FR1 | SEQ ID NO: 20 | SEQ ID NO: 17 |
| | I.23 | H20C3 | FR2 | SEQ ID NO: 25 | SEQ ID NO: 30 |
| | Ecu | Ecu | FR3 | SEQ ID NO: 22 | SEQ ID NO: 28 |
| | Ecu** | H20C3 | FR4 | SEQ ID NO: 23 | SEQ ID NO: 32 |
| G | Ecu | Ecu* | FR1 | SEQ ID NO: 20 | SEQ ID NO: 17 |
| | I.23 | H20C3 | FR2 | SEQ ID NO: 25 | SEQ ID NO: 30 |
| | Ecu | H20C3 | FR3 | SEQ ID NO: 22 | SEQ ID NO: 31 |
| | Ecu** | Ecu | FR4 | SEQ ID NO: 23 | SEQ ID NO: 29 |
| H | Ecu | Ecu* | FR1 | SEQ ID NO: 20 | SEQ ID NO: 17 |
| | I.23 | Ecu | FR2 | SEQ ID NO: 25 | SEQ ID NO: 27 |
| | Ecu | H20C3 | FR3 | SEQ ID NO: 22 | SEQ ID NO: 31 |
| | Ecu** | H20C3 | FR4 | SEQ ID NO: 23 | SEQ ID NO: 32 |
| I | Ecu | Ecu* | FR1 | SEQ ID NO: 20 | SEQ ID NO: 17 |
| | Ecu | Ecu | FR2 | SEQ ID NO: 21 | SEQ ID NO: 27 |
| | I.23 | Ecu | FR3 | SEQ ID NO: 26 | SEQ ID NO: 28 |
| | Ecu** | Ecu | FR4 | SEQ ID NO: 23 | SEQ ID NO: 29 |
| J | Ecu | Ecu* | FR1 | SEQ ID NO: 20 | SEQ ID NO: 17 |
| | Ecu | H20C3 | FR2 | SEQ ID NO: 21 | SEQ ID NO: 30 |
| | I.23 | H20C3 | FR3 | SEQ ID NO: 26 | SEQ ID NO: 31 |
| | Ecu** | H20C3 | FR4 | SEQ ID NO: 23 | SEQ ID NO: 32 |
| K | Ecu | Ecu* | FR1 | SEQ ID NO: 20 | SEQ ID NO: 17 |
| | Ecu | H20C3 | FR2 | SEQ ID NO: 21 | SEQ ID NO: 30 |
| | I.23 | Ecu | FR3 | SEQ ID NO: 26 | SEQ ID NO: 28 |
| | Ecu** | Ecu | FR4 | SEQ ID NO: 23 | SEQ ID NO: 29 |
| L | Ecu | Ecu* | FR1 | SEQ ID NO: 20 | SEQ ID NO: 17 |
| | Ecu | Ecu | FR2 | SEQ ID NO: 21 | SEQ ID NO: 27 |
| | I.23 | H20C3 | FR3 | SEQ ID NO: 26 | SEQ ID NO: 31 |
| | Ecu** | Ecu | FR4 | SEQ ID NO: 23 | SEQ ID NO: 29 |
| M | Ecu | Ecu* | FR1 | SEQ ID NO: 20 | SEQ ID NO: 17 |
| | Ecu | Ecu | FR2 | SEQ ID NO: 21 | SEQ ID NO: 27 |
| | I.23 | Ecu | FR3 | SEQ ID NO: 26 | SEQ ID NO: 28 |
| | Ecu** | H20C3 | FR4 | SEQ ID NO: 23 | SEQ ID NO: 32 |
| N | Ecu | Ecu* | FR1 | SEQ ID NO: 20 | SEQ ID NO: 17 |
| | Ecu | H20C3 | FR2 | SEQ ID NO: 21 | SEQ ID NO: 30 |
| | I.23 | Ecu | FR3 | SEQ ID NO: 26 | SEQ ID NO: 28 |
| | Ecu** | H20C3 | FR4 | SEQ ID NO: 23 | SEQ ID NO: 32 |
| O | Ecu | Ecu* | FR1 | SEQ ID NO: 20 | SEQ ID NO: 17 |
| | Ecu | H20C3 | FR2 | SEQ ID NO: 21 | SEQ ID NO: 30 |
| | I.23 | H20C3 | FR3 | SEQ ID NO: 26 | SEQ ID NO: 31 |
| | Ecu** | Ecu | FR4 | SEQ ID NO: 23 | SEQ ID NO: 29 |
| P | Ecu | Ecu* | FR1 | SEQ ID NO: 20 | SEQ ID NO: 17 |
| | Ecu | Ecu | FR2 | SEQ ID NO: 21 | SEQ ID NO: 27 |
| | I.23 | H20C3 | FR3 | SEQ ID NO: 26 | SEQ ID NO: 31 |
| | Ecu** | H20C3 | FR4 | SEQ ID NO: 23 | SEQ ID NO: 32 |
| Q | I.23 | Ecu* | FR1 | SEQ ID NO: 24 | SEQ ID NO: 17 |
| | Ecu | Ecu | FR2 | SEQ ID NO: 21 | SEQ ID NO: 27 |
| | Ecu | Ecu | FR3 | SEQ ID NO: 22 | SEQ ID NO: 28 |
| | Ecu** | Ecu | FR4 | SEQ ID NO: 23 | SEQ ID NO: 29 |
| R | I.23 | Ecu* | FR1 | SEQ ID NO: 24 | SEQ ID NO: 17 |
| | Ecu | H20C3 | FR2 | SEQ ID NO: 21 | SEQ ID NO: 30 |
| | Ecu | H20C3 | FR3 | SEQ ID NO: 22 | SEQ ID NO: 31 |
| | Ecu** | H20C3 | FR4 | SEQ ID NO: 23 | SEQ ID NO: 32 |
| S | I.23 | Ecu* | FR1 | SEQ ID NO: 24 | SEQ ID NO: 17 |
| | Ecu | H20C3 | FR2 | SEQ ID NO: 21 | SEQ ID NO: 30 |
| | Ecu | Ecu | FR3 | SEQ ID NO: 22 | SEQ ID NO: 28 |
| | Ecu** | Ecu | FR4 | SEQ ID NO: 23 | SEQ ID NO: 29 |
| T | I.23 | Ecu* | FR1 | SEQ ID NO: 24 | SEQ ID NO: 17 |
| | Ecu | Ecu | FR2 | SEQ ID NO: 21 | SEQ ID NO: 27 |
| | Ecu | H20C3 | FR3 | SEQ ID NO: 22 | SEQ ID NO: 31 |
| | Ecu** | Ecu | FR4 | SEQ ID NO: 23 | SEQ ID NO: 29 |
| U | I.23 | Ecu* | FR1 | SEQ ID NO: 24 | SEQ ID NO: 17 |
| | Ecu | Ecu | FR2 | SEQ ID NO: 21 | SEQ ID NO: 27 |
| | Ecu | Ecu | FR3 | SEQ ID NO: 22 | SEQ ID NO: 28 |
| | Ecu** | H20C3 | FR4 | SEQ ID NO: 23 | SEQ ID NO: 32 |
| V | I.23 | Ecu* | FR1 | SEQ ID NO: 24 | SEQ ID NO: 17 |
| | Ecu | H20C3 | FR2 | SEQ ID NO: 21 | SEQ ID NO: 30 |
| | Ecu | Ecu | FR3 | SEQ ID NO: 22 | SEQ ID NO: 28 |
| | Ecu** | H20C3 | FR4 | SEQ ID NO: 23 | SEQ ID NO: 32 |
| W | I.23 | Ecu* | FR1 | SEQ ID NO: 24 | SEQ ID NO: 17 |
| | Ecu | H20C3 | FR2 | SEQ ID NO: 21 | SEQ ID NO: 30 |
| | Ecu | H20C3 | FR3 | SEQ ID NO: 22 | SEQ ID NO: 31 |
| | Ecu** | Ecu | FR4 | SEQ ID NO: 23 | SEQ ID NO: 29 |
| X | I.23 | Ecu* | FR1 | SEQ ID NO: 24 | SEQ ID NO: 17 |
| | Ecu | Ecu | FR2 | SEQ ID NO: 21 | SEQ ID NO: 27 |
| | Ecu | H20C3 | FR3 | SEQ ID NO: 22 | SEQ ID NO: 31 |
| | Ecu** | H20C3 | FR4 | SEQ ID NO: 23 | SEQ ID NO: 32 |
| Y | I.23 | Ecu* | FR1 | SEQ ID NO: 24 | SEQ ID NO: 17 |
| | Ecu | Ecu | FR2 | SEQ ID NO: 21 | SEQ ID NO: 27 |
| | I.23 | Ecu | FR3 | SEQ ID NO: 26 | SEQ ID NO: 28 |
| | Ecu** | Ecu | FR4 | SEQ ID NO: 23 | SEQ ID NO: 29 |
| Z | I.23 | Ecu* | FR1 | SEQ ID NO: 24 | SEQ ID NO: 17 |
| | Ecu | H20C3 | FR2 | SEQ ID NO: 21 | SEQ ID NO: 30 |
| | I.23 | H20C3 | FR3 | SEQ ID NO: 26 | SEQ ID NO: 31 |
| | Ecu** | H20C3 | FR4 | SEQ ID NO: 23 | SEQ ID NO: 32 |
| AA | I.23 | Ecu* | FR1 | SEQ ID NO: 24 | SEQ ID NO: 17 |
| | Ecu | H20C3 | FR2 | SEQ ID NO: 21 | SEQ ID NO: 30 |
| | I.23 | Ecu | FR3 | SEQ ID NO: 26 | SEQ ID NO: 28 |
| | Ecu** | Ecu | FR4 | SEQ ID NO: 23 | SEQ ID NO: 29 |
| BB | I.23 | Ecu* | FR1 | SEQ ID NO: 24 | SEQ ID NO: 17 |
| | Ecu | Ecu | FR2 | SEQ ID NO: 21 | SEQ ID NO: 27 |
| | I.23 | H20C3 | FR3 | SEQ ID NO: 26 | SEQ ID NO: 31 |
| | Ecu** | Ecu | FR4 | SEQ ID NO: 23 | SEQ ID NO: 29 |
| CC | I.23 | Ecu* | FR1 | SEQ ID NO: 24 | SEQ ID NO: 17 |
| | Ecu | Ecu | FR2 | SEQ ID NO: 21 | SEQ ID NO: 27 |
| | I.23 | Ecu | FR3 | SEQ ID NO: 26 | SEQ ID NO: 28 |
| | Ecu** | H20C3 | FR4 | SEQ ID NO: 23 | SEQ ID NO: 32 |
| DD | I.23 | Ecu* | FR1 | SEQ ID NO: 24 | SEQ ID NO: 17 |
| | Ecu | H20C3 | FR2 | SEQ ID NO: 21 | SEQ ID NO: 30 |
| | I.23 | Ecu | FR3 | SEQ ID NO: 26 | SEQ ID NO: 28 |
| | Ecu** | H20C3 | FR4 | SEQ ID NO: 23 | SEQ ID NO: 32 |
| EE | I.23 | Ecu* | FR1 | SEQ ID NO: 24 | SEQ ID NO: 17 |
| | Ecu | H20C3 | FR2 | SEQ ID NO: 21 | SEQ ID NO: 30 |
| | I.23 | H20C3 | FR3 | SEQ ID NO: 26 | SEQ ID NO: 31 |
| | Ecu** | Ecu | FR4 | SEQ ID NO: 23 | SEQ ID NO: 29 |
| FF | I.23 | Ecu* | FR1 | SEQ ID NO: 24 | SEQ ID NO: 17 |
| | Ecu | Ecu | FR2 | SEQ ID NO: 21 | SEQ ID NO: 27 |
| | I.23 | H20C3 | FR3 | SEQ ID NO: 26 | SEQ ID NO: 31 |
| | Ecu** | H20C3 | FR4 | SEQ ID NO: 23 | SEQ ID NO: 32 |
| GG | Ecu | Ecu* | FR1 | SEQ ID NO: 20 | SEQ ID NO: 17 |
| | I.23 | Ecu | FR2 | SEQ ID NO: 25 | SEQ ID NO: 27 |
| | I.23 | Ecu | FR3 | SEQ ID NO: 26 | SEQ ID NO: 28 |
| | Ecu** | Ecu | FR4 | SEQ ID NO: 23 | SEQ ID NO: 29 |
| HH | Ecu | Ecu* | FR1 | SEQ ID NO: 20 | SEQ ID NO: 17 |
| | I.23 | H20C3 | FR2 | SEQ ID NO: 25 | SEQ ID NO: 30 |
| | I.23 | H20C3 | FR3 | SEQ ID NO: 26 | SEQ ID NO: 31 |
| | Ecu** | H20C3 | FR4 | SEQ ID NO: 23 | SEQ ID NO: 32 |
| II | Ecu | Ecu* | FR1 | SEQ ID NO: 20 | SEQ ID NO: 17 |
| | I.23 | H20C3 | FR2 | SEQ ID NO: 25 | SEQ ID NO: 30 |
| | I.23 | Ecu | FR3 | SEQ ID NO: 26 | SEQ ID NO: 28 |
| | Ecu** | Ecu | FR4 | SEQ ID NO: 23 | SEQ ID NO: 29 |
| JJ | Ecu | Ecu* | FR1 | SEQ ID NO: 20 | SEQ ID NO: 17 |
| | I.23 | Ecu | FR2 | SEQ ID NO: 25 | SEQ ID NO: 27 |
| | I.23 | H20C3 | FR3 | SEQ ID NO: 26 | SEQ ID NO: 31 |
| | Ecu** | Ecu | FR4 | SEQ ID NO: 23 | SEQ ID NO: 29 |
| KK | Ecu | Ecu* | FR1 | SEQ ID NO: 20 | SEQ ID NO: 17 |
| | I.23 | Ecu | FR2 | SEQ ID NO: 25 | SEQ ID NO: 27 |
| | I.23 | Ecu | FR3 | SEQ ID NO: 26 | SEQ ID NO: 28 |
| | Ecu** | H20C3 | FR4 | SEQ ID NO: 23 | SEQ ID NO: 32 |
| LL | Ecu | Ecu* | FR1 | SEQ ID NO: 20 | SEQ ID NO: 17 |
| | I.23 | H20C3 | FR2 | SEQ ID NO: 25 | SEQ ID NO: 30 |
| | I.23 | Ecu | FR3 | SEQ ID NO: 26 | SEQ ID NO: 28 |
| | Ecu** | H20C3 | FR4 | SEQ ID NO: 23 | SEQ ID NO: 32 |
| MM | Ecu | Ecu* | FR1 | SEQ ID NO: 20 | SEQ ID NO: 17 |
| | I.23 | H20C3 | FR2 | SEQ ID NO: 25 | SEQ ID NO: 30 |
| | I.23 | H20C3 | FR3 | SEQ ID NO: 26 | SEQ ID NO: 31 |
| | Ecu** | Ecu | FR4 | SEQ ID NO: 23 | SEQ ID NO: 29 |
| NN | Ecu | Ecu* | FR1 | SEQ ID NO: 20 | SEQ ID NO: 17 |
| | I.23 | Ecu | FR2 | SEQ ID NO: 25 | SEQ ID NO: 27 |
| | I.23 | H20C3 | FR3 | SEQ ID NO: 26 | SEQ ID NO: 31 |
| | Ecu** | H20C3 | FR4 | SEQ ID NO: 23 | SEQ ID NO: 32 |
| OO | I.23 | Ecu* | FR1 | SEQ ID NO: 24 | SEQ ID NO: 17 |
| | I.23 | Ecu | FR2 | SEQ ID NO: 25 | SEQ ID NO: 27 |
| | Ecu | Ecu | FR3 | SEQ ID NO: 22 | SEQ ID NO: 28 |
| | Ecu** | Ecu | FR4 | SEQ ID NO: 23 | SEQ ID NO: 29 |
| PP | I.23 | Ecu* | FR1 | SEQ ID NO: 24 | SEQ ID NO: 17 |
| | I.23 | H20C3 | FR2 | SEQ ID NO: 25 | SEQ ID NO: 30 |
| | Ecu | H20C3 | FR3 | SEQ ID NO: 22 | SEQ ID NO: 31 |
| | Ecu** | H20C3 | FR4 | SEQ ID NO: 23 | SEQ ID NO: 32 |
| QQ | I.23 | Ecu* | FR1 | SEQ ID NO: 24 | SEQ ID NO: 17 |
| | I.23 | H20C3 | FR2 | SEQ ID NO: 25 | SEQ ID NO: 30 |
| | Ecu | Ecu | FR3 | SEQ ID NO: 22 | SEQ ID NO: 28 |
| | Ecu** | Ecu | FR4 | SEQ ID NO: 23 | SEQ ID NO: 29 |
| RR | I.23 | Ecu* | FR1 | SEQ ID NO: 24 | SEQ ID NO: 17 |
| | I.23 | Ecu | FR2 | SEQ ID NO: 25 | SEQ ID NO: 27 |
| | Ecu | H20C3 | FR3 | SEQ ID NO: 22 | SEQ ID NO: 31 |
| | Ecu** | Ecu | FR4 | SEQ ID NO: 23 | SEQ ID NO: 29 |
| SS | I.23 | Ecu* | FR1 | SEQ ID NO: 24 | SEQ ID NO: 17 |
| | I.23 | Ecu | FR2 | SEQ ID NO: 25 | SEQ ID NO: 27 |
| | Ecu | Ecu | FR3 | SEQ ID NO: 22 | SEQ ID NO: 28 |
| | Ecu** | H20C3 | FR4 | SEQ ID NO: 23 | SEQ ID NO: 32 |
| TT | I.23 | Ecu* | FR1 | SEQ ID NO: 24 | SEQ ID NO: 17 |
| | I.23 | H20C3 | FR2 | SEQ ID NO: 25 | SEQ ID NO: 30 |
| | Ecu | Ecu | FR3 | SEQ ID NO: 22 | SEQ ID NO: 28 |
| | Ecu** | H20C3 | FR4 | SEQ ID NO: 23 | SEQ ID NO: 32 |
| UU | I.23 | Ecu* | FR1 | SEQ ID NO: 24 | SEQ ID NO: 17 |
| | I.23 | H20C3 | FR2 | SEQ ID NO: 25 | SEQ ID NO: 30 |
| | Ecu | H20C3 | FR3 | SEQ ID NO: 22 | SEQ ID NO: 31 |
| | Ecu** | Ecu | FR4 | SEQ ID NO: 23 | SEQ ID NO: 29 |
| VV | I.23 | Ecu* | FR1 | SEQ ID NO: 24 | SEQ ID NO: 17 |
| | I.23 | Ecu | FR2 | SEQ ID NO: 25 | SEQ ID NO: 27 |
| | Ecu | H20C3 | FR3 | SEQ ID NO: 22 | SEQ ID NO: 31 |
| | Ecu** | H20C3 | FR4 | SEQ ID NO: 23 | SEQ ID NO: 32 |
| WW | Ecu | Ecu* | FR1 | SEQ ID NO: 20 | SEQ ID NO: 17 |
| | Ecu | H20C3 | FR2 | SEQ ID NO: 21 | SEQ ID NO: 30 |
| | Ecu | Ecu | FR3 | SEQ ID NO: 22 | SEQ ID NO: 28 |
| | Ecu** | Ecu | FR4 | SEQ ID NO: 23 | SEQ ID NO: 29 |
| XX | Ecu | Ecu* | FR1 | SEQ ID NO: 20 | SEQ ID NO: 17 |
| | Ecu | Ecu | FR2 | SEQ ID NO: 21 | SEQ ID NO: 27 |
| | Ecu | H20C3 | FR3 | SEQ ID NO: 22 | SEQ ID NO: 31 |
| | Ecu** | Ecu | FR4 | SEQ ID NO: 23 | SEQ ID NO: 29 |
| YY | Ecu | Ecu* | FR1 | SEQ ID NO: 20 | SEQ ID NO: 17 |
| | Ecu | Ecu | FR2 | SEQ ID NO: 21 | SEQ ID NO: 27 |
| | Ecu | Ecu | FR3 | SEQ ID NO: 22 | SEQ ID NO: 28 |
| | Ecu** | H20C3 | FR4 | SEQ ID NO: 23 | SEQ ID NO: 32 |
| ZZ | Ecu | Ecu* | FR1 | SEQ ID NO: 20 | SEQ ID NO: 17 |
| | Ecu | H20C3 | FR2 | SEQ ID NO: 21 | SEQ ID NO: 30 |
| | Ecu | Ecu | FR3 | SEQ ID NO: 22 | SEQ ID NO: 28 |
| | Ecu** | H20C3 | FR4 | SEQ ID NO: 23 | SEQ ID NO: 32 |
| AAA | Ecu | Ecu* | FR1 | SEQ ID NO: 20 | SEQ ID NO: 17 |
| | Ecu | H20C3 | FR2 | SEQ ID NO: 21 | SEQ ID NO: 30 |
| | Ecu | H20C3 | FR3 | SEQ ID NO: 22 | SEQ ID NO: 31 |
| | Ecu** | Ecu | FR4 | SEQ ID NO: 23 | SEQ ID NO: 29 |
| BBB | Ecu | Ecu* | FR1 | SEQ ID NO: 20 | SEQ ID NO: 17 |
| | Ecu | Ecu | FR2 | SEQ ID NO: 21 | SEQ ID NO: 27 |
| | Ecu | H20C3 | FR3 | SEQ ID NO: 22 | SEQ ID NO: 31 |
| | Ecu** | H20C3 | FR4 | SEQ ID NO: 23 | SEQ ID NO: 32 |
| CCC | I.23 | Ecu* | FR1 | SEQ ID NO: 24 | SEQ ID NO: 17 |
| | I.23 | H20C3 | FR2 | SEQ ID NO: 25 | SEQ ID NO: 30 |
| | I.23 | Ecu | FR3 | SEQ ID NO: 26 | SEQ ID NO: 28 |
| | Ecu** | Ecu | FR4 | SEQ ID NO: 23 | SEQ ID NO: 29 |
| DDD | I.23 | Ecu* | FR1 | SEQ ID NO: 24 | SEQ ID NO: 17 |
| | I.23 | Ecu | FR2 | SEQ ID NO: 25 | SEQ ID NO: 27 |
| | I.23 | H20C3 | FR3 | SEQ ID NO: 26 | SEQ ID NO: 31 |
| | Ecu** | Ecu | FR4 | SEQ ID NO: 23 | SEQ ID NO: 29 |
| EEE | I.23 | Ecu* | FR1 | SEQ ID NO: 24 | SEQ ID NO: 17 |
| | I.23 | Ecu | FR2 | SEQ ID NO: 25 | SEQ ID NO: 27 |
| | I.23 | Ecu | FR3 | SEQ ID NO: 26 | SEQ ID NO: 28 |
| | Ecu** | H20C3 | FR4 | SEQ ID NO: 23 | SEQ ID NO: 32 |
| FFF | I.23 | Ecu* | FR1 | SEQ ID NO: 24 | SEQ ID NO: 17 |
| | I.23 | H20C3 | FR2 | SEQ ID NO: 25 | SEQ ID NO: 30 |
| | I.23 | Ecu | FR3 | SEQ ID NO: 26 | SEQ ID NO: 28 |
| | Ecu** | H20C3 | FR4 | SEQ ID NO: 23 | SEQ ID NO: 32 |
| GGG | I.23 | Ecu* | FR1 | SEQ ID NO: 24 | SEQ ID NO: 17 |
| | I.23 | H20C3 | FR2 | SEQ ID NO: 25 | SEQ ID NO: 30 |
| | I.23 | H20C3 | FR3 | SEQ ID NO: 26 | SEQ ID NO: 31 |
| | Ecu** | Ecu | FR4 | SEQ ID NO: 23 | SEQ ID NO: 29 |
| HHH | I.23 | Ecu* | FR1 | SEQ ID NO: 24 | SEQ ID NO: 17 |
| | I.23 | Ecu | FR2 | SEQ ID NO: 25 | SEQ ID NO: 27 |
| | I.23 | H20C3 | FR3 | SEQ ID NO: 26 | SEQ ID NO: 31 |
| | Ecu** | H20C3 | FR4 | SEQ ID NO: 23 | SEQ ID NO: 32 |

Ecu* refers to the FR1 amino acid sequence of the eculizumab heavy chain polypeptide as defined under Chothia or the FR1 amino acid sequence of the H20C3 heavy chain polypeptide as defined under Chothia, which FR1 regions are identical to each other.
Ecu** refers to the FR4 amino acid sequence of the eculizumab light chain polypeptide as defined under Chothia or the FR4 amino acid sequence of the I.23 light chain polypeptide as defined under Chothia, which FR4 regions are identical to each other.

In some embodiments, one or more (e.g., one, two, three, four, five, six, seven, or all eight) of the above framework regions of an engineered antibody can be altered so as to comprise one or more (e.g., two, three, four, five, six, seven, eight, nine, or 10 or more) amino acid substitutions. An engineered antibody that comprises these one or more substitutions is sometimes referred to as a “variant engineered antibody.” Such substitutions may be introduced if, e.g., the engineered antibody binds to the antigen recognized by a donor antibody with a lower affinity as compared to the affinity of the donor antibody for the antigen. In some embodiments, one or more of the framework regions of a variant engineered antibody comprise fewer than 10 (e.g., fewer than nine, eight, seven, six, five, four, three, two, or one) substitutions. In some embodiments, only one framework region comprises an amino acid substitution. In some embodiments, more than one framework region comprises an amino acid substitution. All that is required is that the resulting engineered antibody, when administered to a human, is less immunogenic than the corresponding donor antibody in a human. In some embodiments, the above framework region amino acid sequences are not substituted.

The amino acid substitutions can be conservative substitutions or non-conservative substitutions. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

The amino acid sequences of the light and heavy chain polypeptides can include one or more (e.g., one, two, three, four, five, six, seven, eight, nine, or 10 or more) amino acids inserted as “spacers” between the various segments (e.g., between a donor CDR and an acceptor framework region of an engineered antibody). Insertion of the spacer sequences can be useful, e.g., to recover antigen-binding affinity that may have been lost during the CDR grafting process (see below). See, e.g., Maynard and Georgiou (2001) Ann Rev Biomed Engineering 2:339-376. For example, a spacer amino acid sequence can be inserted between LFR1 and LCDR1. In some embodiments, a spacer amino acid sequence is inserted between LCDR1 and LFR2. In some embodiments, a spacer amino acid sequence is inserted between LFR2 and LCDR2. In some embodiments, a spacer amino acid sequence is inserted between LCDR2 and LFR3. In some embodiments, a spacer amino acid sequence is inserted between LFR3 and LCDR3. In some embodiments, a spacer amino acid sequence is inserted between LCDR3 and LFR4. In some embodiments, a spacer amino acid sequence is inserted between HFR1 and HCDR1. In some embodiments, a spacer amino acid sequence is inserted between HCDR1 and HFR2. In some embodiments, a spacer amino acid sequence is inserted between HFR2 and HCDR2. In some embodiments, a spacer amino acid sequence is inserted between HCDR2 and HFR3. In some embodiments, a spacer amino acid sequence is inserted between HFR3 and HCDR3. In some embodiments, a spacer amino acid sequence is inserted between HCDR3 and HFR4. In some embodiments, spacer sequences are inserted between all of the segments of the light chain polypeptide and/or all of the segments of the heavy chain polypeptide. In some embodiments, no spacers are introduced between any of the component elements of a light chain or heavy chain variable region. All that is required of an engineered antibody that comprises one or more spacer sequences is that the antibody: (a) retains the ability to bind to the same antigen as the donor antibody and (b) is less immunogenic in a human as compared to the immunogenicity of the donor antibody in a human.

As used herein, the term “antibody” refers to a whole or intact antibody molecule (e.g., IgM, IgG (including IgG1, IgG2, IgG3, and IgG4), IgA, IgD, or IgE) or any antigen-binding fragment thereof. The term antibody includes, e.g., a chimerized or chimeric antibody, a humanized antibody, a deimmunized antibody, and a fully human antibody. Antigen-binding fragments of an antibody include, e.g., a single chain antibody, a single chain Fv fragment (scFv), an Fd fragment, an Fab fragment, an Fab′ fragment, or an F(ab′)₂ fragment. An scFv fragment is a single polypeptide chain that includes both the heavy and light chain variable regions of the antibody from which the scFv is derived. In addition, intrabodies, minibodies, triabodies, and diabodies (see, e.g., Todorovska et al. (2001) J Immunol Methods 248(1):47-66; Hudson and Kortt (1999) J Immunol Methods 231(1):177-189; Poljak (1994) Structure 2(12):1121-1123; Rondon and Marasco (1997) Annual Review of Microbiology 51:257-283, the disclosures of each of which are incorporated herein by reference in their entirety) are also included in the definition of antibody and are compatible for use in the methods described herein. Bispecific antibodies are also embraced by the term “antibody.” Bispecific antibodies are monoclonal, preferably human or humanized, antibodies that have binding specificities for at least two different antigens.

The disclosure also embraces variant forms of bispecific antibodies such as the tetravalent dual variable domain immunoglobulin (DVD-Ig) molecules described in Wu et al. (2007) Nat Biotechnol 25(11):1290-1297. The DVD-Ig molecules are designed such that two different light chain variable domains (VL) from two different parent antibodies are linked in tandem directly or via a short linker by recombinant DNA techniques, followed by the light chain constant domain. Methods for generating DVD-Ig molecules from two parent antibodies are further described in, e.g., PCT Publication Nos. WO 08/024,188 and WO 07/024,715, the disclosures of each of which are incorporated herein by reference in their entirety.

As used herein, a “donor antibody” is an antibody for which a practitioner wishes to obtain, using the methods described herein, a variant of the antibody (an engineered antibody) that: (i) binds to the same antigen as the donor antibody; and (ii) has one or more (e.g., one, two, three, four, five, six, or seven or more) improved characteristics as compared to the donor antibody—particularly a decreased level of immunogenicity in a human as compared to the immunogenicity of the donor antibody. The donor antibody can be made in or derived from any of a variety of species, e.g., mammals such as non-human primates (e.g., monkeys, baboons, macaques, lemurs, apes, orangutans, gorillas, or chimpanzees), horses, cattle, pigs, sheep, goats, dogs, cats, rabbits, guinea pigs, gerbils, hamsters, rats, and mice. In some instances, the donor antibody can be a humanized or fully human antibody that when administered to a human, elicits a neutralizing HAHA response in the human. The humanized antibody can be an altered antibody that comprises one or more non-human germline framework regions. A fully human antibody can be one that comprises one or more non-germline human framework regions. For example, the human donor antibody can comprise one or more framework regions that were subject to somatic hypermutation and thus is no longer germline per se. (See, e.g., Abbas, Lichtman, and Pober (2000) “Cellular and Molecular Immunology,” 4^{th} Edition, W.B. Saunders Company (ISBN: 0721682332)). In some embodiments, the donor antibody is not a humanized or fully human antibody.

An engineered antibody can be derived from any donor antibody that specifically binds to an antigen and the binding of such donor antibody to its antigen results or is expected to result in a therapeutic effect in a human. For example, the donor antibody can bind to a microbial pathogen (e.g., virus, bacterium, protozoon, or parasite) protein such as, e.g., tetanus toxin; diphtheria toxin; or any of a variety of viral surface proteins (e.g., cytomegalovirus (CMV) glycoproteins B, H and gCIII; human immunodeficiency virus 1 (HIV-I) envelope glycoproteins; Rous sarcoma virus (RSV) envelope glycoproteins; herpes simplex virus (HSV) envelope glycoproteins; Epstein Barr virus (EBV) envelope glycoproteins; varicella-zoster virus (VZV) envelope glycoproteins; human papilloma virus (HPV) envelope glycoproteins; influenza virus glycoproteins; and Hepatitis virus family surface antigens). An engineered antibody produced from such a donor antibody is expected to be useful to treat microbial infections in a human. In some embodiments, the antibody can bind to an infectious protein such as, but not limited to, Protease Resistant Protein (PrP^{Sc}). In some embodiments, the donor antibody can bind to a growth factor, a cytokine, or a chemokine Growth factors can include, e.g., vascular endothelial growth factor (VEGF), insulin-like growth factor (IGF), bone morphogenic protein (BMP), granulocyte-colony stimulating factor (G-CSF), granulocyte-macrophage colony stimulating factor (GM-CSF), nerve growth factor (NGF); a neurotrophin, platelet-derived growth factor (PDGF), erythropoietin (EPO), thrombopoietin (TPO), myostatin (GDF-8), growth differentiation factor-9 (GDF9), basic fibroblast growth factor (bFGF or FGF2), epidermal growth factor (EGF), hepatocyte growth factor (HGF), and a neuregulin (e.g., NRG1, NRG2, NRG3, or NRG4). Cytokines include, e.g., interferons (e.g., IFNγ), tumor necrosis factor (e.g., TNFα or TNFβ), and the interleukins (e.g., IL-1 to IL-33 (e.g., IL-1, IL-2, IL-3, IL-4, IL-5, IL-6, IL-7, IL-8, IL-9, IL-10, IL-12, IL-13, or IL-15)). Chemokines include, e.g., I-309, TCA-3, MCP-I, MIP-1α, MIP-1β, RANTES, C10, MRP-2, MARC, MCP-3, MCP-2, MRP-2, CCF18, Eotaxin, MCP-5, MCP-4, NCC-I, HCC-I, leukotactin-1, LEC, NCC-4, CCL21, TARC, PARC, or Eotaxin-2. In some embodiments, the donor antibody can bind to a human complement protein such as, e.g., C1, C1q, C1r, C1s, C4, C4a, C4b, C3, C3a, C3b, C2, C2a, C2b, C5, C5a, C5b, C6, C7, C8, C9, properdin, complement factor B, complement factor D, MBL, MASP1, MASP2, or MASP3. In some embodiments, the donor antibody binds to an Fc portion of an antibody such as, e.g., the Fc portion of IgM, IgG (including IgG1, IgG2, IgG3, and IgG4), IgA, IgD, or IgE. A donor antibody can bind to a cell surface protein. Cell surface proteins include, e.g., a G protein coupled receptor (GPCR), a chemokine receptor, a cytokine receptor, or a receptor tyrosine kinase (RTK). The chemokine receptor can be, e.g., CCR1, CCR2, CCR3, CCR4, CCR5, CCR6, CCR7, CCR8, CXCR1, CXCR2, CXCR3, CXCR4, or CCX-CKR2. The cytokine receptors include, e.g., IL-1R, IL-2R, IL-3R, IL-4R, IL-5R, IL-6R, IL-8R, TNFβR1, TNFβR2, c-kit receptor, interferon (IFNα or IFNβ) receptor, IFN gamma receptor, granulocyte macrophage colony stimulating factor (GM-CSF) receptor, granulocyte colony stimulating factor (G-CSF) receptor, and prolactin receptor. RTKs include, e.g., EGF receptor, insulin receptor, PDGF receptor, FGF receptor, VEGF receptor, and HGF receptor. In some embodiments, the donor antibody binds to HER2/neu/ErbB2, HER3, or HER4.

In some embodiments, the donor antibody binds to a cancer antigen (e.g., a mutant form of a cancer antigen) such as, but not limited to, MART-1/Melan-A, gp100, adenosine deaminase-binding protein (ADAbp), FAP, cyclophilin b, colorectal associated antigen (CRC) C017-1A/GA733, carcinoembryonic antigen (CEA), CAP-I, CAP-2, etv6, AMLI, prostate specific antigen (PSA), PSA-1, PSA-2, PSA-3, prostate-specific membrane antigen (PSMA), T-cell receptor/CD3-zeta chain, CD20, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE B3), MAGE-Xp4 (MAGE-B4), MAGE-C1, MAGE-C2, MAGE-C3, MAGE-C4, MAGE-05, GAGE-1, GAGE-2, GAGE-3, GAGE-4, GAGE-5, GAGE-6, GAGE-7, GAGE-8, and GAGE-9.

In some embodiments, the donor antibody can bind to a human protein selected from the group consisting of: ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; ANGPT1; ANGPT2; ANGPTL3; ANGPTL4; ANPEP; APC; APOC1; AR; AZGP1 (zinc-α-glycoprotein); B7.1; B7.2; BAD; BAFF; BAG1; BAI1; BCL2; BCL6; BDNF; BLNK; BLR1 (MDR15); BIyS; bone morphogenic protein (BMP)1; BMP2; BMP3B (GDF10); BMP4; BMP6; BMP8; BMPR1A; BMPR1B; BMPR2; BPAG1 (plectin); BRCA1; BRCA2; C19orf10 (IL27w); complement component C3; complement component C3a; complement component C3b; complement component C4a; complement component C4b; complement component C5; complement component C5a; complement component C5b; complement component C6; complement component C7; complement component C8; complement component C9; complement factor D; complement factor B; C5aR1; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MEP-2); SLC; exodus-2; CCL22 (MDC/STC-I); CCL23 (MPIF-1); CCL24 (MPIF-2/eotaxin-2); CCL25 (TECK); CCL26 (eotaxin-3); CCL27 (CTACK/ILC); CCL28; CCL3 (MIP-1α); CCL4 (MIP-1b); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB); CCR3 (CKR3/CMKBR3); CCR4; CCR5 (CMKBR5/ChemR13); CCR6 (CMKBR6/CKR-L3/STRL22/DRY6); CCR7 (CKR7/EB11); CCR8 (CMKBR8/TER1/CKR-L1); CCR9 (GPR-9-6); CCRL1 (VSHK1); CCRL2 (L-CCR); CD164; CD19; CD1C; CD20; CD200 (OX-2); CD200R; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21Wap1/Cip1); CDKN1B (p27Kip1); CDKN1C; CDKN2A (p16INK4a); CDKN2B; CDKN2C; CDKN3; CEBPB; CERI; CHGA; CHGB; chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CLU (clusterin); CMKLR1; CMKOR1 (RDC1); CNR1; COL18A1; COL1A1; COL4A3; COL6A1; CR2; CRP; CSF1 (M-CSF); CSF2 (GM-CSF); CSF3 (GCSF); CTLA4; CTNNB1 (β-catenin); CTSB (cathepsin B); CX3CL1 (SCYD1); CX3CR1 (V28); CXCL1 (GRO1); CXCL10; CXCL11 (I-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; DAB21P; DES; DKFZp451J0118; DNCL1; DPP4; DR6; E2F1; ECGF1; EDG1; EFNA1; EFNA3; EFNB2; EGF; EGFR; ELAC2; endocan; ENG; ENO1; ENO2; ENO3; EPHB4; EPG; EPO; ERBB2 (Her-2); EREG; ERK8; ESR1; ESR2; F3 (TF); FADD; FasL; FASN; fFCERIA; FCER2; FCGR3A; FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FLJ12584; FLJ25530; FLRT1 (fibronectin); FLT1; FOS; FOSL1 (FRA-I); FY (DARC); GABRP (GABAa); GAGEB1; GAGEC1; GALNAC4S-6ST; GATA3; GDF5; GFI1; GGT1; GM-CSF; GNAS1; GNRH1; GPR2 (CCR10); GPR31; GPR44; GPR81 (FKSG80); GRCC10 (C10); GRP; GSN (Gelsolin); GSTP1; HAVCR1; HAVCR2; HDAC4; HDAC5; HDAC7A; HDAC9; HGF; HIF1A; HIP1; histamine and histamine receptors; HLA-A; HLA-DRA; HM74; HMOX1; HUMCYT2A; ICEBERG; ICOSL; ID2; IFN-α; IFNA1; IFNA2; IFNA4; IFNA5; IFNA6; IFNA7; IFNB1; IFNγ; IFNW1; IGBP1; IGF1; IGF1R; IGF2; IGFBP2; IGFBP3; IGFBP6; IL-1; IL-10; IL-10RA; IL-10RB; IL11; IL11RA; IL-12; IL12A; IL12B; IL12RB1; IL12RB2; DL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1; IL1RL2; IL1RN; IL2; IL20; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; IL7R; IL8; IL8RA; IL8RB; IL8RB; IL9; IL9R; ILK; INHA; INHBA; INSL3; INSL4; IRAKI; IRAK2; ITGA1; ITGA2; ITGA3; ITGA6 (α6 integrin); ITGAV; ITGB3; ITGB4 (β4 integrin); JAGI; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); LAMAS; LEP (leptin); Lingo-p75; Lingo-Troy; LPS; LTA (TNF-β); LTB; LTB4R (GPR16); LTB4R2; LTBR; MACMARCKS; MAG or Omgp; MAP2K7 (c-Jun); MDK; MIB1; midkine; MIF; MIP-2; MKI67 (Ki-67); MMP2; MMP9; MS4A1; MSMB; MT3 (metallothionectin-III); MTSS1; MUC1 (mucin); MYC; MYD88; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NR0B1; NR0B2; NR1D1; NR1D2; NR1H2; NR1H3; NR1H4; NR1I2; NR1I3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PART1; PATE; PAWR; PCA3; PCNA; PDGFA; PDGFB; PECAM1; PF4 (CXCL4); PGF; PGR; phosphacan; PIAS2; PIK3CG; PLAU (uPA); PLG; PLXDC1; PPBP (CXCL7); PPID; PR1; PRKCQ; PRKD1; PRL; PROC; PROK2; properdin; PSAP; PSCA; PTAFR; PTEN; PTGS2 (COX-2); PTN; RAC2 (P21Rac2); RARB; RGS1; RGS13; RGS3; RNF110 (ZNF144); ROBO2; S100A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINA3; SERPINB5 (maspin); SERPINE1 (PAI-1); SERPINF1; SHBG; SfcAZ; SLA2; SLC2A2; SLC33A1; SLC43A1; SLIT2; SPP1; SPRR1B (Spr1); ST6GAL1; STAB1; STAT6; STEAP; STEAP2; TB4R2; TBX21; TCP10; TDGF1; TEK; TGFA; TGFB1; TGFB1I1; TGFB2; TGFB3; TGFBI; TGFBR1; TGFBR2; TGFBR3; TH1L; THBS1 (thrombospondin-1); THBS2; THBS4; THPO; TIE (Tie-1); TIMP3; tissue factor; TLR10; TLR2; TLR3; TLR4; TLR5; TLR6; TLR7; TLR8; TLR9; TNF; TNF-α; TNFAIP2 (B94); TNFAIP3; TNFRSF11A; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; a Toll-like receptor; TOP2A (topoisomerase IIa); p53; TPM1; TPM2; TRADD; TRAF1; TRAF2; TRAF3; TRAF4; TRAF5; TRAF6; TREM1; TREM2; TRPC6; TSLP; TWEAK; VEGF; VEGFB; VEGFC; versican; VHL C5; VLA-4; XCL1 (lymphotactin); XCL2; XCR1 (GPR5/CCXCR1); YY1; and ZFPM2.

Suitable donor antibodies also include various therapeutic antibodies that are approved for use, in clinical trials, or in development for clinical use. Such antibodies include, e.g., rituximab (Rituxan®, IDEC/Genentech/Roche), a chimeric anti-CD20 antibody approved to treat Non-Hodgkin's lymphoma; HuMax-CD20, an anti-CD20 currently being developed by Genmab; AME-133 (Applied Molecular Evolution); hA20 (Immunomedics, Inc.); HumaLYM (Intracel); PRO70769 (International patent application no. PCT/US2003/040426); trastuzumab (Herceptin®, Genentech), a humanized anti-Her2/neu antibody approved to treat breast cancer; pertuzumab (rhuMab-2C4, Omnitarg®), currently being developed by Genentech; cetuximab (Erbitux®, Imclone); ABX-EGF currently being developed by Abgenix-Immunex-Amgen; HuMax-EGFr, currently being developed by Genmab; 425, EMD55900, EMD62000, and EMD72000 (Merck KGaA) (see U.S. Pat. No. 5,558,864; Murthy et al. (1987) Arch Biochem Biophys 252(2):549-60; Rodecket al. (1987) J Cell Biochem 35(4):315-20; and Kettleborough et al. (1991) Protein Eng 4(7):773-83); ICR62 (Institute of Cancer Research) (International publication no. WO 95/20045; Modjtahedi et al. (1993) J Cell Biophys 22(1-3):129-46; Modjtahedi et al. (1993) Br J Cancer 67(2):247-53; Modjtahedi et al. (1996) Br J Cancer 73(2):228-35; Modjtahedi et al. (2003) Int J Cancer 105(2):273-80); TheraCIM hR3 (YM Biosciences, Canada and Centro de Immunologia Molecular, Cuba (U.S. Pat. No. 5,891,996; U.S. Pat. No. 6,506,883; Mateo et al. (1997) Immunotechnology 3(1):71-81)); mAb-806 (Ludwig Institute for Cancer Research, Memorial Sloan-Kettering) (Jungbluth et al. (2003) Proc Natl Acad Sci USA 100(2):639-44); KSB-102 (KS Biomedix); MR1-I (IVAX, National Cancer Institute) (PCT WO 0162931A2); alemtuzumab (Campath®, Millenium), a humanized monoclonal antibody currently approved for treatment of B-cell chronic lymphocytic leukemia; muromonab-CD3 (Orthoclone OKT3®), an anti-CD3 antibody developed by Ortho Biotech/Johnson & Johnson; ibritumomab tiuxetan (Zevalin®), an anti-CD20 antibody developed by IDEC/Schering AG; gemtuzumab ozogamicin (Mylotarg®), an anti-CD33 (p67 protein) antibody developed by Celltech/Wyeth; alefacept (Amevive®), an anti-LFA-3 Fc fusion developed by Biogen; abciximab (ReoPro®), developed by Centocor/Lilly; basiliximab (Simulect®), developed by Novartis; palivizumab (Synagis®), developed by Medimmune; infliximab (Remicade®), an anti-TNFα antibody developed by Centocor; adalimumab (Humira®), an anti-TNFα antibody developed by Abbott; Humicade®, an anti-TNFα antibody developed by Celltech; golimumab (CNTO-148), a fully human anti-TNF antibody developed by Centocor; an anti-CD147 antibody being developed by Abgenix; ABX-IL8, an anti-IL8 antibody being developed by Abgenix; ABX-MA1, an anti-MUC18 antibody being developed by Abgenix; pemtumomab (R1549, ⁹⁰Y-muHMFG1), an anti-MUC1 in development by Antisoma; Therex (R155O), an anti-MUC1 antibody being developed by Antisoma; AngioMab (AS 1405), being developed by Antisoma; HuBC-I, being developed by Antisoma; Thioplatin (AS 1407) being developed by Antisoma; Antegren® (natalizumab) being developed by Biogen Idec and Elan; CAT-152, an anti-TGF-β2 antibody being developed by Cambridge Antibody Technology; ABT 874 (J695), an anti-IL-12 p40 antibody being developed by Abbott; CAT-192, an anti-TGFβ1 antibody being developed by Cambridge Antibody Technology and Genzyme; CAT-213, an anti-Eotaxin1 antibody being developed by Cambridge Antibody Technology; LymphoStat-B®, an anti-Blys antibody being developed by Cambridge Antibody Technology and Human Genome Sciences Inc.; TRAIL-R1 mAb, an anti-TRAIL-R1 antibody being developed by Cambridge Antibody Technology and Human Genome Sciences, Inc.; Avastin® (bevacizumab, rhuMAb-VEGF), an anti-VEGF antibody being developed by Genentech; Xolair® (Omalizumab), an anti-IgE antibody being developed by Genentech; Raptiva® (Efalizumab), an anti-CD11a antibody being developed by Genentech and Xoma; MLN-02 Antibody (formerly LDP-02), being developed by Genentech and Millennium Pharmaceuticals; HuMax CD4, an anti-CD4 antibody being developed by Genmab; HuMax-EL15, an anti-IL-15 antibody being developed by Genmab and Amgen; HuMax-Inflam, being developed by Genmab and Medarex, HuMax-Cancer; HuMax-Lymphoma, being developed by Genmab and Amgen; HuMax-TAC, being developed by Genmab; DDEC-131, an anti-CD40L antibody being developed by IDEC Pharmaceuticals; IDEC-151 (Clenoliximab), an anti-CD4 antibody being developed by IDEC Pharmaceuticals; BDEC-114, an anti-CD80 antibody being developed by IDEC Pharmaceuticals; IDEC-152, an anti-CD23 being developed by IDEC Pharmaceuticals; BEC2, an anti-idiotypic antibody being developed by Imclone; IMC-1C11, an anti-KDR antibody being developed by Imclone; DC101, an anti-flk-1 antibody being developed by Imclone; anti-VE cadherin antibodies being developed by Imclone; CEA-Cide® (labetuzumab), an anti-carcinoembryonic antigen (CEA) antibody being developed by Immunomedics; LymphoCide® (Epratuzumab), an anti-CD22 antibody being developed by Immunomedics; AFP-Cide, being developed by Immunomedics; MyelomaCide, being developed by Immunomedics; LkoCide, being developed by Immunomedics; ProstaCide, being developed by Immunomedics; MDX-010, an anti-CTLA4 antibody being developed by Medarex; MDX-060, an anti-CD30 antibody being developed by Medarex; MDX-070 being developed by Medarex; MDX-018 being developed by Medarex; Osidem® (IDM-I), an anti-Her2 antibody being developed by Medarex and Immuno-Designed Molecules; HuMax®-CD4, an anti-CD4 antibody being developed by Medarex and Genmab; HuMax-IL15, an anti-EL15 antibody being developed by Medarex and Genmab; CNTO 148, an anti-TNFα antibody being developed by Medarex and Centocor/Johnson & Johnson; CNTO 1275, an anti-cytokine antibody being developed by Centocor/Johnson & Johnson; MOR101 and MOR102, anti-intercellular adhesion molecule-1 (ICAM-1) (CD54) antibodies being developed by MorphoSys; MOR201, an anti-fibroblast growth factor receptor 3 (FGFR-3) antibody being developed by MorphoSys; Nuvion® (visilizumab), an anti-CD3 antibody being developed by Protein Design Labs; HuZAF®, an anti-gamma interferon antibody being developed by Protein Design Labs; Anti-α5β1 Integrin antibody, being developed by Protein Design Labs; ING-I, an anti-EpCAM antibody being developed by Xoma; Xolair® (Omalizumab) a humanized anti-IgE antibody developed by Genentech and Novartis; and MLNO1, an anti-β2 integrin antibody being developed by Xoma.

It is understood that the form of the donor antibody and its corresponding engineered antibody can be the same or different. For example, in some embodiments, the donor antibody and its corresponding engineered antibody are whole antibodies. In some embodiments, the donor antibody is an antibody fragment (e.g., a Fab or a scFv fragment of an antibody) and its corresponding engineered antibody is also an antibody fragment (e.g., an Fab or an scFv fragment of an antibody). However, in some embodiments, the donor antibody is a whole antibody and its corresponding engineered antibody is a fragment of an antibody or vice versa.

Methods for generating an engineered antibody are described below.

### Methods for Generating an Engineered Antibody

Methods for generating an engineered antibody require CDR amino acid sequences of a donor antibody and at least the variable region framework regions of an acceptor antibody. As described above, optionally, the engineered antibody can comprise one or more constant regions (e.g., the constant region of the acceptor antibody such as the Fc region of the heavy chain amino acid sequence depicted in SEQ ID NO:6). The acceptor antibody can comprise a light chain variable domain having the following amino acid sequence segments in order: LFR1-LCDR1-LFR2-LCDR2-LFR3-LCDR3-LFR4. LFR1, LFR2, LFR3, and LFR4 can be the framework regions obtained from a light chain variable domain having the amino acid sequence depicted in SEQ ID NO:2 or SEQ ID NO:8. Exemplary amino acid sequences for light chain framework regions as well as exemplary sets of the framework regions are described herein. (See, e.g., Tables 1 and 3-5.)

The acceptor antibody heavy chain variable domain can have the following amino acid sequence segments in order: HFR1-HCDR1-HFR2-HCDR2-HFR3-HCDR3-HFR4. HFR1, HFR2, HFR3, and HFR4 can be framework regions obtained from a heavy chain variable region polypeptide having the amino acid sequence depicted in SEQ ID NO:5 or SEQ ID NO:7. Exemplary amino acid sequences for heavy chain framework regions as well as exemplary sets of the framework regions are described herein. (See, e.g., Tables 2-5.)

The methods include replacing CDRs of the acceptor antibody (e.g., LCDR1, LCDR2, LCDR3, HCDR1, HCDR2, and HCDR3) with a set of CDRs from the donor antibody. One of skill in the art of antibody engineering would readily be able to determine the position and amino acid sequence of the CDR and framework regions in each of the donor and acceptor antibodies. As described above, CDR and framework regions of antibodies can be delineated by reference to, e.g., Kabat et al. (1991), supra, Chothia et al. (1989), supra, or a combined Kabat-Chothia definition. Identification of CDR and framework regions of antibodies under Kabat, Chothia, and combined Kabat-Chothia definitions is also exemplified in FIGS. 1 and 2 and in Thomas et al. (1996, supra).

Methods for grafting CDR sequences from a donor antibody to the framework regions of an acceptor antibody are well known in the art and are described in, e.g., Jones et al. (1986) Nature 321:522-525; Verhoeyen et al. (1988) Science 239(4847):1534-1536; Riechmann et al. (1988) Nature 332:323-327; Queen et al. (1989) Proc Natl Acad Sci USA 86:10029-10033; PCT publication no. WO 93/011237; Kettleborough et al. (1991) Protein Engineering, Design and Selection 4:773-783; Benny K. C. Lo (2004) “Antibody Engineering: Methods and Protocols,” Humana Press (ISBN: 1588290921); Borrebaek (1992) “Antibody Engineering, A Practical Guide,” W.H. Freeman and Co., NY; and Borrebaek (1995) “Antibody Engineering,” 2^{nd} Edition, Oxford University Press, NY, Oxford. For example, CDRs from a donor antibody can be grafted onto framework regions of an acceptor antibody using overlap extension polymerase chain reaction (PCR) techniques as described in, e.g., Daugherty et al. (1991) Nucleic Acids Res 19(9):2471-2476; Roguska et al. (1996) Protein Engineering 9(10):895-904; and Yazaki et al. (2004) Protein Engineering, Design & Selection 17(5):481-489. Suitable methods for grafting a set of donor CDRs to an acceptor antibody are also described in Thomas et al. (1996), supra.

In some embodiments, where the selected CDR amino acid sequences are short sequences (e.g., fewer than 10-15 amino acids in length), nucleic acids encoding the CDRs can be chemically synthesized as described in, e.g., Shiraishi et al. (2007) Nucleic Acids Symposium Series 51(1):129-130 and U.S. Pat. No. 6,995,259. For a given nucleic acid sequence encoding an acceptor antibody, the region of the nucleic acid sequence encoding the CDRs can be replaced with the chemically synthesized nucleic acids using standard molecular biology techniques. The 5′ and 3′ ends of the chemically synthesized nucleic acids can be synthesized to comprise sticky end restriction enzyme sites for use in cloning the nucleic acids into the nucleic acid encoding the variable region of the donor antibody. Methods for expressing and purifying an engineered antibody are known in the art and described herein.

Following the CDR grafting and expression of the engineered antibody (see below), the engineered antibody can be assayed for its ability to bind to the same antigen as the donor antibody. Suitable methods for determining whether an antibody binds to a protein are known in the art. For example, the binding of an antibody to a protein antigen can be detected and/or quantified using a variety of techniques such as, but not limited to, Western blot, dot blot, surface plasmon resonance (SPR) method (e.g., BIAcore system; Pharmacia Biosensor AB, Uppsala, Sweden and Piscataway, N.J.), Octet, or enzyme-linked immunosorbent assay (ELISA).

In some embodiments, the binding affinity between an engineered antibody and its cognate antigen can be determined. Methods for determining the affinity of an engineered antibody for a protein antigen are known in the art. For example, the binding of an antibody to a protein antigen can be quantified using a variety of techniques such as, but not limited to, Western blot, dot blot, SPR, Octet, or ELISA techniques. See, e.g., Harlow and Lane (1988), supra; Benny K. C. Lo (2004), supra; Borrebaek (1992), supra; Johne et al. (1993) J Immunol Meth 160:191-198; Jonsson et al. (1993) Ann Biol Clin 51:19-26; and Jonsson et al. (1991) Biotechniques 11:620-627.

Preferably, the engineered antibodies will specifically bind to the same antigen as the donor antibody. The binding of an antibody to an antigen is considered specific when the association constant (Kₐ) is higher than 10⁶ M⁻¹. Thus, an antibody can specifically bind to a protein with a Kₐ of at least (or greater than) 10⁶ (e.g., at least or greater than 10⁷, 10⁸, 10⁹, 10¹⁰, 10¹¹, 10¹², 10¹³, 10¹⁴, or 10¹⁵ or higher) M⁻¹.

CDR grafting can often be performed such that an engineered antibody will have approximately the same affinity for an antigen as compared to the affinity of the donor antibody for the same antigen. See, e.g., Jones et al. (1986), supra; Verhoeyen et al. (1988), supra; and Yazaki et al. (2004), supra. In some embodiments, the engineered antibody has an improved affinity for an antigen as compared to the affinity of the donor antibody for the antigen.

In some embodiments, an engineered antibody may have a lower affinity for an antigen as compared to the affinity of the donor antibody for the same antigen. In such cases, the lost affinity can be partially or fully recovered using, e.g., affinity maturation of the CDR sequences as described in, e.g., Gram et al. (1992) Proc Natl Acad Sci USA 89(8):3576-3580; U.S. Pat. No. 7,432,063; and PCT Publication Nos. WO 02/036738 and WO 04/055182.

The lost affinity may also be partially or fully recovered (or even sometimes exceeded) using antibody reshaping techniques as described in, e.g., Kettleborough et al. (1991) Protein Engineering 4(7):773-783; Tempest et al. (1991) BioTechnol 9:266-271; Hale et al. (1988) Lancet 2:1394-1399; and Gorman et al. (1991) Proc Natl Acad Sci USA 88:4181-4185. Computational methods for antibody reshaping have been described in, e.g., Padlan (1991) Mol Immunol 28:489-498. One heavy chain variable framework residue—position 71 (as defined by Kabat et al.)—has been identified as important for antigen binding. See, e.g., Tramontano et al. (1990) J Mol Biol 215:175-182. Using structural data from a series of immunoglobulin molecules, the authors observed that the conformation of CDR2 was dependent, in part, on its interaction with residue 71. Retention of residue 71 was shown to be important for obtaining acceptable affinity in a reshaped anti-EGF receptor antibody (Kettleborough et al. (1991), supra and Krauss et al. (2004) Br J Cancer 90:1863-1870). Heavy chain variable region framework residues 48, 66, and 67 (as defined by Kabat et al.) have also been shown to be important for retention of antibody affinity during CDR grafting and reshaping. (Id.) Moreover, Riechmann et al. (1988; supra) discloses the contribution of heavy chain variable region framework residues 27 and 30 (as defined by Kabat et al.) for restoring the affinity of a CDR-grafted anti-CAMPATH-1 antibody. Saldanha et al. ((1999) Mol Immunol 36(11-12):709-719) demonstrated that a backmutation introduced at position 9 of the human kappa IV light chain FR1 restored binding affinity of a previously unsuccessfully humanized antibody and found that the mutation also increased the secretion levels of the antibody in COS cells. Thomas et al. (1996), supra discusses the importance of V_{H} framework region position 78 for maintaining the function of human antibodies. See also, e.g., Foote and Winter (1992) J Mol Biol 224:487-499. As described in the working examples and in Thomas et al. (1996), supra, V_{H} positions 28 and 30 can also be important for antibody stability and function. Accordingly, it is understood that any of the foregoing modifications can be made to, or can be present in, the engineered antibodies described herein, so long as the engineered antibody remains less immunogenic in a human as compared to the immunogenicity of the original donor antibody in a human.

Suitable methods for recovering antigen-binding affinity that was lost during the reshaping of an antibody are also described in, e.g., U.S. Pat. Nos. 6,180,370; 6,350,861; and 5,693,762, the disclosures of each of which are incorporated by reference in their entirety. For example, U.S. Pat. No. 6,180,370 (issued to Queen et al.) describes methods for restoring affinity of an engineered antibody by replacing at least one (e.g., one, two, three, four, five, or six or more) amino acid of an engineered antibody variable region (e.g., an engineered antibody framework region) with the corresponding amino acid present in the donor antibody variable region (a so-called “back mutation”). The methods include, e.g., comparing (aligning) a framework region of the engineered antibody with the corresponding framework region in the donor antibody and identifying amino acids that are: (a) rare for that position, (b) immediately adjacent to a CDR, and/or (c) amino acid(s) that are predicted to be within about 3 Å of a CDR in a three-dimensional space. The identified amino acids can be particularly amenable to back mutations to restore lost affinity to the engineered antibody. One or more (e.g., one, two, three, four, five, or six or more) back mutations can be introduced to a single framework region of the engineered antibody or to more than one (e.g., two, three, four, five, six, seven, or eight) framework region of the engineered antibody. In some embodiments, back mutations can be introduced in a sufficient number to render an engineered antibody framework region greater than 65 (e.g., 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95 or more) % identical to the corresponding donor antibody framework region. In some embodiments, one or more back-mutations can be introduced in a sufficient number to render an engineered antibody variable region (e.g., the light chain variable region or the heavy chain variable region) greater than 65 (e.g., 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95 or more) % identical to the corresponding variable region of the donor antibody. All that is required of the engineered antibody containing the back mutation(s) is that the engineered antibody is less immunogenic in a human as compared to the immunogenicity of the donor antibody in a human.

As discussed above, the reshaping or affinity maturation techniques can be used to introduce one or more (e.g., two, three, four, five, six, seven, eight, nine, or 10 or more) amino acid substitutions (e.g., conservative or non-conservative substitutions) into one or more (e.g., one, two, three, four, five, six, seven, or all eight) of the engineered antibody framework regions (e.g., HFR1, HFR2, HFR3, HFR4, LFR1, LFR2, LFR3, or LFR4). In some embodiments, the framework regions in total comprise fewer than 10 (e.g., fewer than nine, eight, seven, six, five, four, three, two, or one) substitutions. In some embodiments, only one framework region is altered during the reshaping process (e.g., to introduce one or more amino acid substitutions into the region). In some embodiments, more than one (e.g., two, three, four, five, or all six) framework region(s) is/are altered to comprise one or more amino acid substitutions. All that is required is that the resulting engineered antibody when administered to a human is less immunogenic than the corresponding donor antibody in a human. In some embodiments, amino acid substitutions are performed prior to the grafting process. In some embodiments, amino acid substitutions are performed after the grafting process.

As discussed above, one of ordinary skill in the art would recognize that the exact boundaries of variable domain CDRs and framework regions can vary depending on how they are defined. For example, under the Chothia definition or the combined Kabat-Chothia definition, V_{H} positions 28 and 30 fall within the heavy chain CDR1 region. Thus, in some embodiments, one or more amino acid substitutions can be introduced into the CDRs of the engineered antibody V_{H} region and/or V_{L} region, but not into the antibody's framework regions. Such substitutions can affect antibody reshaping or affinity maturation. Accordingly, in some embodiments, antibody reshaping or maturation techniques can include introducing one or more (e.g., two, three, four, five, six, seven, eight, nine, or 10 or more) amino acid substitutions (e.g., conservative or non-conservative substitutions) into one or more (e.g., one, two, three, four, five, or all six) of the engineered antibody CDR regions (e.g., HCDR1, HCDR2, HCDR3, LCDR1, LCDR2, or LCDR3), e.g., as defined by Kabat, Chothia, or the combined Kabat-Chothia definition. In some embodiments, the CDRs in total comprise fewer than 10 (e.g., fewer than nine, eight, seven, six, five, four, three, two, or one) substitutions. In some embodiments, none of the donor CDRs is subjected to amino acid substitution prior to, or after, grafting the CDRs to the acceptor scaffold. All that is required of said substitutions is that: (a) the resulting engineered antibody when administered to a human is less immunogenic than the corresponding donor antibody in a human; and (b) the substitutions improve the affinity of the engineered antibody for a target antigen as compared to the affinity of the engineered antibody for the antigen prior to the substitutions.

In some embodiments, an engineered light chain polypeptide and an engineered heavy chain polypeptide can be generated using the methods described herein. In some embodiments, a practitioner can select to generate only an engineered light chain polypeptide or an engineered heavy chain polypeptide and use, e.g., guided selection to identify the complementary polypeptide chain (light or heavy chain polypeptide) to thereby create an engineered antibody having reduced immunogenicity in a human. For example, a practitioner who has generated an engineered light chain polypeptide can employ guided selection techniques to identify a cognate human heavy chain polypeptide sequence to thereby generate an engineered antibody that is less immunogenic in a human as compared to the donor antibody. Guided selection techniques are described in detail in, e.g., U.S. Pat. No. 5,565,332 (issued to Hoogenboom et al.), Guo-Qiang and Xian-L1 (2009) Methods Mol Biol 562:133-142, Klimka et al. (2000) Br J Cancer 83(2):252-260, and Beiboer et al. (2000) J Mol Biol 296(3):833-849. Briefly, guided selection involves pairing an antibody light chain polypeptide or an antibody heavy chain polypeptide of interest with a repertoire of human complementary (light or heavy) variable domains. The hybrid pairings are interrogated, e.g., using phage display techniques. Specific hybrid pairings that retain affinity for the antigen of interest can be selected.

In some embodiments, the humanization methods described herein can include interrogating libraries of diverse human variable regions or parts of human variable regions as described in, e.g.: U.S. Pat. No. 7,087,409; Rader et al. (1998) Proc Natl Acad Sci USA 95:8910-8915; and Steinberger et al. (2000) J Biol Chem 275(46):36073-36078. For example, a practitioner may generate an intermediate engineered light chain polypeptide cassette comprising FR3 and FR4 of the eculizumab light chain variable region and a CDR3 of a donor antibody. (It is understood that the starting cassette can be any combination of contiguous framework regions and CDR sequences, e.g.: FR1-CDR1, FR1-CDR1-FR2, FR1-CDR1-FR2-CDR2, FR1-CDR1-FR2-CDR2-FR3, CDR1-FR2-CDR2-FR3-CDR3-FR4, FR2-CDR2-FR3-CDR3-FR4, CDR2-FR3-CDR3-FR4, FR3-CDR3-FR4, CDR3-FR4, FR2-CDR2-FR3, CDR1-FR2-CDR2, etc.) The practitioner may then generate a diverse library of intermediate engineered light chain polypeptides in which the aforementioned FR3-CDR3-FR4 cassette is joined to a library of human FR1-CDR1-FR2-CDR2 cassettes. The library of intermediate light chain polypeptides can be paired with an engineered antibody heavy chain polypeptide, e.g., an engineered antibody containing at least one of the framework regions described herein and one or more CDRs from a donor antibody. The hybrid pairings can be interrogated (e.g., using phage display techniques) to identify one or more individual hybrid pairings that retain the ability to bind to the same antigen as the donor antibody and demonstrate reduced immunogenicity in a human as compared to the donor antibody. Additional methods for using exchange cassettes to humanize antibody in accordance with the methods described herein are set forth in, e.g., U.S. patent application publication nos. 20060134098 and 20050255552.

In some embodiments, PCR-directed mutagenesis can be used to introduce random mutations into the framework regions of an engineered antibody to thereby generate a library of variant engineered antibodies. In some embodiments, a library of variant engineered antibodies can be produced using PCR, wherein targeted mutations are introduced into one or more of the framework regions of an engineered antibody. At least part of the variant engineered antibody library can be screened to identify a variant engineered antibody having one or more desired characteristics such as improved affinity for an antigen and/or reduced or further reduced immunogenicity in a human as compared to the donor antibody. Methods for screening antibody libraries are well known in the art of antibody engineering and include, e.g., phage-display, bacterial display, yeast surface display, eukaryotic viral display, mammalian cell display, and cell-free (e.g., ribosomal display) antibody screening techniques (see, e.g., Etz et al. (2001) J Bacteriol 183:6924-6935; Cornelis (2000) Curr Opin Biotechnol 11:450-454; Klemm et al. (2000) Microbiology 146:3025-3032; Kieke et al. (1997) Protein Eng 10:1303-1310; Yeung et al. (2002) Biotechnol. Prog. 18:212-220; Boder et al. (2000) Methods Enzymology 328:430-444; Grabherr et al. (2001) Comb Chem High Throughput Screen 4:185-192; Michael et al. (1995) Gene Ther 2:660-668; Pereboev et al. (2001) J Virol 75:7107-7113; Schaffitzel et al. (1999) J Immunol Methods 231:119-135; and Hanes et al. (2000) Nat Biotechnol 18:1287-1292). Phage-display antibody screening involves expressing an antibody protein displayed on the phage (e.g., M13 filamentous phage or λ, T4, or T7 phage) surface. See, e.g., Sidhu (2001) Biomol Eng 18:57-63; Maruyama et al. (1994) Proc Natl Acad Sci USA 91:8273-8277; Ren and Black (1998) Gene 215:439-444; Rosenberg et al. (1996) InNovations 6:1-6; and Castagnoli et al. (2001) Comb Chem High Throughput Screen 4:121-133. Briefly, a plurality of phagemid vectors, each encoding a fusion protein of a bacteriophage coat protein (e.g., pIII or pVIII of M13 phage) and a different engineered antibody are produced using standard molecular biology techniques and then introduced into a population of bacteria (e.g., E. coli). Expression of the bacteriophage in bacteria can, in some embodiments, require use of a helper phage. In some embodiments, no helper phage are required (see, e.g., Chasteen et al. (2006) Nucleic Acids Res 34(21):e145). Phage produced from the bacteria are recovered and then contacted to, e.g., a target antigen bound to a solid support. The unbound phage are removed by washing the solid support. Following the wash step, bound phage are then eluted from the solid support, e.g., using a free target antigen competitor. Generally, any eluted phage can be considered to comprise an antibody (or fragment thereof) that binds to the target antigen. Individual phage of the population can be isolated by, e.g., infecting bacteria grown in wells of a multi-well assay plate at a multiplicity of infection of one phage per well.

To enrich the phage population for phage particles that comprise antibodies having a higher affinity for the target antigen (while reducing the proportion of phage that may bind to the antigen non-specifically), the eluted phage (described above) can be used to re-infect a population of bacterial host cells. The expressed phage are then obtained from the bacteria and again contacted to a target antigen bound to a solid support (e.g., the surface of a bead or a column). The unbound phage are removed by washing the solid support. Following the wash step, bound phage are then eluted from the solid support, e.g., using a free target antigen competitor. The number of infection-binding-elution cycles that the phage particles are subjected to generally correlates with level of enrichment for phage comprising antibodies having higher affinity for the target antigen.

Methods for antibody phage display are also described in, e.g., O'Brien and Aitken (2002) “Antibody Phage Display: Methods and Protocols,” Humana Press (ISBN 0896037118); Barbas et al. (2004) “Phage Display: A Laboratory Manual,” Cold Spring Harbor Laboratory Press (ISBN: 0879697407); and Figini et al. (1998) Cancer Res 58:991-996, the disclosure of each of which is incorporated herein by reference in its entirety. Methods for automating various steps of the antibody phage display for use in high-throughput screening campaigns are described in, e.g., Konthur and Walter (2002) TARGETS 1(1):30-36.

Additional screening methods are available for identifying an engineered antibody that binds to the same antigen as the donor antibody. For example, a practitioner of the methods can use any of a variety of filter screening methods, e.g., wherein secreted antibody fragments are trapped on a membrane that is then contacted with soluble target antigen. See, e.g., Skerra et al. (1991) Anal Biochem 196:151-5. In this case, bacteria harboring plasmid vectors that direct the secretion of Fab fragments into the bacterial periplasm are grown on a membrane or filter. The secreted fragments are allowed to diffuse to a second “capture” membrane coated with antibody which can bind the antibody fragments (e.g., anti-immunoglobulin antiserum) and the capture filter is probed with specific antigen. Antibody-enzyme conjugates can be used to detect antigen-binding antibody fragments on the capture membrane as a colored spot. The colonies are re-grown on the first membrane and the clone expressing the desired antibody fragment recovered.

A practitioner of the methods can also use ELISA techniques to screen for an engineered antibody that binds to the same antigen as the donor antibody. An individual engineered antibody expressed from a single clone, or pools of multiple engineered antibodies produced by multiple clones, can be assayed as described in, e.g., Watkins et al. (1997) Anal. Biochem. 253:37-45. A practitioner could also use colony lift binding assays, wherein the antibodies are allowed to diffuse directly onto an antigen-coated membrane. Such a method is described in, e.g., Giovannoni et al. (2001) Nucleic Acids Research 29(5):e27.

Methods for determining whether an engineered antibody is immunogenic in a human are well known in the art. For example, the engineered antibody can be administered to a human subject as part of a Phase 0 clinical study. See, e.g., Kinders et al. (2007) Molecular Interventions 7:325-334. The engineered antibody can be administered orally or transdermally, or injected (or infused) intravenously, subcutaneously, intramuscularly, intraperitoneally, intrarectally, intravaginally, intranasally, intragastrically, intratracheally, or intrapulmonarily. The antibody can be delivered directly to an appropriate lymphoid tissue (e.g. spleen, lymph node, or mucosal-associated lymphoid tissue (MALT)). If desired, booster immunizations may be given once or several (two, three, four, eight or twelve, for example) times at various times (e.g., spaced one week apart). Antibody (e.g., IgG, IgM, or IgA) responses specific for the engineered antibody can then be measured by testing for the presence of such antibodies systemically (e.g., in serum) or, for example, at various mucosal sites (e.g., in saliva or gastric and bronchoalveolar lavages) using in vitro assays familiar to those in the art, e.g., an ELISA. Commercial ELISA-based kits are available and include, e.g., the HAHA ELISA ELPCO™ Immunoassay (ALPCO Diagnostics, Salem, N.H.). Suitable methods (e.g., ELISA or SPR methods) for detecting the production by a subject (e.g., a human subject) of neutralizing antibodies that bind to and inhibit the activity of a therapeutic antibody are known in the art and exemplified in the working Examples. Suitable methods are also described in, e.g., Welt et al. (2003) Clin Cancer Res 9(4):1338-46; Aarden et al. (2008), supra; Szolar et al. (2006) J Pharm Biomed Anal 41(4):1347-1353; Lofgren et al. (2007) J Immunol 178(11):7467-7472; Ritter et al. (2001) Cancer Res 61:6851-6859; and Buist et al. (1995) Cancer Immunology, Immunotherapy 40(1):24-30. Alternatively, or in addition, since CD4⁺ T cell responses are generally required for antibody responses, in vitro CD4⁺ T cell responses to the engineered antibody can be measured using methods known in the art. Such methods include CD4⁺ T cell proliferation or lymphokine (e.g., interleukin-2, interleukin-4, or interferon-γ) production assays.

In some embodiments, the methods described herein can include determining whether a donor antibody (e.g., a humanized donor antibody) is likely to be, or is expected to be, immunogenic in a human. In some embodiments, the methods described herein can include determining in silico the potential immunogenicity of an engineered antibody in a human. Suitable computer-based methods/algorithms for predicting the potential immunogenicity of a given antibody or antibody variable regions are known in the art and include, without limitation, SYFPEITHI, TEPITOPE, BEPITOPE, RANKPEP (Harvard University), MMPred, PREDICT, MHCBench, and ABCpred. See Rammensee et al. (1999) Immunogenetics 50:213; Saha and Raghava (2007) Methods Mol Biol 409:387-394; El-Manzalawy et al. (2008) J Mol Recognit 21(4):243-255; Sturniolo et al. (1999) Nat Biotechnol 17:555; Bhasin and Raghava (2004) Bioinformatics 20(3):421-423; and van de Weert and Møller (2008), “Immunogenicity of Biopharmaceuticals,” Volume 8 of Biotechnology: Pharmaceutical Aspects, Springer Press (see Table 4.2 titled “Epitope prediction tools, databases and data sets”). The in silico determination can occur prior to the generation of the engineered antibody (e.g., an evaluation of one or more donor antibodies) and/or after the generation of the engineered antibody (e.g., before administering an engineered antibody to a human). In some embodiments, the in silico determination can occur after reshaping the engineered antibody (e.g., introducing one or more back-mutations into the engineered antibody). In some embodiments, the in silico methods can be employed to help guide a practitioner in determining which reshaping techniques to employ on an engineered antibody. For example, if a practitioner has a choice between two comparable reshaping techniques (e.g., a back-mutation at one of two different amino acid positions in a framework region), the practitioner may turn to the aforementioned in silico methods to determine which of the two techniques would likely result in the engineered antibody having the least potential for immunogenicity in a human.

Methods for Expressing an Engineered Antibody The nucleic acid(s) encoding an engineered antibody can be inserted into an expression vector that comprises transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system such that it can be maintained in two different organisms, for example in mammalian or insect cells for expression and in a prokaryotic host for cloning and amplification.

Several possible vector systems are available for the expression of cloned engineered antibody heavy chain and/or light chain polypeptides from nucleic acids in mammalian cells. One class of vectors relies upon the integration of the desired gene sequences into the host cell genome. Cells which have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet. 1:327). The selectable marker gene can be either linked to the DNA gene sequences to be expressed, or introduced into the same cell by co-transfection (Wigler et al. (1979) Cell 16:77). A second class of vectors utilizes DNA elements which confer autonomously replicating capabilities to an extrachromosomal plasmid. These vectors can be derived from animal viruses, such as bovine papillomavirus (Sarver et al. (1982) Proc Natl Acad Sci USA, 79:7147), polyoma virus (Deans et al. (1984) Proc Natl Acad Sci USA 81:1292), or SV40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, lipofectin, electroporation, viral infection, dextran-mediated transfection, polybrene-mediated transfection, and direct microinjection.

Appropriate host cells for the expression of engineered antibodies, include, e.g., yeast, bacteria, insect, and mammalian cells. Of particular interest are bacteria such as E. coli, fungi such as Saccharomyces cerevisiae and Pichia pastoris, insect cells such as SF9, mammalian cell lines (e.g., human cell lines), as well as primary cell lines. The type of host cell selected for expression of the antibodies will depend in part on the particular type of antibody to be expressed as well as the intended use of the expressed antibody. For example, a skilled artisan may choose a bacterial host for expressing a single chain antibody or a Fab fragment of an antibody, whereas the artisan may choose a mammalian cell host for whole antibody expression.

The engineered antibodies are produced from cells by culturing a host cell transformed with the expression vector comprising nucleic acid encoding the antibody under conditions, and for an amount of time, sufficient to allow expression of the antibodies. Such conditions for protein expression will vary with the choice of the expression vector and the host cell, and can be easily ascertained by one skilled in the art through routine experimentation. For example, engineered antibodies expressed in E. coli can be refolded from inclusion bodies (see, e.g., Hou et al. (1998) Cytokine 10:319-30). Bacterial expression systems and methods for their use are well known in the art. The choice of codons, suitable expression vectors and suitable host cells will vary depending on a number of factors, and may be easily optimized as needed. Engineered antibodies can be expressed in mammalian cells or in other expression systems including but not limited to yeast, baculovirus, and in vitro expression systems (see, e.g., Kaszubska et. al. (2000) Protein Expression and Purification 18:213-220).

In some embodiments, an engineered antibody can be expressed in, and purified from, transgenic animals (e.g., transgenic mammals). For example, an engineered antibody can be produced in transgenic non-human mammals (e.g., rodents) and isolated from milk as described in, e.g., Houdebine (2002) Curr Opin Biotechnol 13(6):625-629; van Kuik-Romeijn et al. (2000) Transgenic Res 9(2):155-159; and Pollock et al. (1999) J Immunol Methods 231(1-2):147-157.

Suitable antibody expression methods are also set forth in Thomas et al. (1996), supra.

Following expression, the engineered antibodies can be isolated. The term “isolated” or “purified” as applied to any of the polypeptides described herein (e.g., the engineered antibodies) refers to a polypeptide that has been separated or purified from components (e.g., proteins or other naturally-occurring biological or organic molecules) which naturally accompany it, e.g., other proteins, lipids, and nucleic acid in a prokaryote expressing the proteins. Typically, a polypeptide is purified when it constitutes at least 60 (e.g., at least 65, 70, 75, 80, 85, 90, 92, 95, 97, or 99) %, by weight, of the total protein in a sample.

The engineered antibodies can be isolated or purified in a variety of ways known to those skilled in the art depending on what other components are present in the sample. Standard purification methods include electrophoretic, molecular, immunological, and chromatographic techniques, including ion exchange, hydrophobic, affinity, and reverse-phase HPLC chromatography. For example, an engineered antibody can be purified using a standard anti-antibody column (e.g., a protein-A or protein-G column). Ultrafiltration and diafiltration techniques, in conjunction with protein concentration, are also useful. See, e.g., Scopes (1994) “Protein Purification, 3^{rd} edition,” Springer-Verlag, New York City, N.Y. The degree of purification necessary will vary depending on the desired use. In some instances, no purification of the expressed engineered antibodies will be necessary.

Methods for determining the yield or purity of an isolated engineered antibody are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

In some embodiments, endotoxin can be removed from the expressed engineered antibodies. Methods for removing endotoxin from a protein sample are known in the art. For example, endotoxin can be removed from a protein sample using a variety of commercially available reagents including, without limitation, the ProteoSpin™ Endotoxin Removal Kits (Norgen Biotek Corporation), Detoxi-Gel Endotoxin Removal Gel (Thermo Scientific; Pierce Protein Research Products), MiraCLEAN® Endotoxin Removal Kit (Minis), or Acrodisc™-Mustang® E membrane (Pall Corporation).

Methods for detecting and/or measuring the amount of endotoxin present in a sample (both before and after purification) are known in the art and commercial kits are available. For example, the concentration of endotoxin in a protein sample can be determined using the QCL-1000 Chromogenic kit (BioWhittaker), the limulus amebocyte lysate (LAL)-based kits such as the Pyrotell®, Pyrotell®-T, Pyrochrome®, Chromo-LAL, and CSE kits available from the Associates of Cape Cod Incorporated.

### Pharmaceutical Compositions

Compositions comprising an engineered antibody described herein can be formulated as a pharmaceutical composition. The pharmaceutical compositions will generally include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (see, e.g., Berge et al. (1977) J Pharm Sci 66:1-19).

The compositions can be formulated according to standard methods. Pharmaceutical formulation is a well-established art, and is further described in, e.g., Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7^{th} Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X). In some embodiments, a composition can be formulated, for example, as a buffered solution at a suitable concentration and suitable for storage at 2-8° C. (e.g., 4° C.). In some embodiments, a composition can be formulated for storage at a temperature below 0° C. (e.g., −20° C. or −80° C.). In some embodiments, the composition can be formulated for storage for up to 2 years (e.g., one month, two months, three months, four months, five months, six months, seven months, eight months, nine months, 10 months, 11 months, 1 year, 1½ years, or 2 years) at 2-8° C. (e.g., 4° C.). Thus, in some embodiments, the compositions described herein are stable in storage for at least 1 year at 2-8° C. (e.g., 4° C.).

The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. For example, compositions comprising an antibody or fragment intended for systemic or local delivery can be in the form of injectable or infusible solutions. Accordingly, the compositions can be formulated for administration by a parenteral mode (e.g., intravenous, subcutaneous, intraperitoneal, or intramuscular injection). “Parenteral administration,” “administered parenterally,” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intracerebral, intracranial, intracarotid and intrasternal injection and infusion (see below).

In some embodiments, an engineered antibody described herein can be formulated in a composition suitable for intrapulmonary administration to a human (e.g., for administration via nebulizer or inhaler). Methods for preparing such compositions are well known in the art and described in, e.g., U.S. patent application publication no. 20080202513; U.S. Pat. Nos. 7,112,341 and 6,019,968; and PCT application publication nos. WO 00/061178 and WO 06/122257, the disclosures of each of which are incorporated herein by reference in their entirety. Dry powder inhaler formulations and suitable systems for administration of the formulations are described in, e.g., U.S. patent application publication no. 20070235029, PCT Publication No. WO 00/69887; and U.S. Pat. No. 5,997,848.

The compositions can be formulated as a solution, microemulsion, dispersion, liposome, or other ordered structure suitable for stable storage at high concentration. Sterile injectable solutions can be prepared by incorporating an antibody (or a fragment of the antibody) described herein in the required amount in an appropriate solvent with one or a combination of ingredients enumerated above, as required, followed by filtered sterilization. Generally, dispersions are prepared by incorporating an antibody or fragment described herein into a sterile vehicle that comprises a basic dispersion medium and the required other ingredients from those enumerated above. In the case of sterile powders for the preparation of sterile injectable solutions, methods for preparation include vacuum drying and freeze-drying that yield a powder of the engineered antibody described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

In certain embodiments, the engineered antibody can be prepared with a carrier that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known in the art. See, e.g., J. R. Robinson (1978) “Sustained and Controlled Release Drug Delivery Systems,” Marcel Dekker, Inc., New York.

Nucleic acids encoding an engineered antibody can be incorporated into a gene construct to be used as a part of a gene therapy protocol to deliver nucleic acids that can be used to express and produce agents within cells (see below). Expression constructs of such components may be administered in any therapeutically effective carrier, e.g. any formulation or composition capable of effectively delivering the component gene to cells in vivo. Approaches include insertion of the subject gene in viral vectors including recombinant retroviruses, adenovirus, adeno-associated virus, lentivirus, and herpes simplex virus-1 (HSV-1), or recombinant bacterial or eukaryotic plasmids. Viral vectors can transfect cells directly; plasmid DNA can be delivered with the help of, for example, cationic liposomes (lipofectin) or derivatized (e.g., antibody conjugated), polylysine conjugates, gramicidin S, artificial viral envelopes or other such intracellular carriers, as well as direct injection of the gene construct or CaPO₄ precipitation (see, e.g., WO04/060407) carried out in vivo. (See also, “Ex vivo Approaches,” below.) Examples of suitable retroviruses include pLJ, pZIP, pWE and pEM which are known to those skilled in the art (see, e.g., Eglitis et al. (1985) Science 230:1395-1398; Danos and Mulligan (1988) Proc Natl Acad Sci USA 85:6460-6464; Wilson et al. (1988) Proc Natl Acad Sci USA 85:3014-3018; Armentano et al. (1990) Proc Natl Acad Sci USA 87:6141-6145; Huber et al. (1991) Proc Natl Acad Sci USA 88:8039-8043; Ferry et al. (1991) Proc Natl Acad Sci USA 88:8377-8381; Chowdhury et al. (1991) Science 254:1802-1805; van Beusechem et al. (1992) Proc Natl Acad Sci USA 89:7640-7644; Kay et al. (1992) Human Gene Therapy 3:641-647; Dai et al. (1992) Proc Natl Acad Sci USA 89:10892-10895; Hwu et al. (1993) J Immunol 150:4104-4115; U.S. Pat. Nos. 4,868,116 and 4,980,286; PCT Publication Nos. WO89/07136, WO89/02468, WO89/05345, and WO92/07573). Another viral gene delivery system utilizes adenovirus-derived vectors (see, e.g., Berkner et al. (1988) BioTechniques 6:616; Rosenfeld et al. (1991) Science 252:431-434; and Rosenfeld et al. (1992) Cell 68:143-155). Suitable adenoviral vectors derived from the adenovirus strain Ad type 5 d1324 or other strains of adenovirus (e.g., Ad2, Ad3, Ad7, etc.) are known to those skilled in the art. Yet another viral vector system useful for delivery of the subject gene is the adeno-associated virus (AAV). See, e.g., Flotte et al. (1992) Am J Respir Cell Mol Biol 7:349-356; Samulski et al. (1989) J. Virol. 63:3822-3828; and McLaughlin et al. (1989) J Virol 62:1963-1973.

### Applications

As described above, the engineered antibodies described herein are characterized by having, inter alia, reduced immunogenicity in a human as compared to the immunogenicity of the donor antibody from which it was derived. Accordingly, the engineered antibodies can be used in a wide variety of diagnostic and/or therapeutic applications, e.g., where the engineered antibodies are to be administered chronically to a human. While in no way intended to be limiting, several exemplary applications in which the engineered antibodies can be generated and/or used are elaborated on below.

A Therapeutic Anti-TNFα Antibody

A therapeutic, humanized anti-TNFα antibody that is administered chronically to human patients is found by a medical practitioner to elicit a human anti-human antibody (HAHA) response in a large percentage of all treated patients. Moreover, the antibodies generated in these patients substantially neutralize the therapeutic activity of the anti-TNFα antibody. Thus, it is determined that continued administration of the anti-TNFα antibody to these patients will provide little or no therapeutic benefit. The patients have a variety of severe autoimmune disorders including rheumatoid arthritis, Crohn's disease, ulcerative colitis, and ankylosing spondylitis, and they depend on the anti-TNFα antibody to effectively manage their disease.

The CDRs of the donor anti-TNFα antibody are grafted into a reduced immunogenicity antibody acceptor scaffold described herein. The newly engineered anti-TNFα antibody is tested for its ability to bind to TNFα and is found to have approximately the same affinity for TNFα as the donor anti-TNFα antibody. In a Phase 0 study, the engineered antibody is administered to a cohort of human patients once every month for six months. Blood samples are obtained from each of the patients just prior to each monthly administration and the samples are used to determine if the patients generate antibodies to the engineered antibody. It is expected that a substantially lower percentage of patients treated with the engineered antibody will develop a HAHA response as compared to the percentage of patients treated with the original humanized anti-TNFα antibody. Accordingly, it is also expected that the engineered antibody will be effective for chronic treatment of severe autoimmune disorders in a larger number of patients as compared to the original humanized anti-TNFα antibody.

A Therapeutic Anti-VEGF Antibody

A therapeutic, humanized anti-vascular endothelial growth factor (VEGF) antibody that is administered more than once to human patients is found by a medical practitioner to elicit a neutralizing HAHA response in a large percentage of treated patients. The patients have colorectal cancer and in each case, they depend on the anti-VEGF therapy to manage their cancer.

The CDRs of the donor anti-VEGF antibody are grafted into a reduced immunogenicity acceptor antibody scaffold described herein. The newly engineered anti-VEGF antibody is tested for its ability to bind to VEGF and is found to have approximately the same affinity for VEGF as the donor anti-VEGF antibody. In a Phase 0 study, the engineered antibody is administered to a cohort of human patients once every two weeks for two months. Blood samples are obtained from each of the patients just prior to each administration and the samples are used to determine if the patients generate antibodies to the engineered antibody. It is expected that a substantially lower percentage of patients treated with the engineered antibody will develop a HAHA response as compared to the percentage of patients treated with the original humanized anti-VEGF antibody. It is also expected that the engineered antibody will be effective for treatment of colorectal cancers in a larger number of patients as compared to the original humanized anti-VEGF antibody.

A Therapeutic Anti-CD20 Antibody

A therapeutic, humanized anti-CD20 antibody that is intravenously administered more than once to human patients is found by numerous medical practitioners to elicit a neutralizing HAHA response in a large percentage of treated patients. The patients have non-Hodgkin's Lymphoma and in each case, the patients depend on the anti-CD20 therapy to help treat their condition.

The CDRs of the donor anti-CD20 antibody are grafted into a reduced immunogenicity acceptor antibody scaffold described herein. The newly engineered anti-CD20 antibody is tested for its ability to bind to CD20 and is found to have a reduced affinity for CD20 as compared to the affinity of the donor anti-CD20 antibody for CD20 protein. The antibody is subjected to reshaping techniques to identify variant engineered anti-CD20 antibodies having improved affinity for CD20. Substitution mutations are introduced into two heavy chain variable region framework amino acid residues 27 and 30 (as defined by Kabat et al.; see Riechmann et al. (1988), supra). The variant engineered anti-CD20 antibody is again tested for its affinity for CD20 and is found to have an improved affinity for CD20 that is at least equivalent to the affinity of the donor anti-CD20 antibody for CD20 protein.

In a Phase 0 study, the variant engineered antibody is administered to a cohort of human patients once a week, for two months. Blood samples are obtained from each of the patients just prior to each administration and the samples are used to determine if the patients generate antibodies to the variant engineered antibody. It is expected that a substantially lower percentage of patients treated with the variant engineered antibody will develop a HAHA response as compared to the percentage of patients treated with the original humanized anti-CD20 antibody. It is also expected that the variant engineered antibody will be effective for treatment of Non-Hodgkin's Lymphoma in a larger number of patients as compared to the original humanized anti-CD20 antibody.

A Therapeutic Anti-IgE Antibody

A therapeutic, humanized anti-IgE antibody delivered more than once to human patients by way of intrapulmonary administration is found by a medical practitioner to elicit a neutralizing HAHA response in a large percentage of treated patients. The patients have asthma (moderate to high severity).

The CDRs of the donor anti-IgE antibody are grafted into a reduced immunogenicity acceptor antibody scaffold described herein. The newly engineered anti-IgE antibody is tested for its ability to bind to the IgE heavy chain constant region and is found to have approximately the same affinity for IgE as the donor anti-IgE antibody. In a Phase 0 study, the engineered antibody is administered by nebulizer to a cohort of human patients once every two weeks, for two months. Blood and sputum samples are obtained from each of the patients just prior to each administration. The samples are used to determine if the patients generate antibodies to the engineered antibody. It is expected that a substantially lower percentage of patients treated with the engineered antibody will develop a HAHA response as compared to the percentage of patients treated with the original humanized anti-IgE antibody. It is also expected that the engineered antibody will be effective for treatment of asthma in a larger number of patients as compared to the original humanized anti-IgE antibody.

The following examples are intended to illustrate, not limit, the invention.

### EXAMPLES

### Example 1

### Generation of a Humanized Acceptor Antibody Having Low Immunogenicity

A murine monoclonal antibody (“mαC5 antibody”) that specifically binds to human complement component C5 was humanized as follows to create the antibody known as eculizumab. The CDRs of the mαC5 antibody were grafted onto human framework regions having a high degree of sequence homology to the frameworks of the mαC5 antibody. The human variable regions chosen as acceptor sequences for the CDRs of the mαC5 antibody were selected by scanning the Genbank subdirectory GB-PR with the program TFASTA (NCBI) utilizing the mouse variable heavy (V_{H}) and variable light (V_{L}) sequences as the query sequences. The human V_{H} region identified from the search was the clone H20C3H (Genbank Locus No. HUMIGHRL; accession no. L02325). See, e.g., Weng et al. (1992) J Immunol 149(7):2518-2529. This human V_{H} region was derived from the human genomic V_{H} gene HG3 and the human genomic J_{H}5 gene, and contains no changes in the framework regions from these genomic genes. The human V_{L} region identified from the search was the clone I.23 (Genbank accession no. X72477). See, e.g., Klein et al. (1993) Eur J Immunol 23:3248-3271. This human V_{L} region was derived from the human genomic V_{κ} gene 012 and the genomic J_{κ}1 gene, with the introduction of an arginine (R) residue in framework region 2 (FR2) at position 38 of the mature variable region as compared to the encoded glutamine (Q) residue in the 012 genomic gene. Amino acid sequences for the H20C3 V_{H} and I.23 V_{L} sequences are set forth herein as SEQ ID NOs: 7 and 8, respectively. The CDR-framework grafting (based on Kabat-defined CDRs) was performed using overlap-extension PCR techniques. Amino acid substitutions were introduced into the H20C3 V_{H} sequence at positions 28 and 30. Specifically, the threonine at position 28 and the threonine at position 30 were substituted with an isoleucine and a serine, respectively. The isoleucine at position 28 and serine at position 30 were present in the murine anti-C5 antibody sequence from which eculizumab's CDRs were obtained. The amino acid sequences of the light chain variable region of eculizumab and of the light chain variable region of I.23 are depicted in FIG. 1. The amino acid sequences of the heavy chain variable region of eculizumab and of the heavy chain variable region of H20C3 are depicted in FIG. 2. The amino acid sequence of the complete light chain of eculizumab is depicted in SEQ ID NO:1. The amino acid sequence of the complete heavy chain of eculizumab is depicted in SEQ ID NO:4. It is noted that positions 28 and 30 fall within the Chothia CDR and that if a combined Kabat-Chothia CDR had been grafted, the same final result would have been obtained without the need for making substitutions at positions 28 and 30.

### Example 2

### Assay to Detect Human Anti-Eculizumab Antibodies

The following assay was used to detect the presence of human anti-eculizumab antibodies in biological samples from patients treated with eculizumab. The assay involves two stages: a screening stage and a confirmatory stage. The screening stage assay involved the evaluation of patient blood samples (test samples) in the context of a negative control (normal human serum; control sample) and a positive control reference standard. A patient serum or test sample was evaluated by adding 25 μL of a 2% solution of serum (v/v) from a patient treated with eculizumab to a well of a 96 well round bottom propylene assay plate. For the negative control sample, in this case, 25 μL of a 2% (v/v) normal human serum (NHS) pool was added to a well of the plate. A series of positive control standard samples were also prepared, the standards comprising different predetermined amounts (400, 100, 50, 25, 10, 5, 2, and 0 ng/mL) of an antibody that is raised against eculizumab. 25 μL of the standard samples was added to a set of wells of the plate. Each test, control, and standard sample was evaluated in triplicate.

Next, 25 μL of a solution comprising 2 μg/mL of each of: (i) eculizumab conjugated to biotin and (ii) eculizumab conjugated to ruthenium (TAG) was added to each well of the plate. Following the addition, the plate was sealed, protected from light, and incubated with shaking at room temperature for 18 hours. After the incubation, a 25 μL aliquot of a 0.5 mg/mL solution of streptavidin-coated DynaBeads (Invitrogen; Carlsbad, Calif.) was added to each well of the plate. The plate was again sealed, protected from light, and incubated with shaking at room temperature for three hours. Following the incubation, 150 mL of a buffer comprising 1% bovine serum albumin (BSA) and 0.5% Tween-20 in phosphate buffered saline was added to each well. The amount of light produced (light emission) from each well of the plate was measured using a BioVeris M-384 Detection System (Roche).

To determine whether a sample was positive and should advance to further testing in the confirmatory stage, the following screening assay was performed. The average light emission produced from the wells comprising a test sample was divided by the average light emission produced from the wells comprising the corresponding control sample. If the resulting number was less than or equal to 1.2, the test sample was considered negative. If the resulting number was greater than 1.2, the test sample was considered screening assay positive and advanced to the confirmatory assay stage.

The confirmatory assay involved a direct comparison of a post-drug test sample (blood obtained from a patient treated with eculizumab) and a corresponding blood sample from the patient prior to administration of eculizumab (hereinafter a “pre-drug sample”). The amount of eculizumab present in the post-drug test sample was determined. That determined concentration of eculizumab was then added to the predrug sample to create a “predrug+ec sample.” The addition of eculizumab to the predrug sample normalized the degree of serum matrix effect due to unlabeled drug interference. In addition, the confirmatory assay also involved an evaluation of the post-drug test sample and the predrug+ec sample in the presence of an excess amount of eculizumab as an assay signal inhibitor, which are herein referred to as “test+INHIBITOR” and “predrug+ec+INHIBITOR” samples. The addition of the excess eculizumab is to evaluate if the assay signal is drug specific.

25 μL of a test sample (2% v/v) was added to six wells of a 96 well assay plate. Similarly, 25 μL of a predrug+ec sample (2% v/v) was added to another six wells of the assay plate. To generate the test+INHIBITOR condition, 25 μL of a 50 μg/mL solution of eculizumab was added to three of the six wells comprising the test sample. Likewise, to generate the predrug+ec+INHIBITOR condition, 25 μL of a 50 μg/mL solution of eculizumab was added to three of the six wells comprising the predrug+ec sample. As described above, 25 μL of a solution containing 2 μg/mL of each of: (i) eculizumab conjugated to biotin and (ii) eculizumab-TAG, was then added to each well of the plate. Following the addition, the plate was sealed, protected from light, and incubated with shaking at room temperature for 18 hours. After the incubation, a 25 μL aliquot of a 0.5 mg/mL solution of streptavidin-coated DynaBeads was added to each well of the plate. The plate was again sealed, protected from light, and incubated with shaking for three hours at room temperature. Following the incubation, 150 μL of a buffer containing 1% bovine serum albumin (BSA) and 0.5% Tween-20 in phosphate buffered saline was added to each well. The light emission from each well of the plate was measured using a BioVeris M-384 Detection System (Roche).

To determine if a test sample is positive (that is, the sample contains a human anti-eculizumab antibody), the average light emission from each of the groups of wells was evaluated as follows. First, Ratio A was determined as the average light emission produced from the wells containing the predrug+ec sample divided by the average light emission produced from the wells containing the predrug+ec+INHIBITOR sample. Ratio A indicates the nonspecific signal changes in the background serum reduced in the presence of the inhibitor.

Next, Ratio B was calculated as the average light emission produced from wells containing the test sample divided by the average light emission produced from wells containing the test+INHIBITOR sample. Ratio B reflects any light emission changes in the test sample that are reduced in the presence of the inhibitor.

A third ratio, Ratio C, was determined as Ratio B divided by Ratio A. Ratio C thus reflects the increase, if any, in light emission resulting from the generation of a human anti-eculizumab antibody response in the patient from which the test sample was obtained. If Ratio C was less than 1.3, the test sample was considered negative in the confirmatory assay. If Ratio C was greater than 1.3, the test sample was considered positive for the potential presence of a human anti-eculizumab antibody.

### Example 3

### Assay to Detect Neutralizing Human Anti-Eculizumab Antibodies

A test sample that was considered positive in both the screening and confirmatory assays (a HAHA positive test sample) was then analyzed to determine if the human anti-eculizumab antibodies present in the test sample were capable of neutralizing eculizumab.

To prepare the samples for the assay, the amounts of complement component C5 in the predrug sample and the HAHA positive test sample were also determined. The results were used to determine the amount of C5 to add to the predrug or HAHA positive test sample so that their C5 concentrations were identical. The amount of eculizumab in the HAHA positive test sample was determined. The determined amount of eculizumab was added to the corresponding, normalized predrug sample to create the predrug+ec sample.

To prepare the assay plate, 150 μL of blocking buffer [3% BSA in phosphate buffered saline] was added to each well of a streptavidin-coated 96 well assay plate. The plate was sealed and incubated with shaking at room temperature for one hour. Following the incubation, the contents of each well were removed and the wells were washed three times with 150 μL of a wash buffer [0.05% Tween-20 in phosphate buffered saline]. After the final wash, the buffer was removed and 25 μL of a 1 μg/mL solution containing eculizumab conjugated to biotin was added to each well. The plate was sealed and incubated with shaking at 37° C. in the dark for three hours. Following the incubation, the contents of the wells were removed and the wells were washed three times with wash buffer.

After washing the wells, 25 μL of the test sample (2% v/v) was added to three wells of the plate. Similarly, 25 μL of the predrug+ec sample (2% v/v) was added to three wells of the assay plate. In addition, a series of positive control standard solutions were also prepared and 25 μL of the standards were added to a set of wells of the plate, the standards containing different predetermined amounts (50, 25, 10, 5, 2, and 0 ng/mL) of an antibody that is known to bind to and neutralize eculizumab. The plate was again sealed and incubated with shaking at 37° C. in the dark for one hour. Following the incubation, the contents of the wells were removed, and without washing, 25 μL of a 250 ng/mL solution of C5 conjugated to ruthenium was added to each well. The plate was then covered and incubated with shaking at room temperature for one hour. Following the incubation, the plate was washed three times with 150 μL of wash buffer. Next, 150 μL of 2× Read Buffer T (containing surfactant; MSD®, catalogue number R92TC-1) was added to each well. The light emission produced from each well of the assay plate was determined using an MSD® Sector Imager 2400 using MSD® Workbench Software.

To analyze the data, the following calculation was performed. The average light emission from the wells containing the predrug+ec sample was divided by the average light emission produced from wells containing the HAHA positive test sample. The resulting numerical value, if less than 1.3, was considered to indicate that the HAHA response in the test sample was non-neutralizing. A numerical value that was greater than 1.3 indicated that the HAHA positive test sample may contain neutralizing anti-eculizumab antibodies. The data for HAHA positive test samples were further analyzed to determine the extent of neutralization, or the “% suppression” of eculizumab binding activity, by the anti-eculizumab antibodies present in the patient samples. The % suppression was calculated as 100%-[(the signal obtained in the Nab assay using a sample in which no anti-eculizumab antibodies are present)/(the signal obtained in the Nab assay using a confirmatory assay positive sample containing one or more anti-eculizumab antibodies)]×100. The cut-off value, equal to or above which represents a meaningful % signal suppression in this analysis, is 23%.

### Example 4

### Low Level of Immunogenicity of Eculizumab in Human Patients

In clinical studies, eculizumab was administered intravenously to human patients at a dosage of 600 mg weekly for 4 weeks, 900 mg one week later, followed by maintenance doses of 900 mg every two weeks thereafter. Each patient received at least 68 therapeutic doses of eculizumab over two and a half years. Many of the patients received therapeutic concentrations of eculizumab over at least five years (over 130 doses). A total of 793 serum samples from 161 of the patients were tested to determine whether a human anti-human antibody (HAHA) response occurred in the patients. 49 of the serum samples were determined to be positive in the above-described screening assay. Of those 49 samples, 20 were patient samples obtained prior to administering eculizumab (pre-drug samples) and 29 samples were obtained from patients after administering eculizumab (post-drug samples). The confirmatory assay was performed only on post-drug samples. Seven (7) of the 29 post-drug samples tested positive using the above-described confirmatory assay (confirmatory assay positive samples), which suggested that anti-eculizumab antibodies might be present in the seven samples.

The confirmatory assay positive samples were subjected to the above-described Neutralizing Antibody Assay (Nab assay) in conjunction with the pre-drug samples corresponding to the confirmatory assay positive samples. As described above, the pre-drug samples were supplemented with eculizumab and complement component C5 to a concentration measured in the post-drug counterpart samples.

Only three (3) confirmatory assay positive samples were determined in the Nab assay to have a “% suppression level” value slightly higher than the cut-off value. (One of the three samples was obtained from a first patient and the other two samples were obtained from a second patient.) The “% suppression” values for the three samples were determined to be 25.7%, 27.5%, and 36.2%, respectively. Notably, the pharmacokinetic (PK) and pharmacodynamic (PD) properties of the antibody were not affected by the low level of anti-eculizumab antibodies present in the samples.

These data indicate that the eculizumab antibody, when chronically administered to human patients at therapeutic doses, is generally poorly immunogenic and, in the small minority of patients (2 out of 161 patients) in which an anti-eculizumab antibody response was detectable in the immunogenicity test, the response did not neutralize the therapeutic efficacy of the antibody.

### Example 5

### Use of a Scaffold Described Herein to Generate New Humanized Therapeutic Antibodies

The variable regions of a murine anti-human C5a antibody were subjected to humanization. The amino acid sequences of the murine light chain and heavy chain variable regions are shown below in Table 6.

TABLE 6
Amino Acid Sequences of a Murine
Anti-C5a Antibody

| SIN: | Description | Amino Acid Sequence* |
|---|---|---|
| 33 | V_{L} Amino Acid | DIVMTQSPASLAVSLGQRATISCRASESVDSYG |
| | Sequence | NSFMHWYQQKPGQPPKLLIYRASNLESGIPAR |
| | | FSGSGSRTDFTLTINPVEADDVATYYCQQSNE |
| | | DPYTFGGGTKLEIKR |
| 34 | Light Chain | RASESVDSYGNSFMH |
| | CDR1 | |
| 35 | Light Chain | RASNLES |
| | CDR2 | |
| 36 | Light Chain | QQSNEDPYT |
| | CDR3 | |
| 37 | V_{H} Amino Acid | EVQLQQSGPELVKPGSSVKISCKASGYTFTDYS |
| | Sequence | MDWVKQSHGKSLEWIGAINPNSGGTNYSQKF |
| | | KDKATLTVDKSSSTAYMELRSLTSEDSAVYYC |
| | | ASSGSYDGYYAMDYWGQGTSVTVSS |
| 38 | Heavy Chain | GYTFTDYSMD |
| | CDR1 | |
| 39 | Heavy Chain | AINPNSGGTNYSQKFKD |
| | CDR2 | |
| 40 | Heavy Chain | SGSYDGYYAMDY |
| | CDR3 | |

“SIN” in the Table refers to “SEQ ID NO.”
*CDR amino acid sequences defined according to the combined Kabat-Chothia definition (supra).

Routine molecular biological methods were employed to graft the murine antibody CDRs onto a human germline framework scaffold. Additional humanization was performed by replacing a serine residue in CDR2 of the heavy chain with an asparagine, to thereby remove a potential glycosylation site. The amino acid sequences of the humanized anti-human C5a antibody are set forth in Table 7.

TABLE 7
Amino Acid Sequences of a Humanized
Anti-C5a Antibody

| SIN: | Description | Amino Acid Sequence* |
|---|---|---|
| 41 | V_{L} Amino Acid | DIQMTQSPSSLSASVGDRVTITCRASESVDSYG |
| | Sequence | NSFMHWYQQKPGKAPKLLIYRASNLESGVPSR |
| | | FSGSGSGTDFTLTISSLQPEDFATYYCQQSNED |
| | | PYTFGGGTKVEIK |
| 9 | Light Chain | DIQMTQSPSSLSASVGDRVTITC |
| | FR1 | |
| 34 | Light Chain | RASESVDSYGNSFMH |
| | CDR1 | |
| 10 | Light Chain | WYQQKPGKAPKLLIY |
| | FR2 | |
| 35 | Light Chain | RASNLES |
| | CDR2 | |
| 11 | Light Chain | GVPSRFSGSGSGTDFTLTISSLQPEDFATYYC |
| | FR3 | |
| 36 | Light Chain | QQSNEDPYT |
| | CDR3 | |
| 42 | Light Chain | FGGGTKVEIK |
| | FR4 | |
| 43 | V_{H} Amino Acid | QVQLVQSGAEVKKPGASVKVSCKASGYTFTD |
| | Sequence | YSMDWVRQAPGQGLEWMGAINPNSGGTNYN |
| | | QKFKDRVTMTRDTSTSTVYMELSSLRSEDTAV |
| | | YYCARSGSYDGYYAMDYWGQGTTVTVSS |
| 17 | Heavy Chain | QVQLVQSGAEVKKPGASVKVSCKAS |
| | FR1 | |
| 38 | Heavy Chain | GYTFTDYSMD |
| | CDR1 | |
| 14 | Heavy Chain | WVRQAPGQGLEWMG |
| | FR2 | |
| 44 | Heavy Chain | AINPNSGGTNYNQKFKD |
| | CDR2 | |
| 15 | Heavy Chain | RVTMTRDTSTSTVYMELSSLRSEDTAVYYCAR |
| | FR3 | |
| 40 | Heavy Chain | SGSYDGYYAMDY |
| | CDR3 | |
| 45 | Heavy Chain | WGQGTTVTVSS |
| | FR4 | |

“SIN” in the Table refers to “SEQ ID NO.”
*CDR amino acid sequences defined according to the combined Kabat-Chothia definition (supra).
In bold are light chain and heavy chain framework region 4 amino acids that differ from the corresponding FR4 regions of eculizumab.

As shown in Table 7, the humanized anti-C5a antibody contains light chain framework regions 1 (SEQ ID NO:9), 2 (SEQ ID NO:10), and 3 (SEQ ID NO:11) of the eculizumab antibody and heavy chain framework regions 1 (SEQ ID NO:17), 2 (SEQ ID NO:14), and 3 (SEQ ID NO:15) of the eculizumab antibody, all of which defined under the Kabat-Chothia definition. See Tables 1 and 2 above. Light chain framework 4 (LFR4) varies from LFR4 of eculizumab by one amino acid (bolded in Table 7 above). Similarly, heavy chain framework 4 (HFR4) varies from HFR4 of eculizumab by one amino acid (also bolded in Table 7 above).

The humanized antibody was subjected to BIAcore analysis to quantify its affinity for human C5a, in part, to determine if humanization affected the binding affinity of the antibody for its antigen. See, e.g., Karlsson and Larsson (2004) Methods Mol Biol 248:389-415. Briefly, the humanized antibody was screened with 3-4 concentrations of human C5a (antigen) using a capture technique. The antibody was captured by an anti-Fc (human) antibody directly immobilized on a CM5 sensor chip with various concentrations in the range from 0.6 nM to 5.9 nM of human C5a passed over the sensor chip surface. The surface was regenerated with 20 mM HCl, 0.02% P20 after each cycle to remove bound antibody and antigen. The data were evaluated using Biacore BIAevaluation software using a 1:1 Langmuir Model Fit (Rmax:Global Fit; RI:Local Fit). Kinetics information such as kₐ (Association Rate constant), k_{d} (Dissociation Rate constant), and K_{D} (Equillibrium Dissociation constant) was obtained from the fit. The results of the analyses are as follows: kₐ≈1.93×10⁶ M⁻¹s⁻¹; k_{d}≈5.76×10⁻⁴ S⁻¹; and K_{D}≈2.98×10⁻¹⁰ M. Under similar conditions, the murine anti-C5a antibody counterpart bound to human C5a with the following parameters: kₐ≈2.76×10⁶ M⁻¹s⁻¹; k_{d}≈1.41×10⁻⁴ s⁻¹; and K_{D}≈5.12×10⁻¹⁰ M. These data indicated that humanization of the murine antibody improved the binding affinity of the antibody for human C5a (K_{D} of 5.12×10⁻¹⁰ M to 2.98×10⁻¹⁰ M). Methods for testing the humanized antibody for reduced immunogenicity in a human, as compared to the donor antibody, are known in the art and described herein.

At a minimum, these results indicate that the eculizumab framework regions described herein can be used to humanize other non-human antibodies without adversely affecting the affinity of the antibodies for their cognate antigens.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

1. A polypeptide comprising the following amino acid sequence segments in order: LFR1-L CDR 1-LFR2-LCDR2-LFR3-LCDR3-LFR4,
wherein one or more of light chain framework regions LFR1, LFR2, and LFR3 are obtained from a light chain variable region having the amino acid sequence depicted in SEQ ID NO:2 or SEQ ID NO:8, and wherein one or more of the light chain complementarity determining regions LCDR1, LCDR2, and LCDR3 are obtained from a donor antibody, with the proviso that the polypeptide does not comprise the complete amino acid sequence depicted in SEQ ID NO:2 or SEQ ID NO:8.

2. The polypeptide of claim 1, wherein LFR4 is obtained from the light chain variable region having the amino acid sequence depicted in SEQ ID NO:2 or SEQ ID NO:8.

3-4. (canceled)

5. The polypeptide of claim 1, wherein at least two of the CDRs are from the same donor antibody.

6. The polypeptide of claim 1, wherein all of the CDRs are from the same donor antibody.

7. The polypeptide of claim 1, wherein the framework regions and the CDRs are defined according to Kabat.

8. The polypeptide of claim 1, wherein the framework regions and the CDRs are defined according to Chothia.

9. (canceled)

10. The polypeptide of claim 1, wherein LFR1 comprises the amino acid sequence depicted in SEQ ID NO:9, SEQ ID NO:20, or SEQ ID NO:24.

11. The polypeptide of claim 1, wherein LFR2 comprises the amino acid sequence depicted in SEQ ID NO:10, SEQ ID NO:18, SEQ ID NO:21, or SEQ ID NO:25.

12. The polypeptide of claim 1, wherein LFR3 comprises the amino acid sequence depicted in SEQ ID NO:11, SEQ ID NO:22, or SEQ ID NO:26.

13. The polypeptide of claim 1, wherein the LFR4 comprises the amino acid sequence depicted in SEQ ID NO:12 or SEQ ID NO:23.

14. The polypeptide of claim 1, wherein:
(i) LFR1 comprises the amino acid sequence depicted in SEQ ID NO:9; LFR2 comprises the amino acid sequence depicted in SEQ ID NO:10; LFR3 comprises the amino acid sequence depicted in SEQ ID NO:11; and LFR4 comprises the amino acid sequence depicted in SEQ ID NO:12;
(ii) LFR1 comprises the amino acid sequence depicted in SEQ ID NO:9; LFR2 comprises the amino acid sequence depicted in SEQ ID NO:18; LFR3 comprises the amino acid sequence depicted in SEQ ID NO:11; and LFR4 comprises the amino acid sequence depicted in SEQ ID NO:12;
(iii) LFR1 comprises the amino acid sequence depicted in SEQ ID NO:20; LFR2 comprises the amino acid sequence depicted in SEQ ID NO:21; LFR3 comprises the amino acid sequence depicted in SEQ ID NO:22; and LFR4 comprises the amino acid sequence depicted in SEQ ID NO:23; or
(iv) LFR1 comprises the amino acid sequence depicted in SEQ ID NO:24; LFR2 comprises the amino acid sequence depicted in SEQ ID NO:25; LFR3 comprises the amino acid sequence depicted in SEQ ID NO:26; and LFR4 comprises the amino acid sequence depicted in SEQ ID NO:23.

15-23. (canceled)

24. A polypeptide comprising the following amino acid sequence segments in order: HFR1-HCDR1-HFR2-HCDR2-HFR3-HCDR3-HFR4,
wherein one or more of heavy chain framework regions HFR1, HFR2, and HFR3 are obtained from a heavy chain variable region having the amino acid sequence depicted in SEQ ID NO:5 or SEQ ID NO:7, and wherein one or more of the heavy chain complementarity determining regions HCDR1, HCDR2, and HCDR3 are obtained from a donor antibody, with the proviso that the polypeptide does not comprise the complete amino acid sequence depicted in SEQ ID NO:5 or SEQ ID NO:7.

25. The polypeptide of claim 24, wherein LFR4 is obtained from the heavy chain variable region having the amino acid sequence depicted in SEQ ID NO:5 or SEQ ID NO:7.

26-27. (canceled)

28. The polypeptide of claim 24, wherein at least two of the CDRs are from the same donor antibody.

29. The polypeptide of claim 24, wherein all of the CDRs are from the same donor antibody.

30. The polypeptide of claim 24, wherein the framework regions and the CDRs are defined according to Kabat.

31. The polypeptide of claim 24, wherein the framework regions and the CDRs are defined according to Chothia.

32. The polypeptide of claim 24, wherein the framework regions and the CDRs are defined according to a combined Kabat-Chothia definition.

33. The polypeptide of claim 24, wherein HFR1 comprises the amino acid sequence depicted in any one of SEQ ID NOs: 13, 17, or 19.

34. The polypeptide of claim 24, wherein HFR2 comprises the amino acid sequence depicted in SEQ ID NO:14, SEQ ID NO:27, or SEQ ID NO:30.

35. The polypeptide of claim 24, wherein HFR3 comprises the amino acid sequence depicted in SEQ ID NO:15, SEQ ID NO:28, or SEQ ID NO:31.

36. The polypeptide of claim 24, wherein HFR4 comprises the amino acid sequence depicted in SEQ ID NO:16, SEQ ID NO:29, or SEQ ID NO:32.

37. The polypeptide of claim 24, wherein:
(i) HFR1 comprises the amino acid sequence depicted in SEQ ID NO:13; HFR2 comprises the amino acid sequence depicted in SEQ ID NO:14; HFR3 comprises the amino acid sequence depicted in SEQ ID NO:15; and HFR4 comprises the amino acid sequence depicted in SEQ ID NO:16;
(ii) HFR1 comprises the amino acid sequence depicted in SEQ ID NO:17; HFR2 comprises the amino acid sequence depicted in SEQ ID NO:14; HFR3 comprises the amino acid sequence depicted in SEQ ID NO:15; and HFR4 comprises the amino acid sequence depicted in SEQ ID NO:16;
(iii) HFR1 comprises the amino acid sequence depicted in SEQ ID NO:19; HFR2 comprises the amino acid sequence depicted in SEQ ID NO:14; HFR3 comprises the amino acid sequence depicted in SEQ ID NO:15; and HFR4 comprises the amino acid sequence depicted in SEQ ID NO:16;
(iv) HFR1 comprises the amino acid sequence depicted in SEQ ID NO:17; HFR2 comprises the amino acid sequence depicted in SEQ ID NO:27; HFR3 comprises the amino acid sequence depicted in SEQ ID NO:28; and HFR4 comprises the amino acid sequence depicted in SEQ ID NO:29; or
(v) HFR1 comprises the amino acid sequence depicted in SEQ ID NO:17; HFR2 comprises the amino acid sequence depicted in SEQ ID NO:30; HFR3 comprises the amino acid sequence depicted in SEQ ID NO:31; and HFR4 comprises the amino acid sequence depicted in SEQ ID NO:32.

38-48. (canceled)

49. An engineered antibody comprising: (i) a light chain polypeptide and (ii) a heavy chain polypeptide, wherein the light chain polypeptide comprises the following amino acid sequence segments in order: LFR1-LCDR1-LFR2-LCDR2-LFR3-LCDR3-LFR4,
wherein light chain framework regions LFR1, LFR2, and LFR3 are obtained from a light chain variable region having the amino acid sequence depicted in SEQ ID NO:2 or SEQ ID NO:8, and wherein one or more of the light chain complementarity determining regions LCDR1, LCDR2, and LCDR3 are obtained from a donor antibody, with the proviso that the light chain polypeptide does not comprise the complete amino acid sequence depicted in SEQ ID NO:2 or SEQ ID NO:8; and
wherein the heavy chain polypeptide comprises the following amino acid sequence segments in order: HFR1-HCDR1-HFR2-HCDR2-HFR3-HCDR3-HFR4, wherein heavy chain framework regions HFR1, HFR2, and HFR3 are obtained from a heavy chain variable region having the amino acid sequence depicted in SEQ ID NO:5 or SEQ ID NO:7, and wherein one or more of the heavy chain complementarity determining regions HCDR1, HCDR2, and HCDR3 are obtained from a donor antibody, with the proviso that the heavy chain polypeptide does not comprise the complete amino acid sequence depicted in SEQ ID NO:5 or SEQ ID NO:7.

50-109. (canceled)

110. A nucleic acid encoding the polypeptide of claim 1.

111-116. (canceled)

117. A method for generating an engineered light chain antibody variable region that is less immunogenic in a human as compared to the immunogenicity of a donor light chain variable region, the method comprising:
providing information comprising: (i) an acceptor light chain antibody variable region amino acid sequence comprising the amino acid sequence depicted in SEQ ID NO:2 or SEQ ID NO:8 or (ii) a nucleic acid sequence encoding the acceptor light chain antibody variable region amino acid sequence;
providing information comprising: (iii) at least one donor antibody light chain variable region amino acid sequence or (iv) a nucleic acid sequence encoding the donor antibody light chain variable region amino acid sequence;
replacing one or more CDRs of the acceptor light chain antibody variable region with one or more CDRs from the donor antibody light chain variable region to thereby generate an engineered light chain variable region that is less immunogenic in a human as compared to the immunogenicity of the donor antibody light chain variable region, with the proviso that the engineered light chain variable region does not comprise a light chain polypeptide comprising the complete amino acid sequence depicted in SEQ ID NO:2 or SEQ ID NO:8.

118-121. (canceled)

122. A method for generating an engineered heavy chain antibody variable region that is less immunogenic in a human as compared to the immunogenicity of a donor antibody heavy chain variable region, the method comprising:
providing information comprising: (i) an acceptor heavy chain antibody variable region amino acid sequence comprising the amino acid sequence depicted in SEQ ID NO:5 or SEQ ID NO:7 or (ii) a nucleic acid sequence encoding the acceptor heavy chain antibody variable region amino acid sequence;
providing information comprising: (iii) at least one donor antibody heavy chain variable region amino acid sequence or (iv) a nucleic acid sequence encoding the donor antibody heavy chain variable region amino acid sequence;
replacing one or more CDRs of the acceptor heavy chain antibody variable region with one or more CDRs from the donor antibody heavy chain variable region to thereby generate an engineered heavy chain antibody variable region that is less immunogenic in a human as compared to the immunogenicity of the donor antibody heavy chain variable region, with the proviso that the engineered antibody variable region does not comprise a heavy chain polypeptide variable region comprising the complete amino acid sequence depicted in SEQ ID NO:5 or SEQ ID NO:7.

123-158. (canceled)

159. An engineered antibody comprising: (i) a light chain polypeptide and (ii) a heavy chain polypeptide, wherein the light chain polypeptide comprises the following amino acid sequence segments in order: LFR1-LCDR1-LFR2-LCDR2-LFR3-LCDR3-LFR4,
wherein LFR1 comprises the amino acid sequence depicted in SEQ ID NO:9, but with zero to three amino acid substitutions; LFR2 comprises the amino acid sequence depicted in SEQ ID NO:10 or SEQ ID NO:18, but with zero to three amino acid substitutions; LFR3 comprises the amino acid sequence depicted in SEQ ID NO:11, but with zero to three amino acid substitutions; and LFR4 comprises the amino acid sequence depicted in SEQ ID NO:12, but with zero to three amino acid substitutions;
wherein LCDR1 comprises the amino acid sequence of light chain CDR1 from a donor antibody, LCDR2 comprises the amino acid sequence of light chain CDR2 from a donor antibody, and LCDR3 comprises the amino acid sequence of light chain CDR3 from a donor antibody;
with the proviso that the light chain polypeptide does not comprise the amino acid sequence depicted in SEQ ID NO:2 or SEQ ID NO:8; and
wherein the heavy chain polypeptide comprises the following amino acid sequence segments in order: HFR1-HCDR1-HFR2-HCDR2-HFR3-HCDR3-HFR4,
wherein HFR1 comprises the amino acid sequence depicted in SEQ ID NOs: 13, 17, or 19, but with zero to three amino acid substitutions; HFR2 comprises the amino acid sequence depicted in SEQ ID NO:14, but with zero to three amino acid substitutions; HFR3 comprises the amino acid sequence depicted in SEQ ID NO:15, but with zero to three amino acid substitutions; and HFR4 comprises the amino acid sequence depicted in SEQ ID NO:16, but with zero to three amino acid substitutions,
wherein HCDR1 comprises the amino acid sequence of heavy chain CDR1 from a donor antibody, HCDR2 comprises the amino acid sequence of heavy chain CDR2 from a donor antibody, and HCDR3 comprises the amino acid sequence of heavy chain CDR3 from a donor antibody,
with the proviso that the heavy chain polypeptide does not comprise the amino acid sequence depicted in SEQ ID NO:5 or SEQ ID NO:7; and
wherein the engineered antibody is less immunogenic in a human as compared to the donor antibody or antibodies and the engineered antibody binds to the same antigen as the donor antibody or antibodies.

160-161. (canceled)


SEQUENCE LISTING (45 sequences)

SEQ ID NO: 1 - protein, 214 aa, Artificial Sequence [sequence omitted]

SEQ ID NO: 2 - protein, 107 aa, Artificial Sequence [sequence omitted]

SEQ ID NO: 3 - protein, 107 aa, Artificial Sequence [sequence omitted]

SEQ ID NO: 4 - protein, 448 aa, Artificial Sequence
  Description of Artificial Sequence Synthetic amino acid sequence for the eculizumab heavy chain polypeptide
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 122 aa, Artificial Sequence [sequence omitted]

SEQ ID NO: 6 - protein, 326 aa, Artificial Sequence
  Description of Artificial Sequence Synthetic amino acid sequence for the eculizumab heavy chain polypeptide constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 126 aa, Artificial Sequence
  Description of Artificial Sequence Synthetic amino acid sequence of the H20C3 heavy chain variable region
QVQLVQSGAEVKKPGASVKVSCKASGYTFTSYYIHWVRQAPGQGLEWMGIINPSGGSTNYAQKFQGRVTMTRDTSTSTVYMELSSLRSEDTAVYYCARAPHQRTRIAARPGEGDSWGQGTLVTVSS

SEQ ID NO: 8 - protein, 107 aa, Artificial Sequence
  Description of Artificial Sequence Synthetic amino acid sequence for the I.23 light chain variable region
DIQMTQSPSSLSASVGDRVTITCRASQSISNYLNWYQRKPGKAPKLLIYAASSLQSGVPSRFSGSGSGTDFTLTISSLQPEDFATYYCQQSYNTPWTFGQGTKVEIK

SEQ ID NO: 9 - protein, 23 aa, Artificial Sequence
  Description of Artificial Sequence Synthetic amino acid sequence for the eculizumab light chain framework region 1 (Kabat)
DIQMTQSPSSLSASVGDRVTITC

SEQ ID NO: 10 - protein, 15 aa, Artificial Sequence
  Description of Artificial Sequence Synthetic amino acid sequence for the eculizumab light chain framework region 2 (Kabat)
WYQQKPGKAPKLLIY

SEQ ID NO: 11 - protein, 32 aa, Artificial Sequence
  Description of Artificial Sequence Synthetic amino acid sequence for the eculizumab light chain framework region 3 (Kabat)
GVPSRFSGSGSGTDFTLTISSLQPEDFATYYC

SEQ ID NO: 12 - protein, 10 aa, Artificial Sequence
  Description of Artificial Sequence Synthetic amino acid sequence for the eculizumab light chain framework region 4 (Kabat)
FGQGTKVEIK

SEQ ID NO: 13 - protein, 30 aa, Artificial Sequence
  Description of Artificial Sequence Synthetic amino acid sequence for the eculizumab heavy chain framework region 1 (Kabat)
QVQLVQSGAEVKKPGASVKVSCKASGYIFS

SEQ ID NO: 14 - protein, 14 aa, Artificial Sequence
  Description of Artificial Sequence Synthetic amino acid sequence for the eculizumab heavy chain framework region 2 (Kabat)
WVRQAPGQGLEWMG

SEQ ID NO: 15 - protein, 32 aa, Artificial Sequence
  Description of Artificial Sequence Synthetic amino acid sequence for the eculizumab heavy chain framework region 3 (Kabat)
RVTMTRDTSTSTVYMELSSLRSEDTAVYYCAR

SEQ ID NO: 16 - protein, 11 aa, Artificial Sequence
  Description of Artificial Sequence Synthetic amino acid sequence for the eculizumab heavy chain framework region 4 (Kabat)
WGQGTLVTVSS

SEQ ID NO: 17 - protein, 25 aa, Artificial Sequence [sequence omitted]

SEQ ID NO: 18 - protein, 15 aa, Artificial Sequence
  Description of Artificial Sequence Synthetic amino acid sequence for the I.23 light chain framework region 2 (Kabat)
WYQRKPGKAPKLLIY

SEQ ID NO: 19 - protein, 30 aa, Artificial Sequence
  Description of Artificial Sequence Synthetic amino acid sequence for the H20C3 heavy chain framework region 1 (Kabat)
QVQLVQSGAEVKKPGASVKVSCKASGYTFT

SEQ ID NO: 20 - protein, 25 aa, Artificial Sequence
  Description of Artificial Sequence Synthetic amino acid sequence for the eculizumab light chain framework region 1 (Chothia)
DIQMTQSPSSLSASVGDRVTITCGA

SEQ ID NO: 21 - protein, 17 aa, Artificial Sequence
  Description of Artificial Sequence Synthetic amino acid sequence for the eculizumab light chain framework region 2 (Chothia)
LNWYQQKPGKAPKLLIY

SEQ ID NO: 22 - protein, 38 aa, Artificial Sequence
  Description of Artificial Sequence Synthetic amino acid sequence for the eculizumab light chain framework region 3 (Chothia)
NLADGVPSRFSGSGSGTDFTLTISSLQPEDFATYYCQN

SEQ ID NO: 23 - protein, 11 aa, Artificial Sequence
  Description of Artificial Sequence Synthetic amino acid sequence for the eculizumab light chain framework region 4 (Chothia) and the amino acid sequence for the I.23 light chain framework region 4 (Chothia)
TFGQGTKVEIK

SEQ ID NO: 24 - protein, 25 aa, Artificial Sequence
  Description of Artificial Sequence Synthetic amino acid sequence for the I.23 light chain framework region 1 (Chothia)
DIQMTQSPSSLSASVGDRVTITCRA

SEQ ID NO: 25 - protein, 17 aa, Artificial Sequence
  Description of Artificial Sequence Synthetic amino acid sequence for the I.23 light chain framework region 2 (Chothia)
LNWYQRKPGKAPKLLIY

SEQ ID NO: 26 - protein, 38 aa, Artificial Sequence
  Description of Artificial Sequence Synthetic amino acid sequence for the I.23 light chain framework region 3 (Chothia)
SLQSGVPSRFSGSGSGTDFTLTISSLQPEDFATYYCQQ

SEQ ID NO: 27 - protein, 20 aa, Artificial Sequence
  Description of Artificial Sequence Synthetic amino acid sequence for the eculizumab heavy chain framework region 2 (Chothia)
WIQWVRQAPGQGLEWMGEIL

SEQ ID NO: 28 - protein, 43 aa, Artificial Sequence
  Description of Artificial Sequence Synthetic amino acid sequence for the eculizumab heavy chain framework region 3 (Chothia)
STEYTENFKDRVTMTRDTSTSTVYMELSSLRSEDTAVYYCARY

SEQ ID NO: 29 - protein, 12 aa, Artificial Sequence
  Description of Artificial Sequence Synthetic amino acid sequence for the eculizumab heavy chain framework region 4 (Chothia)
VWGQGTLVTVSS

SEQ ID NO: 30 - protein, 20 aa, Artificial Sequence
  Description of Artificial Sequence Synthetic amino acid sequence for the H20C3 heavy chain framework region 2 (Chothia)
YIHWVRQAPGQGLEWMGIIN

SEQ ID NO: 31 - protein, 43 aa, Artificial Sequence
  Description of Artificial Sequence Synthetic amino acid sequence for the H20C3 heavy chain framework region 3 (Chothia)
STNYAQKFQGRVTMTRDTSTSTVYMELSSLRSEDTAVYYCARA

SEQ ID NO: 32 - protein, 12 aa, Artificial Sequence
  Description of Artificial Sequence Synthetic amino acid sequence for the H20C3 heavy chain framework region 4 (Chothia)
SWGQGTLVTVSS

SEQ ID NO: 33 - protein, 112 aa, Artificial Sequence
  Description of Artificial Sequence Synthetic amino acid sequence of a murine light chain variable region of an anti-human C5a antibody
DIVMTQSPASLAVSLGQRATISCRASESVDSYGNSFMHWYQQKPGQPPKLLIYRASNLESGIPARFSGSGSRTDFTLTINPVEADDVATYYCQQSNEDPYTFGGGTKLEIKR

SEQ ID NO: 34 - protein, 15 aa, Artificial Sequence
  Description of Artificial Sequence Synthetic amino acid sequence for the light chain CDR1 of a murine anti-C5a antibody (Combined Kabat-Chothia definition)
RASESVDSYGNSFMH

SEQ ID NO: 35 - protein, 7 aa, Artificial Sequence
  Description of Artificial Sequence Synthetic amino acid sequence for the light chain CDR2 of a murine anti-C5a antibody (Combined Kabat-Chothia definition)
RASNLES

SEQ ID NO: 36 - protein, 9 aa, Artificial Sequence
  Description of Artificial Sequence Synthetic amino acid sequence for the light chain CDR3 of a murine anti-C5a antibody (Combined Kabat-Chothia definition)
QQSNEDPYT

SEQ ID NO: 37 - protein, 121 aa, Artificial Sequence
  Description of Artificial Sequence Synthetic amino acid sequence of a murine heavy chain variable region of an anti-human C5a antibody
EVQLQQSGPELVKPGSSVKISCKASGYTFTDYSMDWVKQSHGKSLEWIGAINPNSGGTNYSQKFKDKATLTVDKSSSTAYMELRSLTSEDSAVYYCASSGSYDGYYAMDYWGQGTSVTVSS

SEQ ID NO: 38 - protein, 10 aa, Artificial Sequence
  Description of Artificial Sequence Synthetic amino acid sequence for the heavy chain CDR1 of a murine anti-C5a antibody (Combined Kabat-Chothia definition)
GYTFTDYSMD

SEQ ID NO: 39 - protein, 17 aa, Artificial Sequence
  Description of Artificial Sequence Synthetic amino acid sequence for the heavy chain CDR2 of a murine anti-C5a antibody (Combined Kabat-Chothia definition)
AINPNSGGTNYSQKFKD

SEQ ID NO: 40 - protein, 12 aa, Artificial Sequence
  Description of Artificial Sequence Synthetic amino acid sequence for the heavy chain CDR3 of a murine anti-C5a antibody (Combined Kabat-Chothia definition)
SGSYDGYYAMDY

SEQ ID NO: 41 - protein, 111 aa, Artificial Sequence
  Description of Artificial Sequence Synthetic amino acid sequence of a light chain variable region of a humanized anti-human C5a antibody
DIQMTQSPSSLSASVGDRVTITCRASESVDSYGNSFMHWYQQKPGKAPKLLIYRASNLESGVPSRFSGSGSGTDFTLTISSLQPEDFATYYCQQSNEDPYTFGGGTKVEIK

SEQ ID NO: 42 - protein, 10 aa, Artificial Sequence
  Description of Artificial Sequence Synthetic amino acid sequence for the light chain framework region 4 of a humanized anti-C5a antibody (Combined Kabat-Chothia definition)
FGGGTKVEIK

SEQ ID NO: 43 - protein, 121 aa, Artificial Sequence
  Description of Artificial Sequence Synthetic amino acid sequence of a heavy chain variable region of a humanized anti-human C5a antibody
QVQLVQSGAEVKKPGASVKVSCKASGYTFTDYSMDWVRQAPGQGLEWMGAINPNSGGTNYNQKFKDRVTMTRDTSTSTVYMELSSLRSEDTAVYYCARSGSYDGYYAMDYWGQGTTVTVSS

SEQ ID NO: 44 - protein, 17 aa, Artificial Sequence
  Description of Artificial Sequence Synthetic amino acid sequence for the heavy chain CDR2 of a humanized anti-C5a antibody (Combined Kabat-Chothia definition)
AINPNSGGTNYNQKFKD

SEQ ID NO: 45 - protein, 11 aa, Artificial Sequence
  Description of Artificial Sequence Synthetic amino acid sequence for the heavy chain framework region 4 of a humanized anti-C5a antibody (Combined Kabat-Chothia definition)
WGQGTTVTVSS